(19) United States
(12) Patent Application Publication
(10) Pub. No.: US 2007/0141066 A1
(43) Pub. Date: Jun. 21, 2007

(54) Method for Diagnosing, Prognosing and Treating Glioma

(75) Inventors: Heidi S. Phillips, Palo Alto, CA (US); William F. Forrest III, San Carlos, CA (US); Samir Kharbanda, San Francisco, CA (US); Thomas D. Wu, San Francisco, CA (US)

Correspondence Address: GENENTECH, INC., 1 DNA WAY, SOUTH SAN FRANCISCO, CA, 94080, US

(73) Assignee: Genentech, Inc., South San Francisco, CA (US)

(21) Appl. No.: 11/609,071

(22) Filed: Dec. 11, 2006

Related U.S. Application Data
(60) Provisional application No. 60/750,944, filed on Dec. 16, 2005.

(51) Int. Cl. C12Q 1/68; G01N 33/574; G06F 19/00; A61K 39/395; A61K 33/24

(52) U.S. Cl. 424/155.1; 435/7.23; 702/19; 435/6; 424/649

(57) ABSTRACT

The invention provides generally a method of monitoring, diagnosing, prognosing and treating glioma. Specifically, the invention provides for three (3) prognostic subclasses of glioma, which are differentially associated with activation of the akt and notch signaling pathways. Tumor displaying neural or proneural PN lineage markers (including notch pathway elements) show longer median patient survival, while the two remaining tumor markers Prolif and Mes are associated with shortened survival. Tumors classified in this manner may also be treated with the appropriate PN- Prolif- or Mes-therapeutic corresponding to the subclassification in combination with anti-mitotic agents, anti-angiogenic agents, Akt antagonists, and neural differentiation agents. Alternatively, the invention also provides for method of prognosing and diagnosing glioma with a two-gene model based on the expression levels of PTEN and DLL3.

### RELATED APPLICATIONS

The present claims priority under 35 U.S.C. § 119(e) to U.S. Ser. No. 60/750,944 filed Dec. 16, 2005.

### FIELD OF THE INVENTION

The present invention is directed to methods for diagnosing, prognosing and treating cancer, specifically, glioma.

### BACKGROUND OF THE INVENTION

Malignant tumors (cancers) are the second leading cause of death in the United States, after heart disease (Boring et al., CA Cancer J. Clin. 43:7 (1993)). Cancer is characterized by the increase in the number of abnormal, or neoplastic, cells derived from a normal tissue which proliferate to form a tumor mass, the invasion of adjacent tissues by these neoplastic tumor cells, and the generation of malignant cells which eventually spread via the blood or lymphatic system to regional lymph nodes and to distant sites via a process called metastasis. In a cancerous state, a cell proliferates under conditions in which normal cells would not grow. Cancer manifests itself in a wide variety of forms, characterized by different degrees of invasiveness and aggressiveness.

Gliomas are the most common type of primary brain tumors and are typically associated with grave prognosis. High-grade astrocytomas, which include glioblastoma (GBM) and anaplastic astrocytoma (AA), are the most common intrinsic brain tumors in adults. While there has been progress in understanding the molecular genetics of high-grade astrocytomas (Kitange et al., Curr. Opin. Oncol. 15: 197-203 (2003), the cell type(s) of origin are still uncertain and the molecular determinants of disease aggressiveness are not well understood. A better understanding of the cellular origin and molecular pathogenesis of these tumors may identify new targets for treatment of these neoplasms that are nearly uniformly fatal.

The grading of tumors is often critical to an accurate diagnosis and prognosis of disease progression, and gliomas is no exception. Decades of experience has lead to a system of diagnosis of gliomas based on histology. Gliomas are histologically defined based on whether they exhibit primarily astrocytic or oligodendroglial morphology, and are graded by cellularity, nuclear atypia, necrosis, mitotic figures, and micro-vascular proliferation—all features associated with biologically aggressive behavior. This system of diagnosis has been developed over decades of clinical experience with gliomas and has now become the cornerstone of neuro-oncology. Kleihues, P. et al., World Health Organization (“WHO”) classification of tumors, Cancer 88: 2887 (2000). The WHO classification scheme of astrocytic gliomas is divided into four (4) grades. Less malignant tumors fall under grades I (pilocytic astrocytoma) and II (astrocytic glioma), whereas the more malignant tumors are defined under grade III (anaplastic astrocytoma) and grade IV (glioblastoma multiforme). Oligodendrogliomas and mixed gliomas (with both oligodendroglial and astrocytic components) occur in low-grade (WHO grade II) and more malignant variants (WHO grade III).

Until recently, astrocytomas and other gliomas were presumed to arise from glial cells residing within the brain parenchyma. However, new evidence in human and animal studies suggests neural stem cells as an alternate cellular origin of gliomas (Caussinus and Gonzalez, Nat. Genet. 37: 1125 (2005); Singh et al., Cancer Res. 63: 5821-5828 (2003); Zhu et al., Cancer Cell 8: 119 (2005). Mouse models demonstrate that astrocytes or neural stem/progenitor cells can give rise to neoplasms that display the histopathological hallmarks of human gliomas (Bachoo et al., Cancer Cell 1: 269-277 (2002); Uhrbom et al., Cancer Res. 62: 5551-5558 (2002). Demonstrations that the adult human forebrain contains an abundant source of neural stem cells (Sanai et al., Nature 427: 740-744 (2004) and that human GBMs contain tumorigenic neural stem-like cells (Galli et al., Cancer Res. 64: 7011-7021 (2004); Ignatova et al., GLIA 39: 193-206 (2002); Singh et al., Nature 432: 396-401 (2004) indicate that neural stem and/or progenitor cells are a plausible origin for human gliomas and have given rise to speculation that more effective therapies will result from approaches aimed at targeting the stem cell-like component of GBM (Berger et al., Lancet Oncol. 5: 511-514 (2004); Fomchenko and Holland, Exp. Cell Res. 306: 323-329 (2005); Ignatova et al., supra; Oliver and Wechsler-Reya, Neuron 42: 885-888 (2004). Importantly, however, the contribution of stem-like cells to disease progression or therapeutic response has not been established, nor is it clear what proportion of tumor cells exhibit stem-like properties.

As patient prognosis and therapeutic decisions are made in reliance on accurate pathological grading, consistency is a critical attribute. While for the most part reproducible, the present histological based system can result in substantial disagreement between neuropathologists with respect to both type and grade. Louis, D N et al., Am J. Pathol. 159: 779-86 (2001); Prayson R A et al., J. Neurol. Sci. 175: 33-9 (2000); Coons et al., Cancer 79:1381-93 (1997). Moreover, the precise method of grading changes over time. Finally, because it is based on morphology [Burger, Brain Pathol. 12:257-9 (2002)], a biological, rather than molecular end state, the approach is limited in its ability to identify new potential compounds.

Presently, histology-based grading of diffuse infiltrative gliomas is the best predictor of patent survival time. However, histology is neither illustrative of the pathology of gliomas nor very helpful to identify new molecular markers and their use for developing new therapeutics. Moreover, evidence is mounting that the presence of unrecognized, clinically relevant subclasses of diffuse gliomas both with respect to molecular marker expression and therapeutic response. Mischel P S et al., Cancer Biol. Ther. 2:242-7 (2003). It has further been noted from a variety of treatment regimens, that clinical responses to histologically identical tumors can be highly varied. Mischel et al., supra.; Cloughesy, T F et al., Cancer 97: 2381-6 (2003). This underscores how histopathologic evaluation is unrevealing of underlying biology. As oncologists move to molecularly targeted therapies, identification of distinct molecularly defined subgroups becomes increasingly important. However, while specific tumor-associated genes have been studied, individual gene/protein assays alone or even in combination with histologic features have to date not been predictive of survival or helpful in guiding therapeutic decisions. Tortosa, A. et al., Cancer 97: 1063-71 (2003); Reavey-Cantwell, J F et al., J. Neurooncol. 55: 195-204 (2001); Bouvier-Labit, C. et al., Neuropathol. Appl. Neurobiol. 24:381-8 (1998); Stark, A M et al., Zentralbl Neurochir. 64: 30-6 (2003); Li, J. et al., Science 275: 1943-7 (1997).

Several studies have investigated molecular correlates of prognosis and clinical sub-classes in AA and GBM (also known as grade III and IV astrocytoma, respectively). Tumor grade is the most well-established and robust predictor of disease outcome (Prados and Levin, Semin Oncol. 27: 1-10 (2000). Loss of heterozygosity of chromosome (chr) 10q is a more frequent occurrence in GBM than AA and has been associated with short GBM survival (Balesaria et al., Br. J. Can. 81: 1371-1377 (1999); Schmidt et al., J. Neuropathol. Exp. Neurol. 61: 321-328 (2002); Smith et al., J. Nat. Can. Inst. 93: 1246-1256 (2001). Older age at diagnosis is a negative prognostic factor for GBM (Curran et al., J. Natl. Cancer Inst. 85; 704-710 (1993), and molecular markers of outcome differ in older and younger patients (Batchelor et al., Clin. Cancer Res. 10: 228-233 (2004); Smith et al., J. Natl. Cancer Inst. 93: 1246-1256 (2001), suggesting the existence of age-associated molecular subclasses. While p53 mutation and EGFR amplification reportedly define mutually-exclusive GBM subgroups [von Deimling et al, Glia 15, 328-338 (1995); Watanabe et al., Brain Pathology 6, 217-223 (1996)], a recent study challenges the validity of this classification scheme [Okada et al., Cancer Research 63, 413-416 (2003)] and the prognostic value of either p53 mutation or alterations of the EGFR locus is not clear (Heimberger et al., Clinical Cancer Research 11: 1462-1466 (2005); Ushio et al., Frontiers in Bioscience 8: e281-288 (2003). It is clear that a better understanding of the biological basis of these tumors is needed in order to more effectively treat this disease.

Microarray analysis has been identified as a tool that can provide unbiased, quantitative and reproducible tumor evaluation because it can simultaneously evaluate the expression thousands of individual genes. This approach as been applied to many different cancers including gliomas. Mischel, P. S. et al., Oncogene 22: 2361-73 (2003); Kim, S. et al., Mol. Cancer Ther. 1:1229-36 (2002); Ljubimova et al., Cancer Res. 61: 5601-10 (2001); Nutt, C L et al., Cancer Res. 63: 1602-7 (2003); Rickman, D. S. et al., Cancer Res. 61: 6885-91 (2001); Sallinen, S. L. et al., Cancer Res. 60: 6617-22 (2000); Shai, R. et al., Oncogene 22: 4918-23 (2003). Unlike histological evaluation, microarray analysis can identify the underlying genetic variation in the tumors, enhancing tumor classification as well as patient prognostication. Microarray analysis of gliomas has resulted in a classification into more homogenous groups. Freije et al., Cancer Res. 64: 6503-6510 (2004). Moreover, it has also been found to be a superior prognosticator of survival than histological grading. Freije et al., supra.

Expression profiling of malignant gliomas has identified molecular subtypes as well as genes associated with tumor grade progression, and patient survival (Godard et al., Cancer Res. 63: 6613-6625 (2003); Rickman et al., Can. Res. 61: 6885-6891 (2001); van den Boom et al., Am. J. Pathol. 163: 1033-1043 (2003). While GBM and AA continue to be defined on the basis of histologic appearance, the finding that expression profiling predicts outcome better than histological features (Freije et al., supra.; Nutt et al., Clin. Can. Res. 11: 2258-2264 (2003) provides support for the hypothesis that neoplasms defined as AA and GBM on a morphologic basis represent a mix of molecular genetic subtypes. Given the possibility that molecularly-distinct disease entities may exhibit different clinical responses to targeted anti-cancer agents, a greater understanding of the behavior of molecularly-defined subsets of tumors may aid in the development of more effective therapeutics.

Malignant gliomas are believed to develop as a result of stepwise accumulations of genetic lesions. For example, anaplastic astrocytoma typically exhibits: (1) loss of a functional p53 pathway, usually by p53 mutation; (2) loss of a functional p6/pRb pathway, typically by deletion of the p16/ARF locus; and (3) ras pathway activation by means other than ras mutation; and (4) telomerase reactivation, which is rarely seen in normal human astrocytes (NHAs) or grade II glioma. Cavenee et al., “Diffuse infiltrating astrocytomas,” in Pathology and Genetics of Tumors of the Nervous System, W. K. Cavnee and P. Kleihues, Eds., pp. 9-51. Lyon:IRAC Press (2000); Ichimura et al., Cancer Res. 60: 417-424 (2000); Feldkamp et al., Neurosurgery 45: 1442-1453 (1999). Glioblastoma multiforme (GBM), in addition to the alteration in the p53 and p16/pRb pathway noted above, also frequently contain PTEN disruptions leading to activation of the Akt pathway. Hass-Kogan et al., Curr. Biol. 8: 1195-1198 (2000), Holland et al., Nat. Genet. 25: 55-57 (2000). Through the effect on downstream targets, Akt can lead to reduced levels of cell cycle inhibitor [Datta et al., Cell 91: 231-241 (1997); Pap et al., J. Biol. Chem. 273: 19929-19932 (1998); Brunet et al., Cell 96: 857-868 (1999); Kops et al., Nature, 398: 630-634 (1999); Medema et al., Nature 404: 782-787 (2000)], as well as increase vascular endothelial growth factor levels under hypoxic conditions. Mazure et al., Blood 90: 3322-3331 (1997). Akt can suppress apoptosis, deregulate cell cycling and alter angioenic potential. Moreover, 80% of all GBM tumors are observed to express elevated levels of Akt. In light of the known effect of Akt on cell physiology and elevated expression in GBM, activation of Akt is strongly implicated in the development of GBM. Hass-Kogan, supra.; Holland, supra.

The tumor suppressor gene Pten encodes a phosphatase that is frequently mutated, deleted or otherwise somatically inactivated in various human cancers, including glioblastoma. Li et al., Science 275: 1943 (1997). In addition to carcinogenesis, Pten may also play important roles in brain development, as suggested by its ubiquitous central nervous system (CNS) expression pattern in embryos. Gimm et al., Hum. Mol Genet. 9: 1633 (2000); Luukko et al., Mech. Dev. 83: 187 (1999), as well as by neurological disorders associated with Pten germ-line mutations in humans. While the early embryonic lethality of conventional Pten^{-/-} knock out mice has impeded studies on the function of Pten in early brain development, [Di Cristofano et al., Nature Genet. 19: 348 (1998) and Stambolic et al., Cell 95: 29 (1998)] promoter driven transgenic knockout animals, such as that resulting from Cre and loxp transgenes in the parental animals has resulted in the suggestion that Pten negatively regulates neural stem cell production. Groszer et al., Science 294: 2186-2189 (2001).

The Notch signaling pathway has been implicated in carcinogenesis of many cancers, including Hodgkin's disease, T-cell lymphoma, and breast, cervical, pancreatic and colon cancer. 40-44 Jundt, F. et al., Blood 99:3398-403 (2002); Pear, W. S. et al., J. Exp. Med. 183: 2283-91 (1996); Weijzen S., et al., Nat. Med. 8: 979-86 (2002); Weijzen et al., J. Cell Physiol. 194: 356-62 (2003); Miyamoto, Y., et al., Cancer Cell 3: 565-76 (2003); Nickoloff, B. J. et al., Oncogene 22: 6598-608 (2003). The notch family of receptors consists of heterodimeric transmembrane proteins intimately involved in the determination of cell fate. Depending on the cell type, notch signaling can positively or negatively influence proliferation, differentiation and apoptosis Artavanis-Tsakonas, S. et al., Science 284: 770-6 (1992); Miele, L. et al., J. Cell Physiol. 181: 393-409 (1999). To date, four notch receptors have been identified in humans (i.e., notch 1-4) with five corresponding ligands, including delta-like 1 (d11-1), delta-like-3 (d11-3), delta-like-4 (d11-4), jagged-1 and jagged-2. The notch pathway interacts and overlaps with other critical cancer pathways such as hedgehog (Hallahan et al., Cancer Res. 64: 7794-7800 (2004) and Ras. Fitzgerald, K. et al., Oncogene 19: 4191-8 (2000); Ruiz-Hidalgo, R. J. et al., J. Oncol. 14: 777-83 (1999). However, the role of notch in cancers appears to be complex, based on factors such as tissue type. While notch-1 activity is necessary to maintain a cancerous phenotype in ras-transformed human cells (Weijzen, S. et al., Nat. Med. 8: 979-86 (2002), notch-1 signaling was found to have a tumor-supressive effect on murine skin tumors and in non-small cell lung cancer. Wolfer et al., Nat. Genet. 33: 416-21 (2003); Sriuranpong, V. et al., Cancer Res. 61: 3200-5 (2001). The findings suggest a variable role for notch signaling in cancer.

Notch signaling both inhibits differentiation and promotes proliferation in the developing cerebellum. Solecki, D J. et al., Neuron 31: 557-68 (2001). Moreover, notch signaling has been specifically associated with gliomas. Specifically, the notch ligands delta-like-1 and jagged-1 and notch-1 receptor expression is enhanced in both glioma cell lines and human glioma tumors, and their down regulation induced apoptosis and inhibited proliferation in multiple glioma cell lines and prolonged survival in animal models. Purow et al., Cancer Res. 65(6): 2353-2363 (2005).

However, the role of notch signaling in tumorigenesis can vary. For example, it has been observed that Notch-1 activity inhibits the proliferation of medulloblastoma cells, whereas Notch-2 activity promotes their growth. Fan et al., Cancer Res. 64: 7787-7793 (2004). Further complicating our understanding of the notch signaling pathway, it has been recently suggested that the notch ligand D113 is associated with notch inactivation. Ladi et al., J. Cell Biol. 170: 983-992 (2005). Thus, at the present time, the understanding of the role of notch in tumorigenesis is at best incomplete.

While gene expression profiling, such as that provided by microarray analaysis, can identify a panel of gene expression in gliomas that is predictive of survival, no analysis to date has yet identified individual gene expression to be effective prognosticators of survival. For example, in a microarray analysis of stage III and IV glioma tumor tissue removed from 74 patients, identified 595 differentially expressed genes associated with survival. Freije et al., Cancer Res. 64: 6503-6510 (2004). From this group, 44 of the most strongly and consistently differentially expressed genes were identified. This group was further narrowed to 16 individual genes, which were further evaluated by reverse transcription-PCR. Additional modeling identified enhanced expression of the notch ligand DLL3 as one of 6 genes associated with enhanced survival. However, while this study demonstrated the prognostic and diagnostic value of genetic profiling resulting from microarray analysis, it did not result in the identification of any individual genes as having prognostic value.

Applicants identify herein three (3) novel prognostic subclasses of glioma and show them to be differentially associated with activation of the akt and Notch signaling pathways (Prolif, Mes). One tumor class, displaying neural or proneural (PN) lineage markers and Notch pathway elements, shows longer median survival. In contrast, the two (2) remaining tumor classes, characterized by markers of proliferation or mesenchyme, are associated with shorter survival.

Applicants further identity herein a two-gene model, and reveal that higher expression of both PTEN and DLL3 correlates with longer survival, demonstrating the impact of Akt and Notch signaling pathways on tumor aggressiveness. Moreover, upon recurrence, some tumors that originally presented with proneural or proliferative phenotypes shift to the mesenchymal class, thereby suggesting that these molecularly-defined groups may represent alternate differentiation states or stages of tumor progression. This two-gene model is distinct from the predictive value disclosed in Freije et al. because the prognostic value of DLL3 plus PTEN in a two gene model of survival was shown to be statistically-significant in two independent datasets. BMP2 is a marker identified by Freije et al. as a marker of the same subclass of tumor as DLL3. When BMP2 is substituted for DLL3 in the two gene model, it fails to reproduce the findings seen with DLL3.

Applicants have further discovered that the activation status of the Notch or Akt pathway is a major determent of tumor aggressivness and may predict response to targeted therapies.

Finally, Applicants have identified that poor prognosis tumors are characterized by neural stem cell markers and Akt pathway signaling and angiogenesis or proliferation. Notch pathway signaling, and markers of committed neuronal precursors characterize better prognosis tumors. Normal brain has little proliferation, angiogenesis, or Notch and Akt signaling, but is characterized by high expression of neuronal markers. Good prognosis PN tumors may show patches of cells with poor prognosis Mes markers and PN tumors may recur with Mes phenotype, thus suggesting that poor prognosis tumors may be converted into better prognosis PN-like tumors by blocking the appropriate biological processes such as Akt signaling, angiogenesis or proliferation. These findings suggest that blocking Akt signaling, angiogenesis or proliferation in combination with blocking of Notch signaling or other treatments that induce neuronal differentiation might slow the growth of glioma tumors.

### SUMMARY

The present invention provides generally for a method of monitoring, diagnosing, prognosing and treating glioma. In one embodiment, the invention provides for three (3) prognostic subclasses of glioma, which are differentially associated with activation of the akt and Notch signaling pathways. The tumor class displaying neural or proneural (PN) lineage markers and Notch pathway elements, shows longer median patient survival. In contrast, the markers of proliferation (Prolif) or mesenchyme (Mes), are associated with shorter survival. In another embodiment, the invention provides for a two gene model of glioma, wherein relatively high expression levels of both PTEN and DLL3 are indicative of lengthened survival and a low expression of PTEN (regardless of DLL3) is indicative of shortened survival.

In another embodiment, the present invention provides for a method of treating glioma comprising: (i) measuring the expression of a set of glioma determinative markers (“GDM”) in a tumor sample, (ii) determining the subclassification proneural (PN), proliferative (Prolif) or mesenchymal (Mes) expression signature of the set, wherein: (I) tumors exhibiting a Prolif subclassification are treated with a combined therapy comprising contacting with effective amounts of (a) an Akt antagonist and/or a Prolif-antagonist and/or anti-mitotic agent, and (b) a neural differentiation agent; (II) tumors exhibiting a Mes subclassification are treated with a combined therapy comprising contacting with effective amounts of (a) an Akt and/or Mes-antangonist and/or anti-angiogenic agent, and (b) a neural differentiation agent; and (III) tumors exhibiting a PN subclassification are treated with a combined therapy comprising contacting with effective amounts of: (1) a PN-antagonist and/or (2) a neural differentiation agent optionally in combination with one or more of the following: (3) an Akt antagonist, (4) anti-mitotic agent, and (5) Mes antagonist and/or anti-angiogenic agent. In a specific aspect, the Akt antagonist is selected from the group consisting of: antagonists of akt1, akt2, akt3, antagonists of regulatory or catalytic domain of PIK3, PD1, FRAP, RPS6KB1, SGK, EGFR, IGFR, and activators, stimulators or restoratives of PTEN, INPP5D or INPPL1. In another specific aspect, the Prolif-antagonist is selected from the group consisting of: antagonists of any of the Prolif markers indicated in Table A. In a further specific aspect, the anti-mitotic agent is selected from the group consisting of: temozolamide, BCNU, CCNU, lomustine, gliadel, etoposide, carmustine, ironotecan, topotecan, procarbazine, cisplatin, carboplatin, cyclophosphamide, vincristine, doxorubicin, dactinomycin, bleomycin, plicamycin, methotrexate, cytarabine, paclitaxel, auristatins, maytansinoids. In yet a further specific aspect, the Mes-antagonist is selected from the group consisting of: antagonists of any of the Mes markers indicated in Table A. In yet a further specific aspect, the anti-angiogenic agent is selected from the group consisting of: VEGF antagonists, anti-VEGF antibody, VEGFR1 and VEGFR2 antagonists. In yet a further aspect, the PN-antagonist is selected from the group consisting of: antagonist of any of the PN markers indicated in Table A, with the exception of DLL3, Nog, Olig1, Olig2, THR and ASCL1. In yet a further specific aspect, the neural differentiation agent is selected from the group consisting of: MAP2, beta-tubulin, GAD65 and GAP43. Example neural differentiation agents include, but are not limited to: retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-4, jagged 1 antagonist, jagged 2 antagonist; numb agonist or numb-like agonist.

In another embodiment, the present invention provides for a method of treating glioma comprising contacting with effective amounts of a (1) a neural differentiation agent in combination with one or more of the following: (3) an Akt antagonist, (4) anti-mitotic agent, and (5) Mes antagonist and/or anti-angiogenic agent. In a specific aspect, the Akt antagonist is selected from the group consisting of: antagonists of akt1, akt2, akt3, antagonists of regulatory or catalytic domain of PIK3, PD1, FRAP, RPS6KB1, SGK, EGFR, IGFR, and activators, stimulators or restoratives of PTEN, INPP5D or INPPL1. In another specific aspect, the Prolif-antagonist is selected from the group consisting of: antagonists of any of the Prolif markers indicated in Table A. In a further specific aspect, the anti-mitotic agent is selected from the group consisting of: temozolamide, BCNU, CCNU, lomustine, gliadel, etoposide, carmustine, ironotecan, topotecan, procarbazine, cisplatin, carboplatin, cyclophosphamide, vincristine, doxorubicin, dactinomycin, bleomycin, plicamycin, methotrexate, cytarabine, paclitaxel, auristatins, maytansinoids. In yet a further specific aspect, the Mes-antagonist is selected from the group consisting of: antagonists of any of the Mes markers indicated in Table A. In yet a further specific aspect, the anti-angiogenic agent is selected from the group consisting of: VEGF antagonists, anti-VEGF antibody, VEGFR1 and VEGFR2 antagonists. In yet a further aspect, the PN-antagonist is selected from the group consisting of: antagonist of any of the PN markers indicated in Table A, with the exception of DLL3, Nog, Olig1, Olig2, THR and ASCL1. In yet a further specific aspect, the neural differentiation agent is selected from the group consisting of: MAP2, beta-tubulin, GAD65 and GAP43. Example neural differentiation agents include, but are not limited to: retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-4, jagged 1 antagonist, jagged 2 antagonist; numb agonist or numb-like agonist.

In another embodiment, the present invention provides a method of prognosing and/or diagnosing glioma comprising (i) measuring the expression of a set of glioma determinative markers (“GDM”) in a tumor sample, (ii) determining the subclassification, proneural (PN), proliferative (Prolif) or mesenchymal (Mes) expression signature in the set, followed by (iii) prognosing or diagnosing disease outcome, wherein a subclassification of Prolif or Mes is indicative of a poorer prognosis or statistically elevated chance of survival time less than the median of the reference sample population and a subclassification of PN is indicative of a better prognosis or statistically elevated chance of survival time greater than the median of the reference sample population. In a specific aspect, the subclassification is carried out using hierarchical clustering. In another specific aspect, the subclassification is carried out using k means clustering. In yet another specific aspect, the subclassification is carried out by a voting scheme. In yet a further specific aspect, the subclassification is carried out by a comparison of GDMs in the tumor to GDMs in a reference set of tumors.

In yet a further embodiment, the invention provides for a method of monitoring or diagnosing glioma comprising comparing the expression signature of a set of glioma determinative markers (“GDM”) in at least two tumor samples from a patient:
(i) measuring the expression of GDM in a first tumor sample at a first point in time;
(ii) measuring the expression of GDM in a second tumor sample at a second later point in time;
(iii) determining the morphological subclassification as proneural (PN), proliferative (Prolif) or mesenchymal (Mes) expression signature of the GDM in the tumor samples;
wherein a transition from the PN to Prolif to Mes subclassification from the first to the second tumor sample is indicative of increased severity or progression of said tumor.

In another embodiment, the present invention provides for a method of inhibiting the growth of a glioma tumors comprising: (i) measuring the expression of a set of glioma determinative markers (“GDM”) in a tumor sample, (ii) determining the subclassification proneural (PN), proliferative (Prolif) or mesenchymal (Mes) expression signature of the set, wherein: (I) tumors exhibiting a Prolif subclassification are treated with a combined therapy comprising contacting with effective amounts of (a) an Akt antagonist and/or a Prolif-antagonist and/or anti-mitotic agent, and (b) a neural differentiation agent; (II) tumors exhibiting a Mes subclassification are treated with a combined therapy comprising contacting with effective amounts of (a) an Akt and/or Mes-antagonist and/or anti-angiogenic agent, and (b) a neural differentiation agent; and (III) tumors exhibiting a PN subclassification are treated with a combined therapy comprising contacting with effective amounts of: (1) a PN-antagonist and/or (2) a neural differentiation agent optionally in combination with one or more of the following: (3) an Akt antagonist, (4) an anti-mitotic agent, and (5) a Mes antagonist and/or anti-angiogenic agent; and wherein the result is the reduced size or growth of the tumor. In a specific aspect, the Akt antagonist is selected from the group consisting of: antagonists of akt1, akt2, akt3, antagonists of regulatory or catalytic domain of PIK3, PD1, FRAP, RPS6KB1, SGK, EGFR, IGFR, and activators, stimulators or restoratives of PTEN, INPP5D or INPPL1. In another specific aspect, the Prolif-antagonist is selected from the group consisting of: antagonists of any of the Prolif markers indicated in Table A. In a further specific aspect, the anti-mitotic agent is selected from the group consisting of: temozolamide, BCNU, CCNU, lomustine, gliadel, etoposide, carmustine, ironotecan, topotecan, procarbazine, cisplatin, carboplatin, cyclophosphamide, vincristine, doxorubicin, dactinomycin, bleomycin, plicamycin, methotrexate, cytarabine, paclitaxel, auristatins, maytansinoids. In yet a further specific aspect, the Mes-antagonist is selected from the group consisting of: antagonists of any of the Mes markers indicated in Table A. In yet a further specific aspect, the anti-angiogenic agent is selected from the group consisting of: VEGF antagonists, anti-VEGF antibody, VEGFR1 and VEGFR2 antagonists. In yet a further aspect, the PN-antagonist is selected from the group consisting of: antagonist of any of the PN markers indicated in Table A, with the exception of DLL3, Nog, Olig1, Olig2, THR and ASCL1. In yet a further specific aspect, the neural differentiation agent is selected from the group consisting of: MAP2, beta-tubulin, GAD65 and GAP43. Example neural differentiation agents include, but are not limited to: retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-4, jagged 1 antagonist, jagged 2 antagonist; numb agonist or numb-like agonist. In a specific aspect, the result of such contact is the decreased proliferation or death of the tumor cell. In another aspect, the antagonist is an antibody or antigen-binding antibody fragment. In yet another specific aspect, the antagonist antibody is a monoclonal antibody, chimeric antibody, humanized antibody or single-chain antibody. In yet a further specific aspect, the antagonist antibody or antigen-binding antibody fragment is conjugated to a growth inhibitory agent or cytotoxic agent such as a toxin, including, for example, a maytansinoid or calicheamicin, an auristatin, an antibiotic, a radioactive isotope, a nucleolytic enzyme, or the like.

In another embodiment, the present invention provides for a method of therapeutically a mammal having a glioma tumor, wherein the method comprises: (i) measuring the expression of a set of glioma determinative markers (“GDM”) in a tumor sample, (ii) determining the subclassification proneural (PN), proliferative (Prolif) or mesenchymal (Mes) expression signature of the set, wherein: (I) tumors exhibiting a Prolif subclassification are treated with a combined therapy comprising administering to the mammal therapeutically effective amounts of (a) an Akt antagonist and/or a Prolif-antagonist and/or anti-mitotic agent, and (b) a neural differentiation agent; (II) tumors exhibiting a Mes subclassification are treated with a combined therapy comprising contacting with effective amounts of (a) an Akt and Mes-antagonist and/or anti-angiogenic agent, and (b) a neural differentiation agent; and (III) tumors exhibiting a PN subclassification are treated with a combined therapy comprising contacting with effective amounts of: (1) a PN-antagonist and/or (2) a neural differentiation agent optionally in combination with one or more of the following: (3) an Akt antagonist, (4) an anti-mitotic agent, and (5) a Mes antagonist and/or anti-angiogenic agent; and wherein the result is the therapeutic treatment of the tumor. In a specific aspect, the antagonist is an antibody, an antigen binding antibody fragment, an oligopeptide, a small molecule antagonist, or an antisense oligonucleotide. In another specific aspect, the antibody is a monoclonal antibody, an antigen-binding antibody fragment, a chimeric antibody, a humanized antibody, or a single-chain antibody. In yet another aspect, the antagonists or agents suitable for use with the present methods, may optionally be conjugated to a growth inhibitory agent or cytotoxic agent such as a toxin, including, for example, a maytansinoid or calicheamicin, an antibiotic, a radioactive isotope, a nucleolytic enzyme, or the like.

In yet a further embodiment, the present invention is directed to a method of determining the expression level of a PN-, Prolif- or Mes- GDM in a sample, wherein the method comprises exposing the sample to PN-, Prolif- or Mes- binding agents and determining the amount of binding of each respective binding agent in the sample, wherein such binding amount is indicative of the expression level of the respective PN-, Prolif- or Mes- GDM in the sample. In a specific aspect, the PN-, Prolif- or Mes-binding agent is (1) an anti-PN- anti-Prolif- or anti-Mes-antibody, (2) a PN-, Prolif- or Mes-binding antibody fragment, (3) a PN-, Prolif- or Mes- binding oligopeptide, (4) a PN-, Prolif- or Mes- small molecule antagonist, or (5) a PN-, Prolif- or Mes- antisense oligonucleotide. In another specific aspect, the preceding antibodies are: (a) a monoclonal antibody, (b) an antigen-binding antibody fragment, (c) a chimeric antibody, (d) a humanized antibody, or (e) a single-chain antibody. In yet another specific aspect, such antibodies are detectably labeled with a molecule of compound that is useful for qualitatively and/or quantitatively determining the location and/or amount of binding of the PN-, Prolif- or Mes- GDM.

In yet a further embodiment of the present invention is directed to a method of prognosing the survival probability in a mammal having a glioma tumor, wherein the method comprises (a) removing a test sample of the tumor, (b) measuring the level of PTEN and DLL3 gene product expression in the test sample and in a set of not less than thirty (30) high grade gliomas for which patient survival times are known, wherein a higher level of expression of both PTEN and DLL3 the test sample is indicative of a statistically elevated chance of survival time greater than the median of the reference sample population and a lower level of expression of either PTEN or DLL3 in the test sample is indicative of a statistically elevated chance of survival time less than the media of the reference sample population.

In yet a further embodiment of the present invention is directed to a method of diagnosing the severity of a glioma tumor in a mammal, wherein the method comprises: (a) contacting a test sample comprising glioma tumor cells or extracts of DNA, RNA, protein or other gene products obtained from the mammal with (i) a first reagent that is an antibody, antigen binding antibody fragment, oligopeptide or small organic molecule that binds to a PTEN GDM and (ii) a second reagent which is an antibody, antigen binding antibody fragment, oligopeptide or small organic molecule that binds to a DLL3 GDM; (b) measuring the amount of complex formation between the first and second reagents with the PTEN GDM and DLL3 GDM in the test sample, respectively, wherein the formation of a high level of PTEN GDM complex formation and high level of DLL3 GDM complex formation is indicative of a mild tumor and the formation of a low level of either PTEN GDM complex or DLL3 GDM is indicative of a severe tumor. In a specific aspect, the first and/or second reagent are/is detectably labeled, attached to a solid support, or the like. In another specific aspect, first reagent is an anti-PTEN antibody, PTEN-binding antibody fragment, or PTEN binding oligopeptide, small molecule, antisense oligonucleotide. In yet another specific aspect, the second reagent may be an anti-DLL3 antibody, DLL3 binding antibody fragment, or DLL3 binding oligopeptide, small molecule or antisense nucleotide. In yet a further specific aspect, the anti-PTEN antibody or anti-DLL3 antibody may be a monoclonal antibody, antigen-binding antibody fragment, chimeric antibody, humanized antibody or single-chain antibody. In yet a further specific aspect, such antibodies are labeled with a molecule or compound that is useful for qualitatively and/or quantitatively determining the location and/or amount of binding of the PTEN or DLL3 binding agent to the cell.

In yet a further embodiment of the invention is directed to the use of: (a) PTEN, or DLL3 polypeptide, or (b) a nucleic acid encoding (a), in the preparation of a medicament useful for the diagnostic detection of a glioma tumor. In a specific aspect, the medicament may be a PTEN binding agent or a DLL3 binding agent. In another specific aspect, the PTEN binding agent may be an anti-PTEN antibody, PTEN-binding antibody fragment, or PTEN binding oligopeptide, small molecule antagonist, antisense oligonucleotide, whereas the DLL3 binding agent may be an anti-DLL3 antibody, DLL3-binding antibody fragment, or DLL3 binding oligopeptide, small molecule, antisense oligonucleotide. In yet another specific aspect, the antibody may be a monoclonal antibody, chimeric antibody, humanized antibody or single-chain antibody. In yet a further specific aspect, such PTEN or DLL3 binding agents are labeled with a molecule or compound that is useful for qualitatively and/or quantitatively determining the location and/or amount of binding of the PTEN or DLL3 binding agent to the cell.

In yet a further embodiment of the invention is directed to the use of: (a) a PN-, Prolif- or Mes- GDM, or (b) a nucleic acid encoding (a), in the preparation of a medicament useful for the diagnostic detection of a glioma tumor. In a specific aspect, the medicament is a PN-, Prolif- or Mes-binding agent. In another specific aspect, the PN-, Prolif- or Mes-binding agent may be: (1) an anti-PN, anti-Prolif or anti-Mes antibody, (2) a PN-, Prolif- or Mes-binding antibody fragment, (3) a PN-, Prolif- or Mes-binding binding oligopeptide, (4) a PN-, Prolif- or Mes-binding small molecule, (5) a PN-, Prolif- or Mes-antisense oligonucleotide. In yet another specific aspect, the anti-PN-, anti-Prolif- or anti-Mes-antibody may be a monoclonal antibody, chimeric antibody, humanized antibody or single-chain antibody. In yet a further specific aspect, such PN-, Prolif- or Mes-binding agents are labeled with a molecule or compound that is useful for qualitatively and/or quantitatively determining the location and/or amount of binding of the PN-, Prolif- or Mes- binding agent to the cell.

### BRIEF DESCRIPTION OF THE DRAWINGS

FIG. 1. Expression profiling reveals three major patterns of gene expression related to survival in high-grade glioma A. Unsupervised clustering of 76 MDA primary grade III & IV astrocytomas by expression of 108 genes positively or negatively correlated with survival (gene clusters labeled positive or negative) reveals three sample clusters (dark grey lines). B. PN, Prolif and Mes tumor subsets (as indicated) of the same 76 samples are identified using 35 signature genes. Centroids from k-means clustering are depicted using z score-normalized gene expression values (scale from −1 to +1). C-E. Kaplan-Meier survival plots of data from MDA, UCSF, and UCLA sample populations. p values from log-rank tests depicted. Light Gray, black and grey lines correspond to PN, Prolif, and Mes subclasses, respectively. Vertical ticks indicate censored survival observations. F. & G. Strong expression of PN and Mes markers is mutually exclusive. Each bar depicts mRNA determinations by microarray (F) or Taqman real-time PCR (G), of four marker genes in an individual sample. Genes include BCAN, DLL3, CHI311/YKL40 (YKL40), and CD44 as indicated. Values displayed represent Z scores for gene expression of individual samples relative to the entire sample set. H. In situ hybridization of BCAN and CHI3L1/YKL40 (YKL40) in tissue microarray cores of 5 glioma cases. Arrows indicate focal CHI3L1/YKL40 expression in BCAN positive cores.

FIG. 2. Most high-grade gliomas are characterized by strong similarity to one of three patterns of signature gene expression and subclass shifts upon disease progression are towards the Mes phenotype. A-C. Three-dimensional graphical representation in which the position occupied by each point represents the similarity (Spearman r) between an individual sample and each of three centroids defined by k-means clustering of the reference (MDA) sample set. A. Nearly all grade III tumors of both astrocytic (black points) or oligodendroglial (light grey points) morphology are most similar to the PN centroid while the population of grade IV tumors (grey points) is more evenly divided by similarity to the centroids. B. Different sets of normal cells or tissues resemble each of the three centroids. Samples are as follows: Fetal brain, adult brain (brain), two neural stem cell lines derived from fetal tissue (NSC1, NSC2), Jurkat, hematopoietic stem cells (HSC) smooth muscle (SmoMusc), endothelial cells (endothel), synovium (synov), and bone. Neural stem cell lines treated by exposure to and withdrawal from the growth factor BDNF are designated NSC1* & NSC2*. C. 26 pairs of matched primary and recurrent astrocytomas (grey=grade IV, black=grade III) are represented. Each pair of matched specimens is connected by an arrow which is solid and bold for instances of signature class shift. D. Genes significantly upregulated in cases which shift into Mes subclass upon recurrence. FC=fold change E. IHC of CHI3L1/YKL40 and OLIG2 in matched primary and recurrent tumors from a case that undergoes a shift from PN to Mes phenotype.

FIG. 3. Tumor subclasses are distinguished by expression of markers for proliferation, angiogenesis, and neurogenesis. A-E. Open circles=brain, grey circles or bars=PN, black triangles or bars=Prolif., grey squares or bars=Mes. A. Prolif tumors are enriched for expression of PCNA and TOP2A, p<1×10⁻⁶ for comparisons with all other groups. B. Mes tumors are distinguished by increased expression of PECAM, VEGF, VEGFR1, and VEGFR2, p<0.05 for comparisons with all other groups. C & D. Relative to PN tumors, Prolif and/or Mes tumors show stronger expression of the neural stem and transmit amplifying markers VIM, NES, TLX, CD133, MELK, and DLX2 (D) and weaker expression of the neuroblast and neuronal markers OLIG2, MAP2, DCX, NeuN, ERBB4, and GAD2 (E). Stars indicate significant differences from PN (p<0.05 Bonferroni post-hoc after ANOVA). E. GFAP expression is significantly decreased in Prolif tumors relative to either PN or MES tumors.

FIG. 4. Copy number changes on chromosome 7, 10, and 19 differ in tumor subclasses and these differences are reflected in expression signatures. A. Frequencies of copy number changes of chromosomes 10, 7 and 19q as a function of tumor signature subclass. For chr 10, tumors are reported as either exhibiting no losses, losses confined to 10q that include the PTEN locus, and losses of essentially all loci. For chr 7, cases are scored as no gains, gains of any portion of chr 7, or gain of all loci. For chr 19q, cases are scored as gains or losses if over ½ of the loci show copy number changes. B. Number of probes sets in PN, Prolif, or Mes tumor marker lists (as labeled) compared to all U133 A&B probesets plotted as a function of chromosome location.

FIG. 5. Prolif and Mes tumors display differential activation of Akt and Notch signaling cascades. PN, Prolif, and Mes tumors denoted by light grey circles, black triangles and grey squares, respectively. (A.) PTEN loss and (B.) EGFR amplification are negatively associated with the PN signature, while (C.) gain of the PIK3R3 locus is positively associated with the Prolif signature. A-C For each sample, the x axis displays the CGH log2 ratio denoting gains or losses at the locus sampled while the y axis indicates correlation to either the PN or Prolif centroid, as indicated r values indicate Pearson and Spearman correlation coefficients between CGH ratios and expression signature centroid similarities. D. Normalized PTEN mRNA levels are lower in poor prognosis tumor subtypes compared to the PN tumor subclass. Horizontal lines denote group means. E.-H. PN tumors show strong overexpression of the Notch pathway elements DLL3, DLL1, HEY2, and ASCL1. I. & J. Tumor subclasses differ in staining for p-Akt and nuclear Notch. For each tumor subtype, the fraction of samples rated as 0, 1, or 2 for p-Akt (J) or nuclear Notch (K) immunostaining is depicted. PN, Prolif, and Mes subtypes are indicated by the three bar graphs, respectively.

FIG. 6. Expression of PTEN and DLL3 predicts survival of high-grade astrocytoma in two independent sample sets (A & B). Plots in left and right panels depict estimated survival functions of each sample population (n=76 for A, n=34 for B) modelled for the instance of PTEN expression at the 20^{th} and 80^{th} percentile (%-ile), respectively. Black and grey lines show estimated survival for samples with DLL3 expression at the ₂₀^{th} and 80^{th}%-ile of expression, respectively.

FIG. 7. Expression signatures of glioma cell lines predict EGF/FGF-independent neurosphere growth. A. Examples of neurosphere cultures derived from 5 cell lines and maintained in the presence or absence of EGF+FGF. Examples of lines rated from 0-4 (as indicated) for growth in the absence of EGF+FGF 4. B. Neurosphere growth ratings for 16 cell lines as a function of correlation of expression signature to the Mes or Prolif centroids.

FIG. 8. Summary of tumor subtypes (A) Major features of tumor subtypes and (B) model depicting parallels between tumor subtypes and stages in neurogenesis.

### DETAILED DESCRIPTION OF THE INVENTION

I. Definitions

The term “glioma” refers to a tumor that arises from glial cells or their precursors of the brain or spinal cord. Gliomas are histologically defined based on whether they exhibit primarily astrocytic or oligodendroglial morphology, and are graded by cellularity, nuclear atypia, necrosis, mitotic figures, and microvascular proliferation—all features associated with biologically aggressive behavior. Astrocytomas are of two main types—high-grade and low-grade. High-grade tumors grow rapidly, are well-vascularized, and can easily spread through the brain. Low-grade astrocytomas are usually localized and grow slowly over a long period of time. High-grade tumors are much more aggressive, require very intensive therapy, and are associated with shorter survival lengths of time than low grade tumors. The majority of astrocytic tumors in children are low-grade, whereas the majority in adults are high-grade. These tumors can occur anywhere in the brain and spinal cord. Some of the more common low-grade astrocytomas are: Juvenile Pilocytic Astrocytoma (JPA), Fibrillary Astrocytoma Pleomorphic Xantroastrocytoma (PXA) and Desembryoplastic Neuroepithelial Tumor (DNET). The two most common high-grade astrocytomas are Anaplastic Astrocytoma (AA) and Glioblastoma Multiforme (GBM).

The terms “glioma determinative marker or markers” (“GDM”) as used herein, refer(s) to cell marker(s) that is/are commonly associated with gliomas. This term encompasses both the gene encoding the marker (e.g., DNA, gene amplification) as well as gene products resulting from the transcription thereof (e.g., mRNA, encoded polypeptides). GDM markers can be distinctive of the proneural (PN), proliferative (Prolif) or mesenchymal (Mes) subclassifications, as shown in Table A.

TABLE A
Glioma Determinative Markers (GDM)

| PN | | | Prolif | | | Mes | | |
|---|---|---|---|---|---|---|---|---|
| | Accession | | | Accession | | | Accession | |
| gene | number | Affy Probe ID | Gene | number | Affy Probe ID | gene | number | Affy Probe ID |
| NCAM1 | AI569787 | 229799_s_at | USP1 | AW499935 | 202412_s_at | MYH9 | AI827941 | 211926_s_at |
| Rpl13 | AI369389 | 229590_at | CDK2 | M68520 | 204252_at | DLC1 | AF026219 | 210762_s_at |
| AP1G2 | BF513474 | 226918_at | SMC4L1 | AL136877 | 201664_at | TIMP1 | NM_003254 | 201666_at |
| JDP1 | NM_021800 | 218976_at | RRM1 | NM_001033 | 201477_s_at | OSMR | NM_003999 | 205729_at |
| C11orf2 | NM_013265 | 217969_at | DHFR | NM_000791 | 202534_x_at | FLJ10945 | NM_018280 | 220681_at |
| FAM13C1 | AI829726 | 226623_at | PMSCL1 | NM_005033 | 205061_s_at | PTRF | BC004295 | 208789_at |
| NCAM1 | M22094 | 217359_s_at | DEK | NM_003472 | 200934_at | HRH1 | D28481 | 205580_s_at |
| ABHD6 | BC001698 | 221552_at | DKFZp761C121 | AL136560 | 223542_at | CHI3L1 | M80927 | 209396_s_at |
| AP2B1 | NM_001282 | 200612_s_at | BRCA1 | NM_007295 | 204531_s_at | PVRL2 | BE867789 | 232079_s_at |
| CYFIP2 | AL161999 | 215785_s_at | HMGB2 | BC000903 | 208808_s_at | C20orf163 | AW298115 | 230283_at |
| VGCNL1 | N49852 | 228608_at | NY-REN-41 | AI458313 | 226287_at | COL4A2 | AA909035 | 211966_at |
| OMG | NM_002544 | 207093_s_at | MCM6 | NM_005915 | 201930_at | T1A-2 | AU154455 | 221898_at |
| ASB13 | NM_024701 | 218862_at | FKSG14 | BC005400 | 222848_at | EMP3 | NM_001425 | 203729_at |
| FLRT1 | AF169675 | 210414_at | KIAA0186 | NM_021067 | 206102_at | TRIM56 | AL512757 | 231876_at |
| CRYAB | AF007162 | 209283_at | RBBP8 | NM_002894 | 203344_s_at | KIAA0963 | NM_014963 | 204166_at |
| SOX8 | BF527050 | 226913_s_at | DHFR | BC000192 | 202532_s_at | DLC1 | AA524250 | 224822_at |
| ATP6V1G2 | BF340635 | 214762_at | TYMS | NM_001071 | 202589_at | S100A11 | NM_005620 | 200660_at |
| GALNT13 | BF346193 | 243779_at | LSM5 | BC005938 | 211747_s_at | FGFRL1 | AF312678 | 223321_s_at |
| HDAC5 | NM_005474 | 202455_at | DHFR | AI144299 | 48808_at | CHI3L1 | M80927 | 209395_at |
| TNKS2 | NM_025235 | 218228_s_at | C20ORF172 | NM_024918 | 219512_at | MAP2K3 | AA780381 | 215498_s_at |
| FGF12 | AL119322 | 214589_at | PIR51 | BE966146 | 204146_at | MSCP | BE677761 | 221920_s_at |
| SKIP | BE549786 | 228509_at | WDHD1 | AK001538 | 216228_s_at | ITGA5 | NM_002205 | 201389_at |
| GALNT13 | R38990 | 236536_at | DONSON | AF232674 | 221677_s_at | CLN2 | AA602532 | 214196_s_at |
| MGC1136 | NM_024025 | 219144_at | ITGB3BP | NM_014288 | 205176_s_at | TMEPAI | NM_020182 | 217875_s_at |
| FGF12 | NM_004113 | 207501_s_at | PMSCL1 | AI346350 | 213226_at | ENIGMA | NM_005451 | 203370_s_at |
| DSCAML1 | AI433419 | 232059_at | NUCB2 | NM_005013 | 203675_at | DEF6 | NM_022047 | 221293_s_at |
| FLJ90798 | AA131324 | 212419_at | M96 | NM_007358 | 203347_s_at | CD151 | NM_004357 | 204306_s_at |
| ELMO1 | NM_014800 | 204513_s_at | LSM5 | NM_012322 | 202904_s_at | SLC22A1L | NM_002555 | 204981_at |
| PCSK1N | NM_013271 | 218952_at | NASP | NM_002482 | 201970_s_at | EMP1 | BF445047 | 213895_at |
| CKLFSF5 | AI147740 | 230942_at | BRIP1 | BF056791 | 235609_at | EFEMP2 | AB030655 | 209356_x_at |
| OLIG1 | AL355743 | 228170_at | GMPS | NM_003875 | 214431_at | SERPINA1 | NM_000295 | 202833_s_at |
| SRRM2 | BE464483 | 227984_at | E2F7 | AI341146 | 228033_at | C1QTNF1 | AF232905 | 224197_s_at |
| FGF12(3′) | AV707343 | 238521_at | Ect2 | NM_018098 | 219787_s_at | EPAS1 | NM_001430 | 200879_s_at |
| MIR | AA770170 | 221824_s_at | PCNA | NM_002592 | 201202_at | FOSL2 | N36408 | 218880_at |
| GLUD1 | AI339331 | 200946_x_at | KIAA0101 | NM_014736 | 202503_s_at | C1QTNF1 | NM_030968 | 220975_s_at |
| RAP2B | NM_002886 | 214487_s_at | ORC6L | NM_014321 | 219105_x_at | COL4A1 | NM_001845 | 211981_at |
| NONE | BF724210 | 224763_at | CGI-09 | BE897074 | 222768_s_at | SOCS3 | BG035761 | 206359_at |
| NCAM1 | BF348061 | 214952_at | LOC388279 | AA954994 | 238021_s_at | CD164L1 | NM_020404 | 219025_at |
| P2RX7 | NM_002562 | 207091_at | GGH | NM_003878 | 203560_at | ICAM1 | NM_000201 | 202638_s_at |
| FXYD6 | NM_022003 | 217897_at | TRIPIN | AW965339 | 235425_at | FPRL1 | M88107 | 210772_at |
| APOE | NM_000041 | 203382_s_at | KIAA0895 | AB020702 | 213424_at | HSD3B7 | BC004929 | 222817_at |
| PLCB1 | AA393484 | 215687_x_at | FLJ10036 | AA824298 | 222606_at | MGC10204 | AI674922 | 227592_at |
| GRID1 | N48357 | 231977_at | MAGOH | AF067173 | 210093_s_at | EDG2 | AW269335 | 204036_at |
| MAPT | AA199717 | 225379_at | TOP2A | AU159942 | 201291_s_at | BHLHB2 | BG326045 | 201169_s_at |
| TMOD2 | AW207699 | 226186_at | FLJ22028 | AI962943 | 212918_at | PLAUR | U08839 | 210845_s_at |
| PKNOX2 | AK023792 | 222171_s_at | NONE | AA430014 | 228776_at | LOC388538 | BE673775 | 230046_at |
| | | | | | | (3′) | | |
| C6orf159 | AW511485 | 236771_at | FBXO5 | NM_012177 | 218875_s_at | MMP14 | AU149305 | 202827_s_at |
| C20ORF58 | BF593263 | 228017_s_at | TRIPIN | N31731 | 230165_at | NRP2 | AF022859 | 211844_s_at |
| C20orf42 | BF591031 | 229545_at | ASK | NM_006716 | 204244_s_at | UGP2 | AV661152 | 231698_at |
| ARL3 | AF038193 | 213433_at | HCAP-G | NM_022346 | 218663_at | RAB34 | AF322067 | 224710_at |
| NAP1L3 | NM_004538 | 204749_at | SHFM1 | NM_006304 | 202276_at | TUWD12 | NM_003774 | 220442_at |
| MAPT | AI870749 | 203928_x_at | IQGAP3 | AW271106 | 229490_s_at | IFITM2 | NM_006435 | 201315_x_at |
| THRA | M24899 | 35846_at | FLJ23311 | NM_024680 | 219990_at | SLC12A9 | BC000154 | 223994_s_at |
| NDRG2 | NM_016250 | 206453_s_at | FOXD4L1 | NM_020667 | 220175_s_at | COL4A2 | X05610 | 211964_at |
| C14orf59 | AI363193 | 229875_at | PRIM2A | NM_000947 | 205628_at | LOC284207 | BG231494 | 225955_at |
| GLUD2 | AC006144 | 215794_x_at | HMMR | NM_012485 | 207165_at | PTRF | AF312393 | 208790_s_at |
| EHD3 | NM_014600 | 218935_at | CDCA7 | AY029179 | 224428_s_at | ITGA3 | NM_002204 | 201474_s_at |
| VVSM32627 | AU145402 | 232192_at | MGC5576 | NM_024056 | 201764_at | PLAUR | AY029180 | 211924_s_at |
| APOE | N33009 | 203381_s_at | ZF | NM_021212 | 202979_s_at | FAM20C | BE874872 | 226722_at |
| GALNT13 | AC009227 | 234472_at | HEC | NM_006101 | 204162_at | KIAA0963 | AC005390 | 215760_s_at |
| GNAL | AI082827 | 214071_at | MGC14126 | AW665748 | 230860_at | SLC39A8 | NM_022154 | 219869_s_at |
| | | | (3′) | | | | | |
| FLJ37659 | AA909330 | 231131_at | DKFZP434G2226 | NM_031217 | 221258_s_at | FLNA | AI625550 | 214752_x_at |
| FAIM2 | NM_012306 | 203619_s_at | RAMP | NM_016448 | 218585_s_at | THADA | AI674059 | 238938_at |
| | | | | | | (intron) | | |
| C10orf30 | AW195407 | 227341_at | TOM1L1 | NM_005486 | 204485_s_at | EFNB2 | U16797 | 202669_s_at |
| RGC32 | NM_014059 | 218723_s_at | HELLS(3′) | AI889959 | 227350_at | NRP1 | AF145712 | 210510_s_at |
| MAPT | J03778 | 206401_s_at | STK18 | AL043646 | 204886_at | HOMER3 | AC002985 | 222222_s_at |
| MAPT | AI056359 | 203929_s_at | NJMU-R1 | NM_022344 | 203830_at | FLJ25348 | BF689253 | 235417_at |
| GRIK4 | NM_014619 | 208552_at | CCNB1 | N90191 | 228729_at | RUNX1 | D43968 | 209360_s_at |
| MGC20785 | AW173080 | 241729_at | GAJ | AY028916 | 223700_at | ACTN1 | BC003576 | 208637_x_at |
| PLCB1 | AL049593 | 213222_at | TCF19 | BC002493 | 223274_at | ACTN1 | M95178 | 211160_x_at |
| ADAM22 | AW242701 | 213411_at | ABCA5 | BF693921 | 213353_at | TNFRSF10D | AI738556 | 227345_at |
| DLL3 | NM_016941 | 219537_x_at | T2BP | AA195074 | 226117_at | IFITM3 | BF338947 | 212203_x_at |
| TPCN2 | AL137479 | 231978_at | HCAP-G | NM_022346 | 218662_s_at | OSBPL3 | AI202969 | 209626_s_at |
| ABLIM1 | NM_006720 | 200965_s_at | EZH2 | NM_004456 | 203358_s_at | SERPINE1 | AL574210 | 202627_s_at |
| NONE | BE223030 | 213841_at | SIP | AF275803 | 210691_s_at | FLNA | AW051856 | 213746_s_at |
| NONE | N47328 | 239509_at | WEE1 | X62048 | 212533_at | MYL9 | NM_006097 | 201058_s_at |
| PDE2A | NM_002599 | 204134_at | CHEK1 | AA224205 | 238075_at | PVRL2 | BE867789 | 232078_at |
| EPB41L2 | BF511685 | 201718_s_at | FSHPRH1 | BF793446 | 214804_at | T1A-2 | AW590196 | 226658_at |
| C20ORF58 | BF593263 | 228018_at | FLJ10706 | NM_018186 | 220840_s_at | BACE2 | NM_012105 | 217867_x_at |
| ARPP-21 | AL133109 | 231935_at | LOC388279 | AA954994 | 238022_at | MVP | NM_017458 | 202180_s_at |
| KLRC3 | NM_002261 | 207723_s_at | Stk6 | NM_003158 | 208079_s_at | FAM20C | AK026140 | 229438_at |
| AF038169 | AW665278 | 230336_at | FLJ20060 | NM_017645 | 218602_s_at | CD97 | NM_001784 | 202910_s_at |
| (intron) | | | | | | | | |
| HBT8 | BG413612 | 226591_at | MGC12458 | BC002836 | 211200_s_at | TAGLN | NM_003186 | 205547_s_at |
| ABHD6 | AF225418 | 221679_s_at | ZNF367 | N62196 | 229551_x_at | SGSH | NM_000199 | 204293_at |
| SOX6 | AF309034 | 223865_at | NONE | AA969238 | 227793_at | T1A-2 | NM_006474 | 204879_at |
| NONE | AV724769 | 235118_at | FLJ10292 | NM_018048 | 218894_s_at | ECE1 | NM_001397 | 201750_s_at |
| RRP22 | NM_006477 | 206850_at | BARD1 | NM_000465 | 205345_at | GARP | NM_005512 | 203835_at |
| DLL3 | BE350882 | 222898_s_at | TMEM14B | BC001033 | 223133_at | PLEKHF1 | NM_024310 | 219566_at |
| PIPPIN | AL023553 | 209981_at | MGC14801 | BC005997 | 224443_at | LIF | NM_002309 | 205266_at |
| EPHB1 | AF037333 | 211898_s_at | ABHD3 | AL534702 | 213017_at | TSAP6 | NM_018234 | 218424_s_at |
| NONE | BF433749 | 241255_at | TTK | NM_003318 | 204822_at | TNC | NM_002160 | 201645_at |
| MGC11279 | NM_024326 | 218938_at | KIF14 | AW183154 | 236641_at | SALL4 | NM_020436 | 229661_at |
| PARD3 | AF196185 | 221527_s_at | NPHP1 | BF216535 | 238843_at | PAPPA | BG434272 | 224942_at |
| FLJ20581 | AI733019 | 89977_at | FLJ20105 | NM_017669 | 219650_at | FGG | AI133452 | 226621_at |
| dA201G10.1 | AF070565 | 232833_at | KNTC1 | NM_014708 | 206316_s_at | ANGPTL4 | AF169312 | 223333_s_at |
| SNAP91 | NM_014841 | 204953_at | MAD2L1 | NM_002358 | 203362_s_at | ANGPT2 | AF187858 | 211148_s_at |
| GABBR1 | NM_001470 | 203146_s_at | CDC6 | U77949 | 203967_at | ACTA2 | NM_001613 | 200974_at |
| NONE | H12055 | 240869_at | FLJ20641 | NM_017915 | 220060_s_at | ICAM1 | AI608725 | 202637_s_at |
| B3GAT1 | NM_018644 | 219521_at | ANAPC7 | NM_004856 | 204709_s_at | PAPPA | AU156721 | 232748_at |
| | | | | | | (intron) | | |
| NONE | N63005 | 236576_at | RFC4 | NM_002916 | 204023_at | WARP | AW292148 | 222723_at |
| FAM13C1 | U79304 | 214914_at | CCNE2 | NM_004702 | 205034_at | ARHJ | AI583530 | 235489_at |
| APOE | AI358867 | 212884_x_at | EIF1A | BE542684 | 201016_at | ZYX | NM_003461 | 200808_s_at |
| NONE | BF724178 | 229613_at | MGC20486 | BG338983 | 224715_at | FES | NM_002005 | 205418_at |
| GAB2 | NM_012296 | 203853_s_at | SIP1 | NM_003616 | 205063_at | ITGA1 | X68742 | 214660_at |
| RTN1 | BC000314 | 210222_s_at | MELK | NM_014791 | 204825_at | FOSL2 | AI670862 | 225262_at |
| SOX6 | AI480314 | 227498_at | LOC115106 | AV715391 | 225297_at | FLJ14464 | AL040631 | 226401_at |
| MCF2 | NM_005369 | 208017_s_at | TTC12 | NM_017868 | 219587_at | EHD2 | AI417917 | 221870_at |
| GPR27 | AI703476 | 227769_at | CCDC2 | NM_025103 | 219174_at | PDCD1LG1 | AI608902 | 227458_at |
| NDRG2 | W74452 | 214279_s_at | POLG | W74442 | 213007_at | LOC255783 | AW172584 | 227325_at |
| MPPE1 | BF476502 | 213924_at | KIAA0912 | AI130715 | 239413_at | ITGA7 | AF072132 | 209663_s_at |
| BCAN | AF229053 | 221623_at | FLJ10493 | NM_018112 | 218772_x_at | UNC93B1 | AW001274 | 225869_s_at |
| RGC32 | AI744499 | 228193_s_at | FLJ32745 | BF213953 | 235644_at | ANPEP | NM_001150 | 202888_s_at |
| NONE | H39185 | 240433_x_at | PIN4 | BE674061 | 214224_s_at | ShrmL | AB040914 | 225548_at |
| PTGDS | NM_000954 | 212187_x_at | SMC2L1 | NM_006444 | 204240_s_at | PLAU | K03226 | 211668_s_at |
| KCNB1 | L02840 | 211006_s_at | KIAA0101 | BC005832 | 211713_x_at | BCL3 | NM_005178 | 204908_s_at |
| NONE | AI984607 | 227425_at | SMC4L1 | NM_005496 | 201663_s_at | FLT1 | U01134 | 210287_s_at |
| KIAA0820 | AL136712 | 209839_at | KLIP1 | NM_024629 | 218883_s_at | MSCP | BG251467 | 222528_s_at |
| SCG3 | NM_013243 | 219196_at | FLJ10036 | NM_017975 | 218349_s_at | PP2447 | BG399562 | 221807_s_at |
| SNRPN | AU118874 | 214834_at | dJ383J4.3 | N29457 | 232065_x_at | PYGO2 | AI091079 | 214853_s_at |
| SLC18A2 | AI890972 | 213549_at | PPIL5 | AA742244 | 235113_at | PI3 | L10343 | 41469_at |
| HSPA12A | AB007877 | 214434_at | ACYP1 | NM_001107 | 205260_s_at | EDG2 | BF055366 | 204037_at |
| 13CDNA73 | NM_023037 | 204072_s_at | XRCC4 | AB017445 | 205071_x_at | SERPINE1 | NM_000602 | 202628_s_at |
| NTRK2 | BE858459 | 236095_at | CHNL28862 | AW293376 | 237585_at | MYO1D | AI821121 | 241645_at |
| | | | | | | (intron) | | |
| C20orf42 | AA469071 | 60474_at | CDKN2C | NM_001262 | 204159_at | RUNX1 | L34598 | 209359_x_at |
| RIMS2 | NM_014677 | 206137_at | PPIC | BE962749 | 204517_at | GGN | AA421493 | 231420_at |
| OVOL1 | NM_004561 | 206604_at | DKFZp762E1312 | NM_018410 | 218726_at | PDGFRL | NM_006207 | 205226_at |
| KIAA1244 | AI979261 | 228051_at | MNS1 | NM_018365 | 219703_at | COL4A1 | AI922605 | 211980_at |
| ALDOC | NM_005165 | 202022_at | CHNL28862 | AA625683 | 236915_at | NONE | BE045384 | 230501_at |
| MN1 | NM_002430 | 205330_at | MGC13102 | BC005094 | 223544_at | PLA2G5 | AL158172 | 215870_s_at |
| LOC338645 | X81895 | 215323_at | KIF14 | NM_014875 | 206364_at | ANGPT2 | NM_001147 | 205572_at |
| SORBS1 | AF136381 | 211819_s_at | PAWR | NM_002583 | 204005_s_at | SERPINH1 | NM_004353 | 207714_s_at |
| C20orf42 | NM_017671 | 218796_at | TMPO | AL566034 | 224944_at | KLF16 | BF590630 | 226328_at |
| RAB6B | AW118072 | 221792_at | TOP2A | AL561834 | 201292_at | FLT1 | AA149648 | 226498_at |
| MAPK8IP2 | NM_012324 | 205050_s_at | CGI-09 | AB032979 | 233970_s_at | FLJ20699 | NM_017931 | 218272_at |
| ALDH5A1 | NM_001080 | 203609_s_at | NONE | AW262022 | 232242_at | SOCS3 | AI244908 | 227697_at |
| LRRC4 | AF196976 | 223552_at | IL13RA2 | NM_000640 | 206172_at | SERPINA1 | AF119873 | 211429_s_at |
| NONE | N50714 | 236038_at | MLF1 | NM_022443 | 204784_s_at | THBD | NM_000361 | 203888_at |
| NUMA1 | AI337584 | 214251_s_at | FBXO5 | AK026197 | 234863_x_at | LOC56926 | AA781143 | 222206_s_at |
| ID4 | U16153 | 209293_x_at | CHAF1B | NM_005441 | 204775_at | FLNA | NM_001456 | 200859_x_at |
| FLJ20581 | NM_017888 | 220061_at | CHEK1 | NM_001274 | 205394_at | ITGA7 | AI827972 | 227055_at |
| THRA | NM_003250 | 204100_at | LOC51668 | NM_016126 | 203960_s_at | RRBP1 | AA706065 | 201204_s_at |
| WNT7B | BE736994 | 217681_at | PPIG | U40763 | 208995_s_at | RYR3 | NM_001036 | 206306_at |
| DLGAP1 | U55983 | 235527_at | Stk6 | NM_003600 | 204092_s_at | PI3 | NM_002638 | 203691_at |
| PLEKHB1 | AF081583 | 209504_s_at | PRPS2 | NM_002765 | 203401_at | PLA2G5 | NM_000929 | 206178_at |
| C20ORF58 | AI569665 | 230771_at | TRIM36 | NM_018700 | 219736_at | HK3 | NM_002115 | 205936_s_at |
| PSD | NM_002779 | 208102_s_at | LMNB1 | NM_005573 | 203276_at | LZTS1 | NM_021020 | 219042_at |
| SMOC1 | BF516292 | 222783_s_at | NEK2 | NM_002497 | 204641_at | PDGFA | NM_002607 | 205463_s_at |
| NTRK2 | R39159 | 229463_at | MGC14289 | AI188445 | 228280_at | GPR116 | N95226 | 212951_at |
| OLIG2 | AA757419 | 213825_at | RRM2 | BE966236 | 201890_at | THBD | NM_000361 | 203887_s_at |
| SH3GL2 | NM_003026 | 205751_at | PAWR | AI189509 | 226231_at | RIL | BC003096 | 211564_s_at |
| DLL3 | AW341182 | 230568_x_at | ARNTL2 | AF231339 | 224204_x_at | PTK9 | AA827894 | 243033_at |
| CRF | NM_006688 | 205575_at | ASPM | NM_018123 | 219918_s_at | C1RL | NM_016546 | 218983_at |
| MGC20785 | BE552411 | 236290_at | PAWR | AI091432 | 226223_at | TRIM47 | AW249467 | 225868_at |
| KIF5A | NM_004984 | 205318_at | RPA3 | BC005264 | 209507_at | FBXL9 | AF176701 | 232545_at |
| GRIA2 | NM_000826 | 205358_at | CCNB1 | BE407516 | 214710_s_at | LOC151300 | AI125183 | 239507_at |
| ASRG13336 | AW300083 | 236287_at | PAIP1 | NM_006451 | 208051_s_at | FCGR2A | U90939 | 210992_x_at |
| SCD | AF132203 | 223839_s_at | ANKRD5 | NM_022096 | 220144_s_at | KIAA0233 | NM_014745 | 202771_at |
| NONE | AW242720 | 227550_at | NONE | AI220427 | 230696_at | JUNB | NM_002229 | 201473_at |
| RTN1 | NM_021136 | 203485_at | EMP2 | AV686514 | 225078_at | PML | BC000080 | 211012_s_at |
| OLIG2 | AI870776 | 213824_at | LOC400802 | BF508679 | 230121_at | SLC16A3 | NM_004207 | 202856_s_at |
| GRIA4 | H20055 | 238663_x_at | XM_377845 | AI822134 | 238865_at | ITGA7 | AK022548 | 216331_at |
| C5orf12 | AI700633 | 212812_at | ACN9 | NM_020186 | 218981_at | FLJ43339 | BE673226 | 227272_at |
| (intron) | | | | | | | | |
| SATB1(3′) | AA002140 | 241365_at | MCM2 | NM_004526 | 202107_s_at | CA12 | BC000278 | 210735_s_at |
| SLC25A21 | AA770060 | 230307_at | RAD51 | D14134 | 205023_at | ESM1 | NM_007036 | 208394_x_at |
| LOC91752 | AF052145 | 215767_at | CHEK1 | NM_001274 | 205393_s_at | FBN1 | AI264196 | 202765_s_at |
| CALCRL | AI478743 | 234996_at | CDKN2A | NM_000077 | 207039_at | NK4 | NM_004221 | 203828_s_at |
| KIAA0843 | NM_014945 | 205730_s_at | FANCD2 | AA579890 | 242560_at | B4GALT1 | AV687517 | 228498_at |
| CALN1 | AF282250 | 223885_at | TIMELESS | NM_003920 | 203046_s_at | C1R | AL573058 | 212067_s_at |
| TMLHE | NM_018196 | 218790_s_at | GCLM | NM_002061 | 203925_at | CECR2 | AB051527 | 233695_s_at |
| LOC255426 | AI828026 | 236748_at | FLJ12571 | BF664545 | 235949_at | | | |
| | | | (3′) | | | | | |
| FGF14 | NM_004115 | 221310_at | KIF4A | NM_012310 | 218355_at | | | |
| EPHB1 | AF037334 | 210753_s_at | CDCA1 | AF326731 | 223381_at | | | |
| ARRB1(3′) | AL157484 | 221861_at | CHAF1A | NM_005483 | 203976_s_at | | | |
| C19orf4 | NM_012109 | 219005_at | FLJ12571 | NM_024926 | 219758_at | | | |
| HBT8 | BE783065 | 226587_at | Pfs2 | BC003186 | 221521_s_at | | | |
| BMP2 | NM_001200 | 205290_s_at | FLJ90806 | AI469788 | 235572_at | | | |
| PACE4 | NM_002570 | 207414_s_at | NUSAP1 | NM_018454 | 219978_s_at | | | |
| KIAA0937 | AV728526 | 212611_at | DHFR | BC003584 | 202533_s_at | | | |
| FLJ20701 | NM_017933 | 219093_at | APG12L | NM_004707 | 204833_at | | | |
| NONE | BF224377 | 231214_at | HT021 | NM_020685 | 219288_at | | | |
| NONE | AL390170 | 213904_at | ZWINT | NM_007057 | 204026_s_at | | | |
| SCD | BC005807 | 211708_s_at | BUB1 | AF043294 | 209642_at | | | |
| KIF5A | BF196255 | 229921_at | DLG7 | NM_014750 | 203764_at | | | |
| DKFZp761D112 | AL136588 | 223614_at | AAT1 | N62817 | 236222_at | | | |
| CSMD3 | AI187364 | 240228_at | LOC161577 | AA020920 | 243198_at | | | |
| NONE | AI692426 | 230551_at | PDK1 | AU146532 | 226452_at | | | |
| SLIT1 | AB011537 | 213601_at | CKS2 | NM_001827 | 204170_s_at | | | |
| ADCY2 | AU149572 | 213217_at | HIC | AF054589 | 211675_s_at | | | |
| DPP10 | AL538781 | 228598_at | DKFZp586G0123 | NM_013386 | 204342_at | | | |
| Nrg3 | H05240 | 229233_at | CENPA | NM_001809 | 204962_s_at | | | |
| SLNL4764 | AA017721 | 214046_at | CDC2 | AL524035 | 203213_at | | | |
| DSCAM(3′) | BF941609 | 240218_at | GAS41 | NM_006530 | 218911_at | | | |
| DSCAM | AF023450 | 211484_s_at | PEG10 | AL582836 | 212094_at | | | |
| FSD1 | NM_024333 | 219170_at | LOC139886 | AU145277 | 228654_at | | | |
| PTGDS | BC005939 | 211748_x_at | EVC2 | AA234305 | 229974_at | | | |
| JPH3 | NM_020655 | 220188_at | BRRN1 | D38553 | 212949_at | | | |
| NONE | BF345233 | 228679_at | ABCA5 | AI568925 | 241705_at | | | |
| ASCL1 | BC002341 | 209987_s_at | CDKN2A | U38945 | 209644_x_at | | | |
| PLCB1 | AY004175 | 211925_s_at | HMMR | U29343 | 209709_s_at | | | |
| MAPK10(3′) | AI263044 | 214376_at | CENPE | NM_001813 | 205046_at | | | |
| UNQ470 | AL541276 | 228403_at | E2F1 | NM_005225 | 204947_at | | | |
| LOC340554 | AW007160 | 229234_at | TOP2A | T96523 | 237469_at | | | |
| | | | (intron) | | | | | |
| GNAO1 | BE670563 | 204762_s_at | CDC6 | NM_001254 | 203968_s_at | | | |
| GAD1 | NM_000817 | 205278_at | BCAA | BG492359 | 226936_at | | | |
| CLONE25003 | N66656 | 229655_at | SIL | NM_003035 | 205339_at | | | |
| RhoGAP2 | NM_021226 | 206298_at | RNPC6 | AI677701 | 235004_at | | | |
| FGF12 | D60438 | 240067_at | FLJ12973 | AI220472 | 239680_at | | | |
| (intron) | | | | | | | | |
| GRIA2 | BE219628 | 236538_at | FLJ90440 | AI627704 | 226908_at | | | |
| REPRIMO | NM_019845 | 219370_at | CDC25C | NM_001790 | 205167_s_at | | | |
| KIAA0861(3′) | AI186173 | 242651_at | KLIP1 | AA460299 | 229305_at | | | |
| ARRB1(3′) | N80935 | 49111_at | CENPF | U30872 | 209172_s_at | | | |
| ARRB1(3′) | AI201594 | 43511_s_at | FBXO11 | NM_025133 | 219208_at | | | |
| GPR49 | AL524520 | 213880_at | MGC14801 | BC005997 | 224444_s_at | | | |
| GNAL | R20102 | 206355_at | ITGA2 | N95414 | 227314_at | | | |
| TPM1 | NM_000366 | 206117_at | BM039 | NM_018455 | 219555_s_at | | | |
| GPR51 | AF069755 | 209991_x_at | SHOX2 | NM_006884 | 208443_x_at | | | |
| MGC20785 | AL120332 | 231980_at | | | | | | |
| NONE | AA706282 | 244184_at | | | | | | |
| UBE1C | BE221817 | 229831_at | | | | | | |
| DKFZp547D2210 | AI186464 | 228165_at | | | | | | |
| TRIM31 | X81006 | 215444_s_at | | | | | | |
| EB-1 | AW005572 | 227440_at | | | | | | |
| NEU4 | AK025617 | 222957_at | | | | | | |
| NONE | AW450397 | 236433_at | | | | | | |
| CECR6 | AF307451 | 224393_s_at | | | | | | |
| LOC286097 | AI868167 | 238458_at | | | | | | |
| FLJ10970 | NM_018286 | 219230_at | | | | | | |
| RPIP8 | NM_006695 | 206196_s_at | | | | | | |
| RGC32 | BG542501 | 239827_at | | | | | | |
| SSTR1 | R62424 | 235591_at | | | | | | |
| SLC1A1 | AW235061 | 213664_at | | | | | | |
| KIAA0527 | BF977837 | 214954_at | | | | | | |
| FLJ20300 | NM_017753 | 219732_at | | | | | | |
| DLL1 | AF196571 | 224215_s_at | | | | | | |
| SALF(intron) | AL049443 | 215306_at | | | | | | |
| GPR86 | NM_023914 | 220005_at | | | | | | |
| GPR158 | R41459 | 232195_at | | | | | | |
| C20orf19 | NM_018474 | 219961_s_at | | | | | | |
| VVSM32627(3′) | R39126 | 230932_at | | | | | | |
| CNTN1 | U07820 | 211203_s_at | | | | | | |
| NTN4 | AF278532 | 223315_at | | | | | | |
| PDK4 | NM_002612 | 205960_at | | | | | | |
| CDR1 | NM_004065 | 207276_at | | | | | | |
| NET1 | NM_005863 | 201830_s_at | | | | | | |
| SHD | AW452918 | 227845_s_at | | | | | | |
| LOC254559 | AI611973 | 238603_at | | | | | | |
| SLC1A4 | AB026689 | 209611_s_at | | | | | | |
| HMGA2 | BF435376 | 213486_at | | | | | | |
| CLONE25003 | AI953086 | 237094_at | | | | | | |
| RAB11-FIP4 | BC006240 | 224482_s_at | | | | | | |
| SLC1A4 | BF340083 | 209610_s_at | | | | | | |
| JPH3 | AL537395 | 229294_at | | | | | | |
| GNAL | NM_002071 | 206356_s_at | | | | | | |
| KIAA1796 | AW014022 | 230577_at | | | | | | |
| GABRA3 | NM_000808 | 207210_at | | | | | | |
| SCN3A | AF225986 | 210432_s_at | | | | | | |
| F2 | NM_000506 | 205754_at | | | | | | |
| DKFZp566D234 | AA129444 | 232010_at | | | | | | |
| HIP1R | AB013384 | 209558_s_at | | | | | | |
| SCD | AA678241 | 200831_s_at | | | | | | |
| NONE | BF435290 | 240578_at | | | | | | |
| SSTR2 | AF184174 | 217455_s_at | | | | | | |
| DTVS31377(3′) | AW451455 | 229612_at | | | | | | |
| CALCRL | U17473 | 210815_s_at | | | | | | |
| ASCL1 | BC001638 | 209988_s_at | | | | | | |
| PRKCZ | NM_002744 | 202178_at | | | | | | |
| KIAA0534 | AI817331 | 213744_at | | | | | | |
| C10orf24 | N53051 | 229378_at | | | | | | |
| PNUTL2 | U88870 | 210657_s_at | | | | | | |
| BMP2 | AA583044 | 205289_at | | | | | | |
| GRIA1 | M64752 | 211520_s_at | | | | | | |
| GPR51 | AF095784 | 211679_x_at | | | | | | |
| HEY2 | NM_012259 | 219743_at | | | | | | |
| SMOC1 | AJ249900 | 222784_at | | | | | | |
| SLC25A15P | BF000009 | 243952_at | | | | | | |
| MADH9 | NM_005905 | 206320_s_at | | | | | | |
| KCTD4 | H10766 | 240512_x_at | | | | | | |
| BCAN | AI205180 | 91920_at | | | | | | |
| LOC349136 | AI968904 | 243526_at | | | | | | |
| RGS9 | NM_003835 | 206518_s_at | | | | | | |
| NONE | AF339807 | 215469_at | | | | | | |
| GABRB3 | AI693153 | 229724_at | | | | | | |
| VMP | R38624 | 239293_at | | | | | | |
| MECT1 | AB014516 | 213091_at | | | | | | |
| TAL1 | X51990 | 216925_s_at | | | | | | |
| GPR51 | AF095723 | 217077_s_at | | | | | | |
| NONE | AI656867 | 231103_at | | | | | | |
| SLITRK2 | AL109653 | 233051_at | | | | | | |
| KIF21B | NM_017596 | 204411_at | | | | | | |
| KCNN3 | NM_002249 | 205903_s_at | | | | | | |
| SGCG | NM_000231 | 207302_at | | | | | | |
| RAC3 | NM_005052 | 206103_at | | | | | | |
| ZNF488 | AI056483 | 229901_at | | | | | | |
| SORCS3 | AB028982 | 215522_at | | | | | | |
| KIAA1941 | BE222282 | 230287_at | | | | | | |
| FGF13 | NM_004114 | 205110_s_at | | | | | | |
| PKP4 | AL050364 | 214874_at | | | | | | |
| REPS2(3′) | AW962020 | 242571_at | | | | | | |
| NONE | BF513800 | 244623_at | | | | | | |
| OPCML | AF070577 | 214111_at | | | | | | |
| CNTN1 | AW072790 | 227202_at | | | | | | |
| NONE | AI374686 | 244218_at | | | | | | |
| Maf | BF508646 | 209347_s_at | | | | | | |
| SLITRK5 | AW449813 | 214930_at | | | | | | |
| SCAPIN1 | AL357503 | 227949_at | | | | | | |
| HS3ST4 | AF105378 | 228206_at | | | | | | |
| alpha-1,3- | AI972498 | 228376_at | | | | | | |
| galactosyl- | | | | | | | | |
| transferase | | | | | | | | |
| type1 | | | | | | | | |
| NONE | AW269887 | 236333_at | | | | | | |
| FBXO2 | NM_012168 | 219305_x_at | | | | | | |
| GPRC5B | AF202640 | 203631_s_at | | | | | | |
| MGC39325 | BE672313 | 221959_at | | | | | | |
| SEC31L2 | AF274863 | 209889_at | | | | | | |
| CCNK | NM_003858 | 219273_at | | | | | | |
| KLRC3 | NM_002260 | 206785_s_at | | | | | | |
| AKR1C1 | S68290 | 216594_x_at | | | | | | |
| ATSV | AL533416 | 225482_at | | | | | | |
| PDK2 | AI870615 | 213724_s_at | | | | | | |
| CLONE25003 | AV723914 | 229459_at | | | | | | |
| NOG | AL575177 | 231798_at | | | | | | |
| SHREW1 | AA835004 | 215789_s_at | | | | | | |
| RASSF4 | AF260335 | 221578_at | | | | | | |
| BCAN | NM_021948 | 219107_at | | | | | | |
| IL17D | BE856748 | 227401_at | | | | | | |
| USH1C | AB006955 | 211184_s_at | | | | | | |
| NTRK2 | BF674712 | 214680_at | | | | | | |
| RPL5 | U66589 | 210035_s_at | | | | | | |
| SEZ6L | BE672217 | 231650_s_at | | | | | | |
| TTYH1 | NM_020659 | 219415_at | | | | | | |
| PTGDS | M61900 | 211663_x_at | | | | | | |
| KIAA1713(3′) | AF070541 | 214162_at | | | | | | |
| HLF | W60800 | 204754_at | | | | | | |
| TIMP4 | NM_003256 | 206243_at | | | | | | |
| KCTD4 | N52767 | 239787_at | | | | | | |
| TNKS2 | H03262 | 241909_at | | | | | | |
| LOC387944(3′) | H09780 | 230869_at | | | | | | |
| SCD | AF116616 | 211162_x_at | | | | | | |

A “native sequence GDM polypeptide” comprises a polypeptide having the same amino acid sequence as the corresponding GDM polypeptide derived from nature. Such native sequence GDM polypeptides can be isolated from nature or can be produced by recombinant or synthetic means. The term “native sequence GDM polypeptide” specifically encompasses naturally-occurring truncated or secreted forms of the specific GDM polypeptide (e.g., an extracellular domain sequence), naturally-occurring variant forms (e.g., alternatively spliced forms) and naturally-occurring allelic variants of the polypeptide. In certain embodiments of the invention, the native sequence GDM polypeptides disclosed herein are mature or full-length native sequence polypeptides corresponding to the polypeptides recited in Table A. “GDM polypeptide variant” means a GDM polypeptide, preferably active forms thereof, as defined herein, having at least about 80% amino acid sequence identity with a full-length native sequence GDM polypeptide sequence, as disclosed herein, and variant forms thereof lacking the signal peptide, an extracellular domain, or any other fragment of a full length native sequence GDM polyeptide polypeptide such as those referenced herein. Such variant polypeptides include, for instance, polypeptides wherein one or more amino acid residues are added, or deleted, at the N- or C-terminus of the full-length native amino acid sequence. In a specific aspect, such variant polypeptides will have at least about 80% amino acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% amino acid sequence identity, to a full-length native sequence GDM polypeptide sequence polypeptide, as disclosed herein, and variant forms thereof lacking the signal peptide, an extracellular domain, or any other fragment of a full length native sequence GDM polypeptide polypeptide such as those disclosed herein. In a specific aspect, such variant polypeptides will vary at least about 1, 2, 3, 4, 5, 6, 7, 8, 9, 10, 20, 25, 30, 35, 40, 45, 50, 60, 70, 80, 90, 100, 125, 150, 200, 250, 300 or more amino acid residues in length from the corresponding native sequence polypeptide. Alternatively, such variant polypeptides will have no more than one conservative amino acid substitution as compared to the corresponding native polypeptide sequence, alternatively no more than 2, 3, 4, 5, 6, 7, 8, 9, or 10 conservative amino acid substitution as compared to the native polypeptide sequence.

“Percent (%) amino acid sequence identity” with respect to the GDM polypeptide sequences identified herein is defined as the percentage of amino acid residues in a candidate sequence that are identical with the amino acid residues in the specific GDM polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. For purposes herein, however, % amino acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

“GDM variant polynucleotide” or “GDM variant nucleic acid sequence” means a nucleic acid molecule which encodes a GDM polypeptide, preferably active forms thereof, as defined herein, and which have at least about 80% nucleic acid sequence identity with a nucleotide acid sequence encoding a full-length native sequence GDM polypeptide sequence identified herein, or any other fragment of the respective full-length GDM polypeptide sequence as identified herein (such as those encoded by a nucleic acid that represents only a portion of the complete coding sequence for a full-length GDM polypeptide). Ordinarily, such variant polynucleotides will have at least about 80% nucleic acid sequence identity, alternatively at least about 81%, 82%, 83%, 84%, 85%, 86%, 87%, 88%, 89%, 90%, 91%, 92%, 93%, 94%, 95%, 96%, 97%, 98%, or 99% nucleic acid sequence identity with a nucleic acid sequence encoding the respective full-length native sequence GDM polypeptide sequence or any other fragment of the respective full-length GDM polypeptide sequence identified herein. Such variant polynucleotides do not encompass the native nucleotide sequence.

Ordinarily, such variant polynucleotides vary at least about 50 nucleotides in length from the native sequence polypeptide, alternatively the variance can be at least about 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

“Percent (%) nucleic acid sequence identity” with respect to GDM polypeptide-encoding nucleic acid sequences identified herein is defined as the percentage of nucleotides in a candidate sequence that are identical with the nucleotides in the GDM nucleic acid sequence of interest, respectively, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN or Megalign (DNASTAR) software. For purposes herein, however, % nucleic acid sequence identity values are generated using the sequence comparison computer program ALIGN-2, wherein the complete source code for the ALIGN-2 program is provided in Table 1 below. The ALIGN-2 sequence comparison computer program was authored by Genentech, Inc. and the source code shown in Table 1 below has been filed with user documentation in the U.S. Copyright Office, Washington D.C., 20559, where it is registered under U.S. Copyright Registration No. TXU510087. The ALIGN-2 program is publicly available through Genentech, Inc., South San Francisco, Calif. or may be compiled from the source code provided in Table 1 below. The ALIGN-2 program should be compiled for use on a UNIX operating system, preferably digital UNIX V4.0D. All sequence comparison parameters are set by the ALIGN-2 program and do not vary.

In situations where ALIGN-2 is employed for nucleic acid sequence comparisons, the % nucleic acid sequence identity of a given nucleic acid sequence C to, with, or against a given nucleic acid sequence D (which can alternatively be phrased as a given nucleic acid sequence C that has or comprises a certain % nucleic acid sequence identity to, with, or against a given nucleic acid sequence D) is calculated as follows:
100 times the fraction W/Z

where W is the number of nucleotides scored as identical matches by the sequence alignment program ALIGN-2 in that program's alignment of C and D, and where Z is the total number of nucleotides in D. It will be appreciated that where the length of nucleic acid sequence C is not equal to the length of nucleic acid sequence D, the % nucleic acid sequence identity of C to D will not equal the % nucleic acid sequence identity of D to C. As examples of % nucleic acid sequence identity calculations, Tables 4 and 5, demonstrate how to calculate the % nucleic acid sequence identity of the nucleic acid sequence designated “Comparison DNA” to the nucleic acid sequence designated “REF-DNA”, wherein “REF-DNA” represents a hypothetical GDM-encoding nucleic acid sequence of interest, “Comparison DNA” represents the nucleotide sequence of a nucleic acid molecule against which the “REF-DNA” nucleic acid molecule of interest is being compared, and “N”, “L” and “V” each represent different hypothetical nucleotides. Unless specifically stated otherwise, all % nucleic acid sequence identity values used herein are obtained as described in the immediately preceding paragraph using the ALIGN-2 computer program.

In other embodiments, GDM variant polynucleotides are nucleic acid molecules that encode GDM polypeptides, and which are capable of hybridizing, preferably under stringent hybridization and wash conditions, to nucleotide sequences encoding a full-length GDM polypeptide, as disclosed herein. Such variant polypeptides may be those that are encoded by such variant polynucleotides.

“Isolated”, when used to describe the various GDM polypeptides disclosed herein, means polypeptide that has been identified and separated and/or recovered from a component of its natural environment. Contaminant components of its natural environment are materials that would typically interfere with diagnostic or therapeutic uses for the polypeptide, and may include enzymes, hormones, and other proteinaceous or non-proteinaceous solutes. In preferred embodiments, such polypeptides will be purified (1) to a degree sufficient to obtain at least 15 residues of N-terminal or internal amino acid sequence by use of a spinning cup sequenator, or (2) to homogeneity by SDS-PAGE under non-reducing or reducing conditions using Coomassie blue or, preferably, silver stain. Such isolated polypeptides includes the corresponding polypeptides in situ within recombinant cells, since at least one component of the GDM polypeptide from its natural environment will not be present. Ordinarily, however, such isolated polypeptides will be prepared by at least one purification step.

An “isolated” GDM polypeptide-encoding nucleic acid is a nucleic acid molecule that is identified and separated from at least one contaminant nucleic acid molecule with which it is ordinarily associated in the natural source of the polypeptide-encoding nucleic acid. Any of the above such isolated nucleic acid molecule is other than in the form or setting in which it is found in nature. Any such nucleic acid molecules therefore are distinguished from the specific polypeptide-encoding nucleic acid molecule as it exists in natural cells.

The term “control sequences” refers to DNA sequences necessary for the expression of an operably linked coding sequence in a particular host organism. The control sequences that are suitable for prokaryotes, for example, include a promoter, optionally an operator sequence, and a ribosome binding site. Eukaryotic cells are known to utilize promoters, polyadenylation signals, and enhancers.

Nucleic acid is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous. Linking is accomplished by ligation at convenient restriction sites. If such sites do not exist, the synthetic oligonucleotide adaptors or linkers are used in accordance with conventional practice.

“Stringency” of hybridization reactions is readily determinable by one of ordinary skill in the art, and generally is an empirical calculation dependent upon probe length, washing temperature, and salt concentration. In general, longer probes require higher temperatures for proper annealing, while shorter probes need lower temperatures. Hybridization generally depends on the ability of denatured DNA to reanneal when complementary strands are present in an environment below their melting temperature. The higher the degree of desired homology between the probe and hybridizable sequence, the higher the relative temperature which can be used. As a result, it follows that higher relative temperatures would tend to make the reaction conditions more stringent, while lower temperatures less so. For additional details and explanation of stringency of hybridization reactions, see Ausubel et al., Current Protocols in Molecular Biology, Wiley Interscience Publishers, (1995).

“Stringent conditions” or “high stringency conditions”, as defined herein, may be identified by those that: (1) employ low ionic strength and high temperature for washing, for example 0.015 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” may be identified as described by Sambrook et al., Molecular Cloning: A Laboratory Manual., New York: Cold Spring Harbor Press, 1989, and include the use of washing solution and hybridization conditions (e.g., temperature, ionic strength and % SDS) less stringent that those described above. An example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The ordinarily skilled artisan will recognize how to adjust the temperature, ionic strength, etc. as necessary to accommodate factors such as probe length and the like.

The term “epitope tagged” when used herein refers to a chimeric polypeptide comprising a GDM polypeptide or GDM binding agent fused to a “tag polypeptide”. The tag polypeptide has enough residues to provide an epitope against which an antibody can be made, yet is short enough such that it does not interfere with the activity of the polypeptide to which it is fused. The tag polypeptide preferably also is sufficiently unique so that such antibody does not substantially cross-react with other epitopes. Suitable tag polypeptides generally have at least six amino acid residues and usually between about 8 and 50 amino acid residues (preferably, between about 10 and 20 amino acid residues).

“Active” or “activity” for the purposes herein refers to form(s) of a GDM polypeptides which retain a biological and/or an immunological activity of native or naturally-occurring GDM polypeptide, wherein “biological” activity refers to a biological function (either inhibitory or stimulatory) caused by a native or naturally-occurring GDM other than the ability to induce the production of an antibody against an antigenic epitope possessed by a native or naturally-occurring GDM polypeptide, and an “immunological” activity refers to the ability to induce the production of an antibody against an antigenic epitope possessed by a native or naturally-occurring GDM polypeptide. An active GDM polypeptide, as used herein, is an antigen that is differentially expressed, either from a qualitative or quantitative perspective, on a glioma tumor, relative to its expression on similar tissue that is not afflicted with glioma.

The term “antagonist” is used in the broadest sense, and includes any molecule that partially or fully blocks, inhibits, or neutralizes a biological activity of a native GDM polypeptide disclosed herein. Suitable antagonist molecules specifically include antagonist antibodies or antibody fragments, fragments or amino acid sequence variants of native GDM polypeptides, peptides, antisense oligonucleotides, small organic molecules, etc. Methods for identifying GDM antagonists may comprise contacting a GDM polypeptide, including a cell expressing it, with a candidate agonist or antagonist molecule and measuring a detectable change in one or more biological activities normally associated with the GDM polypeptide.

“Treating” or “treatment” or “alleviation” refers to both therapeutic treatment and prophylactic or preventative measures, wherein the object is to prevent or slow down (lessen) the progression of glioma. “Prognosing” refers to the determination or prediction of the probable course and outcome of a glioma tumor. “Diagnosing” refers to the process of identifying or determining the distinguishing characteristics of a glioma tumor. The process of diagnosing is sometimes also expressed as staging or tumor classification based on severity or disease progression.

Subjects in need of treatment, prognosis or diagnosis include those already with glioma as well as those prone to having glioma or those in whom glioma is to be prevented. A subject or mammal is successfully “treated” for a GDM polypeptide-expressing glioma if, according to the method of the present invention, after receiving a therapeutic amount of a GDM antagonist (e.g., PN antagonist, Prolif antagonist or Mes antagonist), the patient shows observable and/or measurable reduction in or absence of one or more of the following: reduction in the number of glioma tumor cells or absence of such cells; reduction in the tumor size; inhibition (i.e., slow to some extent and preferably stop) of glioma tumor cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent such GDM antagonists may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and tests for calcium level and other enzymes to determine the extent of metastasis. CT scans can also be done to look for spread to regions outside of the glia. The invention described herein relating to the process of prognosing, diagnosing and/or treating involves the determination and evaluation of GDM (e.g., PN, Prolif, Mes, Pten and DLL3) amplification and expression.

“Mammal” for purposes of the treatment of, alleviating the symptoms of or diagnosis of a cancer refers to any animal classified as a mammal., including humans, domestic and farm animals, and zoo, sports, or pet animals, such as dogs, cats, cattle, horses, sheep, pigs, goats, rabbits, ferrets, etc. Preferably, the mammal is human.

Administration “in combination with” one or more further therapeutic agents includes simultaneous (concurrent) and consecutive administration in any order.

“Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWLEEN®, polyethylene glycol (PEG), and PLURONICS®.

By “solid phase” or “solid support” is meant a non-aqueous matrix to which a GDM antagonist or GDM binding agent of the present invention can adhere or attach. Examples of solid phases encompassed herein include those formed partially or entirely of glass (e.g., controlled pore glass), polysaccharides (e.g., agarose), polyacrylamides, polystyrene, polyvinyl alcohol and silicones. In certain embodiments, depending on the context, the solid phase can comprise the well of an assay plate; in others it is a purification column (e.g., an affinity chromatography column). This term also includes a discontinuous solid phase of discrete particles, such as those described in U.S. Pat. No. 4,275,149.

A “liposome” is a small vesicle composed of various types of lipids, phospholipids and/or surfactant which is useful for delivery of a drug (such as a GDM antagonist) to a mammal. The components of the liposome are commonly arranged in a bilayer formation, similar to the lipid arrangement of biological membranes.

A “small molecule” or “small organic molecule” is defined herein to have a molecular weight below about 500 Daltons.

An “effective amount” of a GDM antagonist or GDM binding agent is an amount sufficient to carry out a specifically stated purpose. An “effective amount” may be determined empirically and in a routine manner, in relation to the stated purpose.

The term “therapeutically effective amount” refers to a GDM antagonist or other drug effective to “treat” a disease or disorder in a subject or mammal. In the case of glioma, the therapeutically effective amount of the drug may reduce the number of glioma cells; reduce the tumor size; inhibit (i.e., slow to some extent and preferably stop) the infiltration of glioma tumor cells into peripheral tissue or organs; inhibit (i.e., slow to some extent and preferably stop) tumor metastasis; inhibit, to some extent, tumor growth; and/or relieve to some extent one or more of the symptoms associated with glioma. See the definition herein of “treating”. To the extent the drug may prevent growth and/or kill existing cancer cells, it may be cytostatic and/or cytotoxic.

A “growth inhibitory amount” of a GDM antagonist is an amount capable of inhibiting the growth of a cell, especially tumor, e.g., cancer cell, either in vitro or in vivo. For purposes of inhibiting neoplastic cell growth, such an amount may be determined empirically and in a routine manner.

A “cytotoxic amount” of a GDM antagonist is an amount capable of causing the destruction of a cell, especially a glioma cell, e.g., cancer cell, either in vitro or in vivo. For purposes of inhibiting neoplastic cell growth may be determined empirically and in a routine manner.

The term “antibody” is used in the broadest sense and specifically covers, for example, anti-PN, anti-Prolif and anti-Mes GDM monoclonal antibodies (including antagonist and neutralizing antibodies), anti-PN, anti-Prolif and anti-Mes GDM antibody compositions with polyepitopic specificity, polyclonal antibodies, single chain anti-GDM antibodies, multispecific antibodies (e.g., bispecific) and antigen binding fragments (see below) of all of the above enumerated antibodies as long as they exhibit the desired biological or immunological activity. The term “immunoglobulin” (Ig) is used interchangeably with antibody herein.

An “isolated” antibody is one which has been identified and separated and/or recovered from a component of its natural environment. Contaminant components of its natural environment are materials which would interfere with diagnostic or therapeutic uses for the antibody, and may include enzymes, hormones, and other proteinaceous or nonproteinaceous solutes. In preferred embodiments, the antibody will be purified (1) to greater than 95% by weight of antibody as determined by the Lowry method, and most preferably more than 99% by weight, (2) to a degree sufficient to obtain at least 15 residues of N-terminal or internal amino acid sequence by use of a spinning cup sequenator, or (3) to homogeneity by SDS-PAGE under reducing or nonreducing conditions using Coomassie blue or, preferably, silver stain. Isolated antibody includes the antibody in situ within recombinant cells since at least one component of the antibody's natural environment will not be present. Ordinarily, however, isolated antibody will be prepared by at least one purification step.

The basic 4-chain antibody unit is a heterotetrameric glycoprotein composed of two identical light (L) chains and two identical heavy (H) chains (an IgM antibody consists of 5 of the basic heterotetramer unit along with an additional polypeptide called J chain, and therefore contain 10 antigen binding sites, while secreted IgA antibodies can polymerize to form polyvalent assemblages comprising 2-5 of the basic 4-chain units along with J chain). In the case of IgGs, the 4-chain unit is generally about 150,000 daltons. Each L chain is linked to an H chain by one covalent disulfide bond, while the two H chains are linked to each other by one or more disulfide bonds depending on the H chain isotype. Each H and L chain also has regularly spaced intrachain disulfide bridges. Each H chain has at the N-terminus, a variable domain (V_{H}) followed by three constant domains (C_{H}) for each of the α and γ chains and four C_{H} domains for μ and ε isotypes. Each L chain has at the N-terminus, a variable domain (V_{L}) followed by a constant domain (C_{L}) at its other end. The V_{L} is aligned with the V_{H} and the C_{L} is aligned with the first constant dmain of the heavy chain (CH₁). Particular amino acid residues are believed to form an interface between the light chain and heavy chain variable domains. The pairing of a V_{H} and V_{L} together forms a single antigen-binding site. For the structure and properties of the different classes of antibodies, see, e.g., Basic and Clinical Immunology, 8th edition, Daniel P. Stites, Abba I. Terr and Tristram G. Parslow (eds.), Appleton & Lange, Norwalk, Conn., 1994, page 71 and Chapter 6.

The L chain from any vertebrate species can be assigned to one of two clearly distinct types, called kappa and lambda, based on the amino acid sequences of their constant domains. Depending on the amino acid sequence of the constant domain of their heavy chains (C_{H}), immunoglobulins can be assigned to different classes or isotypes. There are five classes of immunoglobulins: IgA, IgD, IgE, IgG, and IgM, having heavy chains designated α, δ, ε, γ, and μ, respectively. The γ and α classes are further divided into subclasses on the basis of relatively minor differences in C_{H} sequence and function, e.g., humans express the following subclasses: IgG1, IgG2, IgG3, IgG4, IgA1, and IgA2.

The term “variable” refers to the fact that certain segments of the variable domains differ extensively in sequence among antibodies. The V domain mediates antigen binding and define specificity of a particular antibody for its particular antigen. However, the variability is not evenly distributed across the approximately 110-amino acid span of the variable domains. Instead, the V regions consist of relatively invariant stretches called framework regions (FRs) of 15-30 amino acids separated by shorter regions of extreme variability called “hypervariable regions” that are each 9-12 amino acids long. The variable domains of native heavy and light chains each comprise four FRs, largely adopting a β-sheet configuration, connected by three hypervariable regions, which form loops connecting, and in some cases forming part of, the β-sheet structure. The hypervariable regions in each chain are held together in close proximity by the FRs and, with the hypervariable regions from the other chain, contribute to the formation of the antigen-binding site of antibodies (see Kabat et al., Sequences of Proteins of Immunological Interest, 5th Ed. Public Health Service, National Institutes of Health, Bethesda, Md. (1991)). The constant domains are not involved directly in binding an antibody to an antigen, but exhibit various effector functions, such as participation of the antibody in antibody dependent cellular cytotoxicity (ADCC).

The term “hypervariable region” when used herein refers to the amino acid residues of an antibody which are responsible for antigen-binding. The hypervariable region generally comprises amino acid residues from a “complementarity determining region” or “CDR” (e.g. around about Kabat residues 24-34 (L1), 50-56 (L2) and 89-97 (L3) in the V_{L}, and around about Kabat residues 31-35B (H1), 50-65 (H2) and 95-102 (H3) in the V_{H} (Kabat et al., Sequences of Proteins of Immunological Interest, 5th Ed. Public Health Service, National Institutes of Health, Bethesda, Md. (1991)) and/or those residues from a “hypervariable loop” (e.g. around about Chothia residues 26-32 (L1), 50-52 (L2) and 91-96 (L3) in the V_{L}, and 26-32 (H1), 52A-55 (H2) and 96-101 (H3) in the V_{H} (Chothia and Lesk J. Mol. Biol. 196:901-917 (1987)).

The term “monoclonal antibody” as used herein refers to an antibody from a population of substantially homogeneous antibodies, i.e., the individual antibodies comprising the population are identical and/or bind the same epitope(s), except for possible variants that may arise during production of the monoclonal antibody, such variants generally being present in minor amounts. Such monoclonal antibody typically includes an antibody comprising a polypeptide sequence that binds a target, wherein the target-binding polypeptide sequence was obtained by a process that includes the selection of a single target binding polypeptide sequence from a plurality of polypeptide sequences. For example, the selection process can be the selection of a unique clone from a plurality of clones, such as a pool of hybridoma clones, phage clones or recombinant DNA clones. It should be understood that the selected target binding sequence can be further altered, for example, to improve affinity for the target, to humanize the target binding sequence, to improve its production in cell culture, to reduce its immunogenicity in vivo, to create a multispecific antibody, etc., and that an antibody comprising the altered target binding sequence is also a monoclonal antibody of this invention. In contrast to polyclonal antibody preparations which typically include different antibodies directed against different determinants (epitopes), each monoclonal antibody of a monoclonal antibody preparation is directed against a single determinant on an antigen. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including, for example, the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N.Y., 1981)), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338(2):299-310 (2004); Lee et al., J. Mol. Biol.340(5):1073-1093 (2004); Fellouse, Pro Nat. Acad. Sci. USA 101(34):12467-12472 (2004); and Lee et al. J. Immunol. Methods 284(1-2): 119-132 (2004), and technologies for producing human or human-like antibodies in animals that have parts or all of the human immunoglobulin loci or genes encoding human immunoglobulin sequences (see, e.g., WO 1998/24893; WO 1996/34096; WO 1996/33735; WO 1991/10741; Jakobovits et al., Proc. Natl. Acad. Sci. USA, 90:2551 (1993); Jakobovits et al., Nature, 362:255-258 (1993); Bruggemann et al., Year in Immuno., 7:33 (1993); U.S. Pat. Nos. 5,545,806; 5,569,825; 5,591,669 (all of GenPharm); 5,545,807; WO 1997/17852; U.S. Pat. Nos. 5,545,807; 5,545,806; 5,569,825; 5,625,126; 5,633,425; and 5,661,016; Marks et al., Bio/Technoloay, 10: 779-783 (1992); Lonberg et al., Nature, 368: 856-859 (1994); Morrison, Nature, 368: 812-813 (1994); Fishwild et al., Nature Biotechnology, 14: 845-851 (1996); Neuberger, Nature Biotechnology, 14: 826 (1996); and Lonberg and Huszar, Intern. Rev. Immunol., 13: 65-93 (1995).

“Chimeric” antibodies (immunoglobulins) have a portion of the heavy and/or light chain identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA 81:6851-6855 (1984)). Humanized antibody as used herein is a subset of chimeric antibodies.

“Humanized” forms of non-human (e.g., murine) antibodies are chimeric antibodies which contain minimal sequence derived from non-human immunoglobulin. For the most part, humanized antibodies are human immunoglobulins (recipient or acceptor antibody) in which hypervariable region residues of the recipient are replaced by hypervariable region residues from a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues which are not found in the recipient antibody or in the donor antibody. These modifications are made to further refine antibody performance such as binding affinity. Generally, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence although the FR regions may include one or more amino acid substitutions that improve binding affinity. The number of these amino acid substitutions in the FR are typically no more than 6 in the H chain, and in the L chain, no more than 3. The humanized antibody optionally also will comprise at least a portion of an immunoglobulin constant region (Fc), typically that of a human immunoglobulin. For further details, see Jones et al., Nature 321:522-525 (1986); Reichmann et al., Nature 332:323-329 (1988); and Presta, Curr. Op. Struct. Biol. 2:593-596 (1992).

“Antibody fragments” comprise a portion of an intact antibody, preferably the antigen binding or variable region of the intact antibody. Examples of antibody fragments include Fab, Fab′, F(ab′)₂, and Fv fragments; diabodies; linear antibodies (see U.S. Pat. No. 5,641,870, Example 2; Zapata et al., Protein Eng. 8(10): 1057-1062[1995]); single-chain antibody molecules; and multispecific antibodies formed from antibody fragments.

Papain digestion of antibodies produces two identical antigen-binding fragments, called “Fab” fragments, and a residual “Fc” fragment, a designation reflecting the ability to crystallize readily. The Fab fragment consists of an entire L chain along with the variable region domain of the H chain (VH), and the first constant domain of one heavy chain (C_{H}1). Each Fab fragment is monovalent with respect to antigen binding, i.e., it has a single antigen-binding site. Pepsin treatment of an antibody yields a single large F(ab′)₂ fragment which roughly corresponds to two disulfide linked Fab fragments having divalent antigen-binding activity and is still capable of cross-linking antigen. Fab′ fragments differ from Fab fragments by having additional few residues at the carboxy terminus of the C_{H}1 domain including one or more cysteines from the antibody hinge region. Fab′-SH is the designation herein for Fab′ in which the cysteine residue(s) of the constant domains bear a free thiol group. F(ab′)₂ antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region, which region is also the part recognized by Fc receptors (FcR) found on certain types of cells.

“Fv” is the minimum antibody fragment which contains a complete antigen-recognition and -binding site. This fragment consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although at a lower affinity than the entire binding site.

“Single-chain Fv” also abbreviated as “sFv” or “scFv” are antibody fragments that comprise the V_{H} and V_{L} antibody domains connected into a single polypeptide chain. Preferably, the sFv polypeptide further comprises a polypeptide linker between the V_{H} and V_{L} domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995, infra.

A “GDM binding agent” is a molecule that binds to a GDM polypeptide (e.g., PN, Prolif, Mes, Pten and DLL3). Example molecules include anti-GDM antibodies, GDM-binding antibody fragments, GDM binding oligopeptides, GDM sense and anti-sense nucleic acid and GDM small molecule antagonists.

A “GDM antagonist” is a molecule that antagonizes (e.g., neutralizes or impedes) the activation or signal transducing ability of a GDM polypeptide, including, for example by blocking the ability of a GDM to transduce a signal, such as by blocking a native ligand from binding or by blocking the GDM from transmitting from a native ligand to a downstream component in a glioma tumor. Example molecules include anti-GDM antibodies, GDM-binding antibody fragments, GDM binding oligopeptides, GDM sense and anti-sense nucleic acid and GDM small molecule antagonists

A “GDM binding oligopeptide” is an oligopeptide that binds, preferably specifically, to a GDM polypeptide, including a receptor, ligand or signaling component, respectively, as described herein. Such oligopeptides may be chemically synthesized using known oligopeptide synthesis methodology or may be prepared and purified using recombinant technology. Such oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more. Such oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and W084/03564; Geysen et al., Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al., J. Immunol. Meth., 102:259-274 (1987); Schoofs et al., J. Immunol., 140:611-616 (1988), Cwirla, S. E. et al. Proc. Natl. Acad. Sci. USA, 87:6378 (1990); Lowman, H. B. et al. Biochemistry, 30:10832 (1991); Clackson, T. et al. Nature, 352: 624 (1991); Marks, J. D. et al., J. Mol. Biol., 222:581 (1991); Kang, A. S. et al. Proc. Natl. Acad. Sci. USA, 88:8363 (1991), and Smith, G. P., Current Opin. Biotechnol., 2:668 (1991).

A “GDM small molecule antagonist” is an organic molecule other than an oligopeptide or antibody as defined herein that inhibits, preferably specifically, a GDM signaling pathway of a GDM polypeptide as described herein. Such GDM signaling pathway inhibition preferably inhibits the growth of glioma tumor cells expressing a GDM polypeptide. Such organic molecules may be identified and chemically synthesized using known methodology (see, e.g., PCT Publication Nos. WO2000/00823 and WO2000/39585). Such organic molecules are usually less than about 2000 daltons in size, alternatively less than about 1500, 750, 500, 250 or 200 daltons in size, are capable of binding, preferably specifically, to a GDM polypeptide as described herein, and may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585).

A GDM antagonist or GDM binding agent (e.g., antibody, polypeptide, oligopeptide or small molecule) “which binds” a target antigen of interest, e.g. a GDM, is one that binds the target with sufficient affinity so as to be a useful diagnostic, prognostic and/or therapeutic agent in targeting a cell or tissue expressing the antigen, and does not significantly cross-react with other proteins. The extent of binding to a non desired marker polypeptide will be less than about 10% of the binding to the particular desired target, as determinable by common techniques such as fluorescence activated cell sorting (FACS) analysis or radioimmunoprecipitation (RIA).

Moreover, the term “specific binding” or “specifically binds to” or is “specific for” a particular GDM polypeptide or an epitope on a particular GDM polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labeled target. In this case, specific binding is indicated if the binding of the labeled target to a probe is competitively inhibited by excess unlabeled target. In one embodiment, such terms refer to binding where a molecule binds to a particular polypeptide or epitope on a particular polypeptide without substantially binding to any other polypeptide or polypeptide epitope. Alternatively, such terms can be described by a molecule having a Kd for the target of at least about 10⁻⁴ M, 10⁻⁵ M, 10⁻⁶ M, 10⁻⁷ M, 10⁻⁸ M, 10⁻⁹ M, 10⁻¹⁰ M, 10^{31 11} M, 10⁻¹² M, or greater.

A GDM antagonist (e.g., PN-, Prolif- or Mes-antagonist) that “inhibits the growth of tumor cells expressing a GDM polypeptide” or a “growth inhibitory” amount of any such molecule is one which results in measurable growth inhibition of cancer cells expressing or overexpressing the appropriate GDM polypeptide. Preferred compositions for use in treatment comprise growth inhibitory amounts of at least one type of GDM antagonist (e.g., anti-GDM antibody, GDM-binding antibody fragment, oligopeptides or small molecule), so as to inhibit growth of glioma tumor cells by greater than 20%, preferably from about 20% to about 50%, and even more preferably, by greater than 50% (e.g., from about 50% to about 100%) as compared to the appropriate control. In one embodiment, growth inhibition can be measured at a molecule concentration of about 0.1 to 30 μg/ml or about 0.5 nM to 200 nM in cell culture, where the growth inhibition is determined 1-10 days after exposure of the tumor cells to the antibody. Growth inhibition of glioma tumor cells in vivo can be determined in various ways such as is described in the Experimental Examples section below. An amount of any of the above molecules of this paragraph is growth inhibitory in vivo if administration of such molecule at about 1 μg/kg to about 100 mg/kg body weight results in reduction in tumor size or tumor cell proliferation within about 5 days to 3 months from the first administration of the antibody, preferably within about 5 to 30 days.

A GDM antagonist which “induces apoptosis” is one which induces programmed cell death of a glioma tumor cell as determined by binding of annexin V, fragmentation of DNA, cell shrinkage, dialation of endoplasmic reticulum, cell fragmentation, and/or formation of membrane vesicles (called apoptotic bodies). The cell is usually one which overexpresses a GDM polypeptide. Various methods are available for evaluating the cellular events associated with apoptosis. For example, phosphatidyl serine (PS) translocation can be measured by annexin binding; DNA fragmentation can be evaluated through DNA laddering; and nuclear/chromatin condensation along with DNA fragmentation can be evaluated by any increase in hypodiploid cells. Preferably, the antibody, oligopeptide or other organic molecule which induces apoptosis is one which results in about 2 to 50 fold, preferably about 5 to 50 fold, and most preferably about 10 to 50 fold, induction of annexin binding relative to untreated cells in an annexin binding assay.

A GDM antagonist which “induces cell death” is one which causes a viable glioma tumor cell to become nonviable. Such glioma tumor cell is one which expresses a GDM polypeptide, preferably overexpresses it, as compared to a non-diseased cell. The GDM polypeptide may be a transmembrane polypeptide expressed on the surface of such cancer cell or may be a polypeptide that is produced and secreted by such a cell. Cell death in vitro may be determined in the absence of complement and immune effector cells to distinguish cell death induced by antibody-dependent cell-mediated cytotoxicity (ADCC) or complement dependent cytotoxicity (CDC). Thus, the assay for cell death may be performed using heat inactivated serum (i.e., in the absence of complement) and in the absence of immune effector cells. The ability to induce cell death can be assessed relative to untreated cells by suitable techniques, such as loss of membrane integrity as evaluated by uptake of propidium iodide (PI), trypan blue (see Moore et al. Cytotechnology 17:1-11 (1995)) or 7AAD. Preferred cell death-inducing GDM antagonists are those which induce PI uptake in the PI uptake assay in BT474 cells.

A “Prolif-antagonist” and an “Mes-antagonist” are GDM antagonists that specifically binds to or otherwise specifically inhibits the activity of a Prolif or Mes GDM marker recited in Table A, respectively. A “PN-antagonist” is a GDM antagonist that specifically binds to or otherwise specifically inhibits the activity of a PN GDM marker recited in Table A, with the exception of DLL3, Nog, Olig1, Olig2, THR, and ASCL1.

An Akt antagonist is any molecule that partially or fully blocks, inhibits, or neutralizes a biological activity of the Akt signaling pathway. Suitable Akt antagonists include antagonist antibodies or antibody fragments, fragments or amino acid sequence variants of native components of the akt signaling pathway (“Akt Polypeptide”), peptides antisense oligonucleotides, small organic molecules, etc. Methods for identifying Akt antagonists may comprise contacting an Akt Polypepide with a candidate molecule and measuring a detectable change in one or more biological activities normally associated with the Akt Polypeptide. Additional example Akt antagonists include: antagonists specifically directed to akt1, akt2, or akt3; antagonists directed at the catalytic or regulatory domain (including the interation with each other) if PIK3 kinase such as PIK3CA, PIK3CB, PIK3CD, PIK3CG, PIK3R1, PIK3R2, PIK3R3, PIK3R4; PDK1; FRAP (e.g., rapamycin); RPS6KB1; SGK; EGFR (e.g., erlotinib TARCEVA®, IGFR. Alternatively, Akt antagonists includes molecules that agonize, stimulate or restore activity of PTEN, INPP5D or INPPL1.

An “anti-mitotic agent” includes a molecule that partially or fully blocks, inhibits or otherwise interferes with mitosis that occurs during cell division. Example of such agents includes: temozolamide, BCNU, CCNU, lomustine, gliadel, etoposide, carmustine, ironotecan, topotecan, procarbazine, cisplatin, carboplatin, cyclophosphamide, vincristine, doxorubicin, dactinomycin, bleomycin, plicamycin, methotrexate, cytarabine, paclitaxel. auristatins, maytansinoids.

An “anti-angiogenic agent” is a molecule that partially or fully blocks, inhibits or otherwise neutralizes the process of angiogenesis or vaculature formation, especially that which is associated with is associated with a disease or disorder. Many angiogenesis antagonists have been identified and are known in the arts, including those listed by Brem, Cancer Control 6(5): 436-458 (1999). Generally, angiogenesis antagonist comprises a molecule targeting a specific angiogenic factor or an angiogenesis pathway. In certain aspects, the angiogenesis antagonist is a protein composition such as an antibody targeting an angiogenic factor. An example angiogenic factor is VEGF (also sometimes known as “VEGF-A”), a 165-amino acid vascular endothelial cell growth factor and related 121-, 189-, and 206-amino acid vascular endothelial cell growth factors, as described by Leung et al. Science, 246:1306 (1989), and Houck et al. Mol. Endocrin., 5:1806 (1991), together with the naturally occurring allelic and processed forms thereof. The term “VEGF” is also used to refer to truncated forms of the polypeptide comprising amino acids 8 to 109 or 1 to 109 of the 165-amino acid human vascular endothelial cell growth factor. Such truncated versions of native VEGF have binding affinity for the Flt-1 (VEGF-R1) and KDR (VEGF-R2) receptors comparable to native VEGF.

An example anti-angioenic factor is a neutralizing anti-VEGF antibody. An “anti-VEGF antibody” is an antibody that binds specifically to VEGF. Preferably, the anti-VEGF antibody of the invention can be used as a therapeutic agent in targeting and interfering with diseases or conditions wherein the VEGF activity is involved. Such anti-VEGF antibody will usually not bind to other VEGF homologues such as VEGF-B or VEGF-C, nor other growth factors such as P1GF, PDGF or bFGF. A preferred anti-VEGF antibody is a monoclonal antibody that binds to the same epitope as the monoclonal anti-VEGF antibody A4.6.1 produced by hybridoma ATCC HB 10709. More preferably the anti-VEGF antibody is a recombinant humanized anti-VEGF monoclonal antibody comprising muated human IgG1 framework regions and antigen-binding complementarity determining regions from the murine anti-hVEGF monoclonal antibody A.4.6.1, and generated according to Presta et al. (1997) Cancer Res. 57:4593-4599 (1997), including but not limited to the antibody known as bevacizumab (BV; Avastin™).

Alternatively, an anti-angiogenic agent can be any small molecule capable of neutralizing, blocking, inhibiting, abrogating, reducing or interfering with VEGF activities including its binding to one or more VEGF receptors (e.g., VEGFR1 and VEGFR2).

A “neural differentiation agent” is a molecule that promotes, causes, stimulates or otherwise induces neuronal precursors (e.g., neural stem cells, transit amplifying cells, neuralblasts, etc.) to differeniate into neurons. Neuronal precursors are cells derived from fetal nervous system, adult brain or the neural crest and are capable of both cell division and the creation of neurons. Neurons are post-mitotic (non-dividing) cells that express proteins involved in axonal projections, action potential propagation, and synaptic transmission. A neuronal differentiation agent is a molecule that induces neuronal precursors to decrease their rate of proliferation and increase their expression of proteins involved in axonal outgrowth, action potential generation, and synaptic transmission. Example markers of neuronal differentiation include, but are not limited to MAP2, beta-tubulin, GAD65 and GAP43. Example neural differentiation agents include, but are not limited to: retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-4, jagged 1 antagonist, jagged 2 antagonist; numb agonist or numb-like agonist.

Antibody “effector functions” refer to those biological activities attributable to the Fc region (a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation. “Antibody-dependent cell-mediated cytotoxicity” or “ADCC” refers to a form of cytotoxicity in which secreted Ig bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. The antibodies “arm” the cytotoxic cells and are absolutely required for such killing. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-92 (1991). To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. (USA) 95:652-656 (1998).

“Fc receptor” or “FcR” describes a receptor that binds to the Fc region of an antibody. The preferred FcR is a native sequence human FcR. Moreover, a preferred FcR is one which binds an IgG antibody (a gamma receptor) and includes receptors of the FcγRI, FcγRII and FcγRIII subclasses, including allelic variants and alternatively spliced forms of these receptors. FcγRII receptors include FcγRIIA (an “activating receptor”) and FcγRIIB (an “inhibiting receptor”), which have similar amino acid sequences that differ primarily in the cytoplasmic domains thereof. Activating receptor FcγRIIA contains an immunoreceptor tyrosine-based activation motif (ITAM) in its cytoplasmic domain. Inhibiting receptor FcγRIIB contains an immunoreceptor tyrosine-based inhibition motif (ITIM) in its cytoplasmic domain. (see review M. in Daëron, Annu. Rev. Immunol. 15:203-234 (1997)). FcRs are reviewed in Ravetch and Kinet, Annu. Rev. Immunol. 9:457-492 (1991); Capel et al., Immunomethods 4:25-34 (1994); and de Haas et al., J. Lab. Clin. Med. 126:330-41 (1995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 117:587 (1976) and Kim et al., J. Immunol. 24:249 (1994)).

“Human effector cells” are leukocytes which express one or more FcRs and perform effector functions. Preferably, the cells express at least FcγRIII and perform ADCC effector function. Examples of human leukocytes which mediate ADCC include peripheral blood mononuclear cells (PBMC), natural killer (NK) cells, monocytes, cytotoxic T cells and neutrophils; with PBMCs and NK cells being preferred. The effector cells may be isolated from a native source, e.g., from blood.

“Complement dependent cytotoxicity” or “CDC” refers to the lysis of a target cell in the presence of complement. Activation of the classical complement pathway is initiated by the binding of the first component of the complement system (C1q) to antibodies (of the appropriate subclass) which are bound to their cognate antigen. To assess complement activation, a CDC assay, e.g., as described in Gazzano-Santoro et al., J. Immunol. Methods 202:163 (1996), may be performed.

A “GDM-expressing glioma” optionally produces sufficient levels of GDM polypeptide on the surface of cells thereof, such that a GDM polypeptide antagonist can bind thereto or a GDM small molecule antagonist can otherwise target and have a therapeutic effect with respect to the glioma.

In another embodiment, a “GDM-expressing glioma” optionally produces and secretes sufficient levels of GDM polypeptide, such that a GDM polypeptide antagonist can bind thereto or a GDM small molecule antagonist can otherwise target and have a therapeutic effect with respect to the cancer. With respect to antagonists, such molecules may be an antisense oligonucleotide which reduces, inhibits or prevents production and secretion of the secreted GDM polypeptide by tumor cells.

A glioma tumor that “overexpresses” a GDM polypeptide is one which has significantly higher levels of GDM at the cell surface thereof, or that produces and secretes, compared to a noncancerous cell of the same tissue type. Such overexpression may result from gene amplification or by increased transcription or translation. Various diagnostic or prognostic assays that measure enhanced expression of GDM resulting in increased levels at the cell surface or that which is secreted, such as immunohistochemistry assay using anti-GDM antibodies, FACS analysis, etc. Alternatively, the levels of GDM polypeptide-encoding nucleic acid or mRNA can be measured in the cell, e.g., via fluorescent in situ hybridization using a nucleic acid based probe corresponding to a GDM-encoding nucleic acid or the complement thereof; (FISH; see WO98/45479 published October, 1998), Southern blotting, Northern blotting, or polymerase chain reaction (PCR) techniques, such as real time quantitative PCR (RT-PCR). Alternatively, GDM polypeptide overexpression is determinable by measuring shed antigen in a biological fluid such as serum, e.g, using antibody-based assays (see also, e.g., U.S. Pat. No. 4,933,294 issued Jun. 12, 1990; WO91/05264 published Apr. 18, 1991; U.S. Pat. 5,401,638 issued Mar. 28, 1995; and Sias et al., J. Immunol. Methods 132:73-80 (1990)). In addition to the above assays, various in vivo assays are available to the skilled practitioner. For example, one may expose cells within the body of the patient to an antibody which is optionally labeled with a detectable label, e.g., a radioactive isotope, and binding of the antibody to cells in the patient can be evaluated, e.g., by external scanning for radioactivity or by analyzing a biopsy taken from a patient previously exposed to the therapeutic agent.

As used herein, the term “immunoadhesin” designates antibody-like molecules which combine the binding specificity of a heterologous protein (an “adhesin”) with the effector functions of immunoglobulin constant domains. Structurally, the immunoadhesins comprise a fusion of an amino acid sequence with the desired binding specificity which is other than the antigen recognition and binding site of an antibody (i. e., is “heterologous”), and an immunoglobulin constant domain sequence. The adhesin part of an immunoadhesin molecule typically is a contiguous amino acid sequence comprising at least the binding site of a receptor or a ligand. The immunoglobulin constant domain sequence in the immunoadhesin may be obtained from any immunoglobulin, such as IgG-1, IgG-2, IgG-3, or IgG-4 subtypes, IgA (including IgA-1 and IgA-2), IgE, IgD or IgM.

The word “label” when used herein refers to a detectable compound or composition which is conjugated directly or indirectly to the antibody, oligopeptide or other organic molecule so as to generate a “labeled” antibody, oligopeptide or other organic molecule. The label may be detectable by itself (e.g. radioisotope labels or fluorescent labels) or, in the case of an enzymatic label, may catalyze chemical alteration of a substrate compound or composition which is detectable.

The term “cytotoxic agent” as used herein refers to a substance that inhibits or prevents the function of cells and/or causes destruction of cells. The term is intended to include radioactive isotopes (e.g., At²¹¹, I¹³¹, I¹²⁵, Y⁹⁰, Re¹⁸⁶, Re¹⁸⁸, Sm¹⁵³, Bi²¹², p³² and radioactive isotopes of Lu), chemotherapeutic agents, enzymes and fragments thereof such as nucleolytic enzymes, antibiotics, and toxins such as small molecule toxins or enzymatically active toxins of bacterial, fungal, plant or animal origin, including fragments and/or variants thereof, and the various antitumor or anticancer agents disclosed below. Other cytotoxic agents are described below. A tumoricidal agent causes destruction of tumor cells.

A “chemotherapeutic agent” is a chemical compound useful in the treatment of cancer. Examples of chemotherapeutic agents include hydroxyureataxanes (such as paclitaxel and doxetaxel) and/or anthracycline antibiotics; alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegaI1(see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, triemetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfomithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANETM Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylomithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATINTM) combined with 5-FU and leucovovin.

Also included in this definition are anti-hormonal agents that act to regulate, reduce, block, or inhibit the effects of hormones that can promote the growth of cancer, and are often in the form of systemic, or whole-body treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in abherant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

A “growth inhibitory agent” when used herein refers to a compound or composition which inhibits growth of a cell, especially a GDM-expressing glioma cell, either in vitro or in vivo. Thus, the growth inhibitory agent may be one which significantly reduces the percentage of GDM-expressing cells in S phase. Examples of growth inhibitory agents include agents that block cell cycle progression (at a place other than S phase), such as agents that induce G1 arrest and M-phase arrest. Classical M-phase blockers include the vincas (vincristine and vinblastine), taxanes, and topoisomerase II inhibitors such as doxorubicin, epirubicin, daunorubicin, etoposide, and bleomycin. Those agents that arrest G1 also spill over into S-phase arrest, for example, DNA alkylating agents such as tamoxifen, prednisone, dacarbazine, mechlorethamine, cisplatin, methotrexate, 5-fluorouracil, and ara-C. Further information can be found in The Molecular Basis of Cancer, Mendelsohn and Israel, eds., Chapter 1, entitled “Cell cycle regulation, oncogenes, and antineoplastic drugs” by Murakami et al. (WB Saunders: Philadelphia, 1995), especially p. 13. The taxanes or hydroxyureataxanes (paclitaxel and docetaxel) are anticancer drugs both derived from the yew tree. Docetaxel (TAXOTERE®, Rhone-Poulenc Rorer), derived from the European yew, is a semisynthetic analogue of paclitaxel (TAXOL®, Bristol-Myers Squibb). These molecules promote the assembly of microtubules from tubulin dimers and stabilize microtubules by preventing depolymerization, which results in the inhibition of mitosis in cells.

“Doxorubicin” is an anthracycline antibiotic. The full chemical name of doxorubicin is (8S-cis)-10-[(3-amino-2,3,6-trideoxy-α-L-lyxo-hexapyranosyl)oxy]-7,8,9,10-tetrahydro-6, 8,11-trihydroxy-8-(hydroxyacetyl)-1-methoxy-5,12-naphthacenedione.

The term “cytokine” is a generic term for proteins released by one cell population which act on another cell as intercellular mediators. Examples of such cytokines are lymphokines, monokines, and traditional polypeptide hormones. Included among the cytokines are growth hormone such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor; integrin; thrombopoietin (TPO); nerve growth factors such as NGF-β; platelet-growth factor; transforming growth factors (TGFs) such as TGF-α and TGF-β; insulin-like growth factor-I and -II; erythropoietin (EPO); osteoinductive factors; interferons such as interferon -α, -β, and -γ; colony stimulating factors (CSFs) such as macrophage-CSF (M-CSF); granulocyte-macrophage-CSF (GM-CSF); and granulocyte-CSF (G-CSF); interleukins (ILs) such as IL-1, IL-1a, IL-2, IL-3, IL-4, IL-5, IL-6, IL-7, IL-8, IL-9, IL-11, IL-12; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

The term “package insert” is used to refer to instructions customarily included in commercial packages of therapeutic products, that contain information about the indications, usage, dosage, administration, contraindications and/or warnings concerning the use of such therapeutic products.

The term “hierarchical clustering” means a method of grouping sets of samples based on their similarity of gene expression. The standard algorithm used recursively computes a dendrogram that assembles all the elements into a tree, starting from a correlation matrix. Eisen, M.B. etal., P.N.A.S. 95:14863-14868 (1998).

The term “k means clustering” means is a method of grouping sets of samples based on their similarity of gene expression. In k-means clustering, all samples are initially assigned at random to one of a number of clusters. The representative or mean values of gene expression in each sample cluster are then computed and each sample is reassigned to the cluster to which it shows the closest similarity. This procudere is reiterated until a stable structure is achieved. Duda, R. O. and Hart, P. E., Pattern Classification and Scene Analysis, Wiley, N.Y. (1973).

The term “voting scheme” means is a method of assigning tumors to groups based on comparing the number of GDMs for each tumor subtype that are expressed at or above a certain expression level. Freije et al., supra.

TABLE 2

| | | |
|---|---|---|
| Reference | XXXXXXXXXXXXXXX | (Length = 15 amino acids) |
| Comparison | XXXXXYYYYYYY | (Length = 12 amino acids) |
| Protein | | |

% amino acid sequence identity = (the number of identically matching amino acid residues between the two polypeptide sequences as determined by ALIGN-2) divided by (the total number of amino acid residues of the reference polypeptide) = 5 divided by 15 = 33.3%

TABLE 3

| | | |
|---|---|---|
| Reference | XXXXXXXXXX | (Length = 10 amino acids) |
| Comparison | XXXXXYYYYYYZZYZ | (Length = 15 amino acids) |
| Protein | | |

% amino acid sequence identity = (the number of identically matching amino acid residues between the two polypeptide sequences as determined by ALIGN-2) divided by (the total number of amino acid residues of the reference polypeptide) = 5 divided by 10 = 50%

TABLE 4

| | | |
|---|---|---|
| Reference- | NNNNNNNNNNNNNN | (Length = 14 nucleotides) |
| DNA | | |
| Comparison | NNNNNNLLLLLLLLLL | (Length = 16 nucleotides) |
| DNA | | |

% nucleic acid sequence identity = (the number of identically matching nucleotides between the two nucleic acid sequences as determined by ALIGN-2) divided by (the total number of nucleotides of the reference-DNA nucleic acid sequence) = 6 divided by 14 = 42.9%

TABLE 5

| | | |
|---|---|---|
| Reference-DNA | NNNNNNNNNNNN | (Length = 12 nucleotides) |
| Comparison DNA | NNNNLLLVV | (Length = 9 nucleotides) |

% nucleic acid sequence identity = (the number of identically matching nucleotides between the two nucleic acid sequences as determined by ALIGN-2) divided by (the total number of nucleotides of the reference-DNA nucleic acid sequence) = 4 divided by 12 = 33.3%

II. Compositions and Methods of the Invention

A. Methods and Meanings

At present, high-grade gliomas are diagnosed by histopathological criteria and known robust prognostic factors for most of these tumors are limited to tumor grade and patient age. The widespread acceptance that losses on chrs 1p and 19q are of prognostic value in oligodendroglioma (Cairncross et al., J. Natl Cancer Inst. 90: 1473-1479 (1998) has spurred interest in developing molecular markers to predict outcome and response to treatment across a broader population of gliomas. While numerous genetic alterations have been described in GBM, and some, such as EGFR amplification and p53 mutation, appear to distinguish between primary and secondary GBMs (von Deimling et al., Glia 15: 328-338 (1995); Watanabe et al., Brain Pathol. 6: 217-223 (1996), such markers are of marginal utility in predicting outcome or guiding decisions about disease management. Importantly, recent expression profiling studies have revealed that molecular classification of gliomas can be of prognostic value (Freije et al., Cancer Res. 64: 6503-6510 (2004); Nutt et al., Cancer Res. 63: 1602-1607 (2003). In the current study, we identify molecular alterations associated with tumor aggressiveness as well as with disease progression and provide evidence to suggest that molecular classification might be used to predict responses to targeted therapies.

Tumor Subclasses have Prognostic Value and Delineate a Pattern of Disease Progression

Applicants describes herein, a novel prognostic classification scheme for high-grade astrocytoma that assigns tumors to subtypes based on similarity to defined expression signatures. Each of the three molecular subtypes of glioma resembles a distinct set of tissues and is enriched for markers of different aspects of tissue growth. While the current analysis utilizes a set of 35 signature genes, these markers are representative of much longer lists of markers that can be used to identify each tumor subtype. One tumor subtype, which we term proneural (PN), is distinguished by markedly better prognosis and expresses genes associated with normal brain and the process of neurogenesis. Two poor prognosis subtypes that are characterized by a resemblance to either highly proliferative cell lines or tissues of mesenchymal origin show activation of gene expression programs indicative of cell proliferation or angiogenesis, respectively. We speculate that the poor survival associated with the Mes and Prolif tumor types is related to a growth advantage conferred by either a rapid rate of cell division or enhanced survival of tumor cells afforded by neovascularization. While the mere presence or absence or neovascularization or mitotic figures did not distinguish subclasses, this is to be expected, sine these features are hallmarks of nearly all glioblastoma multiforme. Previous studies have suggested the prognostic value of markers of proliferation or angiogenesis in glioma (Ho et al., Am. J. Clin. Pathol. 119: 715-722 (2003); Hsu et al., Cancer Res. 56: 5684-5691 (1996); Osada et al., Anticancer Res. 24: 547-552 (2004), but have not indicated the existence of distinct tumor subsets that are differentially associated with these processes. Of note, our Prolif and Mes glioma subtypes are characterized by expression of subsets of markers of a wound-healing signature that has been associated with poor outcome in several epithelial tumor types. See Table A; (Chang et al., P.N.A.S. (USA) 85: 704-710 (2005).

The tumor subtypes identified in the current study bare resemblance to previously-reported prognostic subtypes identified by expression profiling. In particular, three previously-published studies report phenotypes that closely resemble the PN and Mes tumor subtypes that we describe (Freije et al., supra; Liang et al., P.N.A.S. (USA) 102: 5814-5819 (2005); Nigro et al., Cancer Res. 65: 1678-1686 (2005). In addition, previous work (Godard et al., Cancer Res. 63: 6613-6625 (2003) highlights a cluster of angiogenic genes defining a tumor subpopulation that appears similar to our Mes tumor subtype. Our observation of a mutually-exclusive pattern of expression of PN vs. Mes markers helps explain the consensus regarding the existence of these two tumor subtypes. Even within tumor specimens that express both PN and Mes markers, we find a non-overlapping spatial distribution of expression. The strong association between LOH10 and the distinction between the Mes and PN signatures is consistent with previous findings linking LOH10 to prognostic expression signatures (Nigro et al., Cancer Res. 65: 1678-1686 (2005) and the association between an angiogenic phenotype and LOH10 is consistent with the demonstration of anti-angiogenic actions of chr 10 introduction into GBM cell lines (Hsu et al., Cancer Res. 56: 5684-5691 (1996).

The existence of a distinct tumor subtype that is enriched for markers of proliferation has been noted by one previous report (Freije et al., supra.), but not described in other studies. Our findings indicate that the Prolif signature is less exclusive than PN or Mes signatures and that the proportion of gliomas that occur with a Prolif signature varies across sample populations obtained from different institutions. In support of our categorization of the Prolif tumor subclass as a distinct molecular subtype of tumor, we point to the existence in Prolif tumors of a pattern of genomic alterations that distinguishes this tumor subtype. Most notably, gains of the PIK3R3 locus on chr1 appear to be a feature that is unique to tumors of the Prolif class. The existence of a genomic alteration unique to tumors bearing the Prolif signature argues in favor of the designation of these tumors as a distinct subclass and suggests that epidemiological factors may have influenced the incidence of this subclass in the investigated populations.

The striking mutual exclusivity of the PN and Mes tumor signatures suggests the possibility that these tumor subtypes reflect distinct disease entities, perhaps arising from different cell types of origin. By studying matched pairs of primary and recurrent tumors from the same patients, however, we observe that some tumors that originally arise as PN or Prolif subtype recur with a Mes signature. Focal expression of CHI3L1/YKL40, a marker of the Mes phenotype, is seen in primary tumors, including those that shift to Mes class upon recurrence. Notably, no instances are seen of tumors gaining appreciable PN character between initial presentation and recurrence. Taken together with the ability of neural stem cell lines to shift from PN to Mes signature, the ability of tumors to change subclass suggests that the tumor subtypes may represent alternate differentiation states of disease. We cannot, however, rule out the possibility that some apparent shifts may reflect tumor heterogeneity rather than temporal changes in tumor character. In addition, our experimental design does not allow us to distinguish between alterations in gene expression that reflect disease progression from those which are elicited by treatment effects. Nevertheless, our finding of unidirectional tumor subclass shifts suggest the possibility that tumor cells can acquire the Mes phenotype as a result of accumulation of genetic or epigenetic abnormalities. The older age of patients with Mes subtype tumors is consistent with such a hypothesis.

While we have no direct evidence for molecular events which underlie the apparent shift in tumor cell signature, the strong correlation between losses of chr 10 and the Mes signature may offer an important insight into the biology of disease progression. Regardless of underlying mechanism, shifts towards the Mes phenotype appear to be a common pattern of disease progression and are reminiscent of epithelial-to-mesenchymal transitions (EMT) that are associated with increased malignant behavior of epithelial tumors. Consistent with the central role of Akt activation in EMT [Larue and Bellacosa, Oncogene 24: 7443 (2005)], our data suggest that Akt plays a role in inducing a mesenchymal transition in gliomas.

Markers in Notch and Akt Signaling Predict Glioma Aggressiveness

Our findings demonstrate evidence at genomic, mRNA and protein levels for activating alterations of Akt signaling in tumors of poor prognosis subtypes. A wealth of previous data supports a role for akt signaling in promoting the formation and growth of high-grade glial malignancies (Knobbe et al., Neuro-Oncology 4: 196-211 (2002); Sonoda et al., Cancer Res. 61: 6674-6678 (2001). A series of elegant studies in genetically-engineered mouse models has convincingly demonstrated a role for akt in promoting formation and growth of glial malignancies (Holland et al., Nature Genetics 25: 55-57 (2000); Rajasekhar et al., Mol. Cell 12: 889-901 (2003); Uhrbom et al., Cancer Res. 62: 5551-5558 (2002); Xiao et al., Cancer Res. 65: 5172-5180 (2005). In human tumors, both EGFR amplification and PTEN deletion are well-known alterations that activate akt and are specifically associated with the distinction between GBM vs. lower grade lesions (Stiles et al., Mol. Cell Biol. 22: 3842-3851 (2002). More recently, both mutations in PIK3CA and balanced copy number increases in PIK3CA and PIK3CD have been described in AA and GBM (Broderick et al., Cancer Res. 64: 5048-5050 (2004); Mizoguchi et al., Brain Pathol 14: 372-377 (2004); Samuels et al., Science 304: 54 (2004). While the prognostic value of EGFR amplification or genetic changes in PI3K subunits is not clear, several studies have shown that losses on chr 10, loss of the PTEN locus, or enhanced PI3K signaling are all associated with poor outcome in GBM (Chakravarti et al., J. Clin. Oncol. 22: 1926-1933 (2004); Lin et al., Clin. Cancer Res. 4: 2447-2454 (1998); Schmidt et al., J. Neur. Exp. Neurol. 61: 321-328 (2002); Smith et al., J. Nat. Cancer Inst. 93: 1246-1256 (2001); Tada et al., J. Neurosurg. 95: 651-659 (2001). Activation of PI3K/akt signaling is implicated in several biological processes which confer a growth advantage, including proliferation, survival., and angiogenesis (Abe et al., Cancer Res. 63: 2300-2305 (2003); Pore et al., Cancer Res. 63: 236-241 (2003); Su et al., Cancer Res. 63: 3585-3592 (2003). Thus, we speculate that the poor outcome of Prolif and Mes subtype tumors results from actions of PI3K/akt signaling to promote more aggressive growth patterns characterized by high rate of proliferation or neoangiogenesis, respectively. Current findings do not lend a clear hypothesis to explain the divergence between the proliferative vs. angiogenic manifestations of akt signaling in the two poor prognosis subtypes, but one possibility is that more frequent loss of loci on chr 10p or gains on chr 7 in Mes tumors may contribute to this distinction. The high frequency of markers encoded on ch19 is interesting in this regard.

Activation of Notch1 signaling has been recently linked to several malignancies, including glioma (Fan et al., Cancer Res. 64: 7787-7793 (2004); Purow et al., Cancer Res. 65: 2353-2363 (2005); Radtke and Clevers, Science 307: 1904-1909 (2005); Weng et al., Science 306: 269-271 (2004). Our observations demonstrate prognostic value of Notch pathway markers in high-grade gliomas. Specifically, we find that Notch1 nuclear staining and mRNA for several Notch pathway elements are significantly enriched in the better outcome PN tumor subtype as compared to poor prognosis subtypes. Further, we find in two independent sample populations that expression of mRNA for DLL3 is correlated with longer survival, particularly in cases where PTEN expression is high. While several interpretations are possible, one interesting possibility is that in the presence of intact PTEN, inhibitory activity of DLL3 on notch signaling [Ladi et al., J. Cell Biol. 170: 983-992 (2005)] may limit tumor growth by promoting a more differentiated phenotype. Regardless of the precise role of Notch signaling, the prognostic value of our two-gene PTEN & DLL3 Cox model clearly points to akt and Notch signaling as major determinants of tumor growth.

Parallels Between Regulation of Glioma Growth and Forebrain Neurogenesis.

The current investigation links prognostic tumor subtypes to differences in relative expression of neural stem cell vs. neuroblast markers as well as to differences in Akt and Notch signaling elements. One model for human gliomas is that all molecularly-defined subtypes arise from similar cell type(s)-of-origin, but some tumors are maintained in more undifferentiated neural stem cell-like (Mes) or transit-amplying-like (Prolif) phenotypes, while others (PN) adopt a phenotype closer to that of neuroblasts or immature neurons. This model, supported by animal studies [Bachoo et al., Cancer Cell 1: 269-277 (2002); Fomchenko and Holland, Exp. Cell Res. 306: 323-329 (2005)], makes no specific prediction as to what stage(s) along the differentiation axis from neural stem cell to neuronal or glial lineage(s) the cell type(s)-of-origin for high-grade gliomas reside and slows that the phenotype of the tumor may be dictated by molecular alterations in signaling pathways. In light of the critical roles that PTEN and notch expert suring forebrain neurogenesis to maintain neural stem cells or progenitors in a proliferating undifferentiated state (Groszer et al., Science 294: 2186-2189 (2001); Sakamoto et al., J. Biol. Chem. 278: 44808-44815 (2003); Yoon and Gaiano, Nature Neurosci. 8: 709-715 (2005), our findings suggest that aggressiveness of glioma growth may be largely governed by processes that regulate cell fate choices during neurogenesis.

The phenotype of the newly-defined tumor subtypes parallels stages in neurogenesis in the adult forebrain. Similar to committed neuronal (or neuronal-oligodendroglial) precursors, tumors of the PN subtype appear to have a low rate of proliferation, and express markers associated with neuroblasts and immature neurons the transcription factors OLIG2 and Asc11 along with other neuronal lineage markers. In contrast, Mes and Prolif subtype tumors lack markers of neuronal lineages, but recapitulate aspects of neural stem cells and/or transit amplifying cells. The parallel between the apparently rapid rate of proliferation of Prolif tumors and the transit amplifying cells is readily apparent. In addition, we find that some tumors of the Prolif subtype, but not other classes, are characterized by robust expression of MELK, a marker of rapidly proliferating multipotential precursor cells in the rodent forebrain. Nakano et al., J. Cell Biol. 170: 413 (2005). Further, the EGFR amplifications in tumors of both Prolif and Mes subclasses parallels the responsiveness of both neural stem cells and transit amplifying cells to EGF (Doetsch et al., Neuron 36: 1021-1034 (2002). The expression of smooth muscle, endothelial cells, and cartilage markers by Mes tumors is reminiscent of the reported multipotentiality of neural stem cells from adult forebrain. Bani-Yaghoub etal., Development 131: 4287-4298 (2004); Rietze et al., Nature 412: 736-739 (2001); Sieber-Blum, Developmental Neuroscience 25:273-278 (2003); Wurmser et al., Nature 430: 350-356 (2004). One caveat in interpretation, however, pertains to the possibility that tumor expression profiles may be confounded by recruitment of stem cell-like populations to the tumor mass.

Intriguingly, the parallels between the Mes tumor phenotype and neural stem cells extend include a recapitulation of the close association seen between neural stem cells and endothelial cells. In contrast to other tumor subtypes, Mes tumors display robust expression of VEGF, its receptors, and markers of endothelial cells. Recent findings indicate that VEGF promotes proliferation and survival of adult forebrain neural stem cells and demonstrate that secreted factors from endothelial cells also promote neural stem cell proliferation (Cao et al., Nature Genetics 36: 827-835 (2004); Fabel et al., Eur. J. Neurosci. 18: 2803-2812 (2003); Jin et al., P.N.A.S. (USA) 99: 11946-11950 (2002); Maurer et al., Neurosci. Lett. 344: 165-168 (2003); Schanzer et al., Brain Pathol. 14: 237-248 (2004); Shen et al., Science 304: 1338-1340 (2004); Yasuhara et al., Reviews Neurosci. 15: 293-307 (2004); Zhu et al., FASEB J. 17: 186-193 (2003). It is interesting to speculate that the growth of tumor cells of the Mes tumor phenotype may be supported by the actions of increased levels of VEGF and/or endothelial-derived factors. In this regard, therapies that target VEGF or its receptors might prove beneficial in not only targeting neovasculature, but also directly inhibiting growth of tumor cells which manifest a neural stem cell-like biology. Targeted inactivation of VEGF in the neural tube has been recently demonstrated to produce both vascular defects and a profound degree of neuronal apoptosis in the murine forebrain (Raab et al., Thrombosis Haemostasis 91: 595-605 (2004).

Therapeutic Implications

The present findings offer several implications for the development of effective therapies for glioma. First, the current investigation adds to the growing consensus that that optimal treatment of glial malignancies may rely on treatment regimens targeted at distinct molecular categories of tumor (Mischel et al., Cancer Biol. Therapy 2: 242-247 (2003); Newton, Expert Rev. Antican. Ther. 4: 105-128 (2004); Rao et al., Frontier Biosci. 8: e270-280 (2003). Our in vitro findings suggest that the molecular signatures we define can predict responses to agents targeting specific signaling pathways. Second, our findings support the value of targeting both Akt and Notch pathways in the development of novel therapeutic regimens for high-grade glioma. Third, the suggestion that tumor recurrence after standard therapies may be accompanied by a phenotypic shift into a mesenchymal, angiogenic state underscores the value of targeting this aggressive phenotypic state even in tumors with a less aggressive phenotype. Finally, correlations between stem cell biology and more aggressive glioma phenotypes suggest that greater understanding of forebrain neurogenesis may lead to novel insights for therapeutic intervention in glial malignancies.

A. Anti-GDM Antibodies

In one embodiment, the present invention provides the use of anti-GDM antibodies, which may find use herein as therapeutic, diagnostic and/or prognostic agents in determining the severity of and/or prognosing the disease course of glioma. Exemplary antibodies that may be used for such purposes include polyclonal, monoclonal, humanized, bispecific, and heteroconjugate antibodies.

1. Polyclonal Antibodies

Polyclonal antibodies are preferably raised in animals by multiple subcutaneous (sc) or intraperitoneal (ip) injections of the relevant antigen and an adjuvant. It may be useful to conjugate the relevant antigen (especially when synthetic peptides are used) to a protein that is immunogenic in the species to be immunized. For example, the antigen can be conjugated to keyhole limpet hemocyanin (KLH), serum albumin, bovine thyroglobulin, or soybean trypsin inhibitor, using a bifunctional or derivatizing agent, e.g., maleimidobenzoyl sulfosuccinimide ester (conjugation through cysteine residues), N-hydroxysuccinimide (through lysine residues), glutaraldehyde, succinic anhydride, SOCl₂, or R¹N═C═NR, where R and R¹ are different alkyl groups.

Animals are immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later, the animals are boosted with ⅕ to 1/10 the original amount of peptide or conjugate in Freund's complete adjuvant by subcutaneous injection at multiple sites. Seven to 14 days later, the animals are bled and the serum is assayed for antibody titer. Animals are boosted until the titer plateaus. Conjugates also can be made in recombinant cell culture as protein fusions. Also, aggregating agents such as alum are suitably used to enhance the immune response.

2. Monoclonal Antibodies

Monoclonal antibodies may be made using the hybridoma method first described by Kohler et al., Nature, 256:495 (1975), or may be made by recombinant DNA methods (U.S. Pat. No. 4,816,567).

In the hybridoma method, a mouse or other appropriate host animal, such as a hamster, is immunized as described above to elicit lymphocytes that produce or are capable of producing antibodies that will specifically bind to the protein used for immunization. Alternatively, lymphocytes may be immunized in vitro. After immunization, lymphocytes are isolated and then fused with a myeloma cell line using a suitable fusing agent, such as polyethylene glycol, to form a hybridoma cell (Goding, Monoclonal Antibodies: Principles and Practice, pp. 59-103 (Academic Press, 1986)).

The hybridoma cells thus prepared are seeded and grown in a suitable culture medium which medium preferably contains one or more substances that inhibit the growth or survival of the unfused, parental myeloma cells (also referred to as fusion partner). For example, if the parental myeloma cells lack the enzyme hypoxanthine guanine phosphoribosyl transferase (HGPRT or HPRT), the selective culture medium for the hybridomas typically will include hypoxanthine, aminopterin, and thymidine (HAT medium), which substances prevent the growth of HGPRT-deficient cells.

Preferred fusion partner myeloma cells are those that fuse efficiently, support stable high-level production of antibody by the selected antibody-producing cells, and are sensitive to a selective medium that selects against the unfused parental cells. Preferred myeloma cell lines are murine myeloma lines, such as those derived from MOPC-21 and MPC-11 mouse tumors available from the Salk Institute Cell Distribution Center, San Diego, Calif. USA, and SP-2 and derivatives e.g., X63-Ag8-653 cells available from the American Type Culture Collection, Manassas, Va., USA. Human myeloma and mouse-human heteromyeloma cell lines also have been described for the production of human monoclonal antibodies (Kozbor, J. Immunol., 133:3001 (1984); and Brodeur et al., Monoclonal Antibody Production Techniques and Applications, pp. 51-63 (Marcel Dekker, Inc., New York, 1987)).

Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunosorbent assay (ELISA).

The binding affinity of the monoclonal antibody can, for example, be determined by the Scatchard analysis described in Munson et al., Anal. Biochem., 107:220 (1980).

Once hybridoma cells that produce antibodies of the desired specificity, affinity, and/or activity are identified, the clones may be subcloned by limiting dilution procedures and grown by standard methods (Goding, Monoclonal Antibodies: Principles and Practice, pp.59-103 (Academic Press, 1986)). Suitable culture media for this purpose include, for example, D-MEM or RPMI-1640 medium. In addition, the hybridoma cells may be grown in vivo as ascites tumors in an animal e.g, by i.p. injection of the cells into mice.

The monoclonal antibodies secreted by the subclones are suitably separated from the culture medium, ascites fluid, or serum by conventional antibody purification procedures such as, for example, affinity chromatography (e.g., using protein A or protein G-Sepharose) or ion-exchange chromatography, hydroxylapatite chromatography, gel electrophoresis, dialysis, etc.

DNA encoding the monoclonal antibodies is readily isolated and sequenced using conventional procedures (e.g., by using oligonucleotide probes that are capable of binding specifically to genes encoding the heavy and light chains of murine antibodies). The hybridoma cells serve as a preferred source of such DNA. Once isolated, the DNA may be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Review articles on recombinant expression in bacteria of DNA encoding the antibody include Skerra et al., Curr. Opinion in Immunol., 5:256-262 (1993) and Püickthun, Immunol. Revs. 130:151-188 (1992).

In a further embodiment, monoclonal antibodies or antibody fragments can be isolated from antibody phage libraries generated using the techniques described in McCafferty et al., Nature, 348:552-554 (1990). Clackson et al., Nature, 352:624-628 (1991) and Marks et al., J. Mol. Biol., 222:581-597 (1991) describe the isolation of murine and human antibodies, respectively, using phage libraries. Subsequent publications describe the production of high affinity (nM range) human antibodies by chain shuffling (Marks et al., Bio/Technology, 10:779-783 (1992)), as well as combinatorial infection and in vivo recombination as a strategy for constructing very large phage libraries (Waterhouse et al., Nuc. Acids. Res. 21:2265-2266 (1993)). Thus, these techniques are viable alternatives to traditional monoclonal antibody hybridoma techniques for isolation of monoclonal antibodies.

The DNA that encodes the antibody may be modified to produce chimeric or fusion antibody polypeptides, for example, by substituting human heavy chain and light chain constant domain (C_{H} and C_{L}) sequences for the homologous murine sequences (U.S. Pat. No. 4,816,567; and Morrison, et al., Proc. Natl Acad. Sci. USA, 81:6851 (1984)), or by fusing the immunoglobulin coding sequence with all or part of the coding sequence for a non-immunoglobulin polypeptide (heterologous polypeptide). The non-immunoglobulin polypeptide sequences can substitute for the constant domains of an antibody, or they are substituted for the variable domains of one antigen-combining site of an antibody to create a chimeric bivalent antibody comprising one antigen-combining site having specificity for an antigen and another antigen-combining site having specificity for a different antigen.

3. Human and Humanized Antibodies

The anti-GDM antibodies useful in the practice of the invention may further comprise humanized antibodies or human antibodies. Humanized forms of non-human (e.g., murine) antibodies are chimeric immunoglobulins, immunoglobulin chains or fragments thereof (such as Fv, Fab, Fab′, F(ab′)₂ or other antigen-binding subsequences of antibodies) which contain minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as “import” residues, which are typically taken from an “import” variable domain. Humanization can be essentially performed following the method of Winter and co-workers [Jones et al., Nature, 321:522-525 (1986); Riechmann et al., Nature, 332:323-327 (1988); Verhoeyen et al., Science, 239:1534-1536 (1988)], by substituting rodent CDRs or CDR sequences for the corresponding sequences of a human antibody. Accordingly, such “humanized” antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

The choice of human variable domains, both light and heavy, to be used in making the humanized antibodies is very important to reduce antigenicity and HAMA response (human anti-mouse antibody) when the antibody is intended for human therapeutic use. According to the so-called “best-fit” method, the sequence of the variable domain of a rodent antibody is screened against the entire library of known human variable domain sequences. The human V domain sequence which is closest to that of the rodent is identified and the human framework region (FR) within it accepted for the humanized antibody (Sims et al., J. Immunol. 151:2296 (1993); Chothia et al., J. Mol. Biol., 196:901 (1987)). Another method uses a particular framework region derived from the consensus sequence of all human antibodies of a particular subgroup of light or heavy chains. The same framework may be used for several different humanized antibodies (Carter et al., Proc. Natl. Acad. Sci. USA, 89:4285 (1992); Presta et al., J. Immunol. 151:2623 (1993)).

It is further important that antibodies be humanized with retention of high binding affinity for the antigen and other favorable biological properties. To achieve this goal, according to a preferred method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. Inspection of these displays permits analysis of the likely role of the residues in the functioning of the candidate immunoglobulin sequence, i.e., the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Various forms of a humanized anti-GDM antibody antibodies are contemplated. For example, the humanized antibody may be an antibody fragment, such as a Fab, which is optionally conjugated with one or more cytotoxic agent(s) in order to generate an immunoconjugate. Alternatively, the humanized antibody may be an intact antibody, such as an intact IgG1 antibody.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J_{H}) gene in chimeric and germ-line mutant mice results in complete inhibition of endogenous antibody production. Transfer of the human germ-line immunoglobulin gene array into such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggemann et al., Year in Immuno. 7:33 (1993); U.S. Pat. Nos. 5,545,806, 5,569,825, 5,591,669 (all of GenPharm); 5,545,807; and WO 97/17852.

Alternatively, phage display technology (McCafferty et al., Nature 348:552-553 [1990]) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M13 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats, reviewed in, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597 (1991), or Griffith et al., EMBO J. 12:725-734 (1993). See, also, U.S. Pat. Nos. 5,565,332 and 5,573,905.

As discussed above, human antibodies may also be generated by in vitro activated B cells (see U.S. Pat. Nos. 5,567,610 and 5,229,275).

4. Antibody Fragments

In certain circumstances there are advantages of using antibody fragments, rather than whole antibodies. The smaller size of the fragments allows for rapid clearance, while retaining similar antigen binding specificity of the corresponding full length molecule, and may lead to improved access to solid tumors.

Various techniques have been developed for the production of antibody fragments. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. Fab, Fv and scFv antibody fragments can all be expressed in and secreted from E. coli, thus allowing the facile production of large amounts of these fragments. Antibody fragments can be isolated from the antibody phage libraries discussed above. Alternatively, Fab′-SH fragments can be directly recovered from E. coli and chemically coupled to form F(ab′)₂ fragments (Carter et al., Bio/Technology 10:163-167 (1992)). According to another approach, F(ab′)₂ fragments can be isolated directly from recombinant host cell culture. Fab and F(ab′)₂ fragment with increased in vivo half-life comprising a salvage receptor binding epitope residues are described in U.S. Pat. No. 5,869,046. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. Fv and sFv are the only species with intact combining sites that are devoid of constant regions; thus, they are suitable for reduced nonspecific binding during in vivo use. sFv fusion proteins may be constructed to yield fusion of an effector protein at either the amino or the carboxy terminus of an sFv. See Antibody Engineering, ed. Borrebaeck, supra. The antibody fragment may also be a “linear antibody”, e.g., as described in U.S. Pat. No. 5,641,870 for example. Such linear antibody fragments may be monospecific or bispecific.

5. Bispecific Antibodies

Bispecific antibodies are antibodies that have binding specificities for at least two different epitopes. Exemplary bispecific antibodies may bind separate PDM antigens or to two different epitopes of a particular GDM polypeptide described herein. Other such antibodies may combine the above GDM binding site with a binding site for another protein. Alternatively, an anti-GDM arm may be combined with an arm which binds to a triggering molecule on a leukocyte such as a T-cell receptor molecule (e.g. CD3), or Fc receptors for IgG (FcγR), such as FcγRI (CD64), FcγRII (CD32) and FcγRIII (CD16), so as to focus and localize cellular defense mechanisms to the GDM-expressing cell. Bispecific antibodies may also be used to localize cytotoxic agents to cells which express GDM. These antibodies possess a GDM—binding arm and an arm which binds the cytotoxic agent (e.g., saporin, anti-interferon-α, vinca alkaloid, ricin A chain, methotrexate or radioactive isotope hapten). Bispecific antibodies can be prepared as full length antibodies or antibody fragments (e.g., F(ab′)₂ bispecific antibodies).

WO 96/16673 describes a bispecific anti-ErbB2/anti-FcγRIII antibody and U.S. Pat. No. 5,837,234 discloses a bispecific anti-ErbB2/anti-FcγRI antibody. A bispecific anti-ErbB2/Fcα antibody is shown in WO98/02463. U.S. Pat. No. 5,821,337 teaches a bispecific anti-ErbB2/anti-CD3 antibody.

Methods for making bispecific antibodies are known in the art. Traditional production of full length bispecific antibodies is based on the co-expression of two immunoglobulin heavy chain-light chain pairs, where the two chains have different specificities (Millstein et al., Nature 305:537-539 (1983)). Because of the random assortment of immunoglobulin heavy and light chains, these hybridomas (quadromas) produce a potential mixture of 10 different antibody molecules, of which only one has the correct bispecific structure. Purification of the correct molecule, which is usually done by affinity chromatography steps, is rather cumbersome, and the product yields are low. Similar procedures are disclosed in WO 93/08829, and in Traunecker et al., EMBO J. 10:3655-3659 (1991).

According to a different approach, antibody variable domains with the desired binding specificities (antibody-antigen combining sites) are fused to immunoglobulin constant domain sequences. Preferably, the fusion is with an Ig heavy chain constant domain, comprising at least part of the hinge, C_{H}2, and C_{H}3 regions. It is preferred to have the first heavy-chain constant region (C_{H}1) containing the site necessary for light chain bonding, present in at least one of the fusions. DNAs encoding the immunoglobulin heavy chain fusions and, if desired, the immunoglobulin light chain, are inserted into separate expression vectors, and are co-transfected into a suitable host cell. This provides for greater flexibility in adjusting the mutual proportions of the three polypeptide fragments in embodiments when unequal ratios of the three polypeptide chains used in the construction provide the optimum yield of the desired bispecific antibody. It is, however, possible to insert the coding sequences for two or all three polypeptide chains into a single expression vector when the expression of at least two polypeptide chains in equal ratios results in high yields or when the ratios have no significant affect on the yield of the desired chain combination.

In a preferred embodiment of this approach, the bispecific antibodies are composed of a hybrid immunoglobulin heavy chain with a first binding specificity in one arm, and a hybrid immunoglobulin heavy chain-light chain pair (providing a second binding specificity) in the other arm. It was found that this asymmetric structure facilitates the separation of the desired bispecific compound from unwanted immunoglobulin chain combinations, as the presence of an immunoglobulin light chain in only one half of the bispecific molecule provides for a facile way of separation. This approach is disclosed in WO 94/04690. For further details of generating bispecific antibodies see, for example, Suresh et al., Methods in Enzymology 121:210 (1986).

According to another approach described in U.S. Pat. No. 5,731,168, the interface between a pair of antibody molecules can be engineered to maximize the percentage of heterodimers which are recovered from recombinant cell culture. The preferred interface comprises at least a part of the C_{H}3 domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers.

Bispecific antibodies include cross-linked or “heteroconjugate” antibodies. For example, one of the antibodies in the heteroconjugate can be coupled to avidin, the other to biotin. Such antibodies have, for example, been proposed to target immune system cells to unwanted cells (U.S. Pat. No. 4,676,980), and for treatment of HIV infection (WO 91/00360, WO 92/200373, and EP 03089). Heteroconjugate antibodies may be made using any convenient cross-linking methods. Suitable cross-linking agents are well known in the art, and are disclosed in U.S. Pat. No. 4,676,980, along with a number of cross-linking techniques.

Techniques for generating bispecific antibodies from antibody fragments have also been described in the literature. For example, bispecific antibodies can be prepared using chemical linkage. Brennan et al., Science 229:81 (1985) describe a procedure wherein intact antibodies are proteolytically cleaved to generate F(ab′)₂ fragments. These fragments are reduced in the presence of the dithiol complexing agent, sodium arsenite, to stabilize vicinal dithiols and prevent intermolecular disulfide formation. The Fab′ fragments generated are then converted to thionitrobenzoate (TNB) derivatives. One of the Fab′-TNB derivatives is then reconverted to the Fab′-thiol by reduction with mercaptoethylamine and is mixed with an equimolar amount of the other Fab′-TNB derivative to form the bispecific antibody. The bispecific antibodies produced can be used as agents for the selective immobilization of enzymes.

Recent progress has facilitated the direct recovery of Fab′-SH fragments from E. coli, which can be chemically coupled to form bispecific antibodies. Shalaby et al., J. Exp. Med. 175: 217-225 (1992) describe the production of a fully humanized bispecific antibody F(ab′)₂ molecule. Each Fab′ fragment was separately secreted from E. coli and subjected to directed chemical coupling in vitro to form the bispecific antibody. The bispecific antibody thus formed was able to bind to cells overexpressing the ErbB2 receptor and normal human T cells, as well as trigger the lytic activity of human cytotoxic lymphocytes against human breast tumor targets.

Various techniques for making and isolating bispecific antibody fragments directly from recombinant cell culture have also been described. For example, bispecific antibodies have been produced using leucine zippers. Kostelny et al., J. Immunol. 148(5):1547-1553 (1992). The leucine zipper peptides from the Fos and Jun proteins were linked to the Fab′ portions of two different antibodies by gene fusion. The antibody homodimers were reduced at the hinge region to form monomers and then re-oxidized to form the antibody heterodimers. This method can also be utilized for the production of antibody homodimers. The “diabody” technology described by Hollinger et al., Proc. Natl. Acad. Sci. USA 90:6444-6448 (1993) has provided an alternative mechanism for making bispecific antibody fragments. The fragments comprise a V_{H} connected to a V_{L} by a linker which is too short to allow pairing between the two domains on the same chain. Accordingly, the V_{H} and V_{L} domains of one fragment are forced to pair with the complementary V_{L} and V_{H} domains of another fragment, thereby forming two antigen-binding sites. Another strategy for making bispecific antibody fragments by the use of single-chain Fv (sFv) dimers has also been reported. See Gruber et al., J. Immunol., 152:5368 (1994).

Antibodies with more than two valencies are contemplated. For example, trispecific antibodies can be prepared. Tutt et al., J. Immunol. 147:60 (1991).

6. Heteroconijugate Antibodies

Heteroconjugate antibodies are also within the scope of the present invention. Heteroconjugate antibodies are composed of two covalently joined antibodies. Such antibodies have, for example, been proposed to target immune system cells to unwanted cells [U.S. Pat. No. 4,676,980], and for treatment of HIV infection [WO 91/00360; WO 92/200373; EP 03089]. It is contemplated that the antibodies may be prepared in vitro using known methods in synthetic protein chemistry, including those involving crosslinking agents. For example, immunotoxins may be constructed using a disulfide exchange reaction or by forming a thioether bond. Examples of suitable reagents for this purpose include iminothiolate and methyl-4-mercaptobutyrimidate and those disclosed, for example, in U.S. Pat. No. 4,676,980.

7. Multivalent Antibodies

A multivalent antibody may be internalized (and/or catabolized) faster than a bivalent antibody by a cell expressing an antigen to which the antibodies bind. The antibodies of the present invention can be multivalent antibodies (which are other than of the IgM class) with three or more antigen binding sites (e.g. tetravalent antibodies), which can be readily produced by recombinant expression of nucleic acid encoding the polypeptide chains of the antibody. The multivalent antibody can comprise a dimerization domain and three or more antigen binding sites. The preferred dimerization domain comprises (or consists of) an Fc region or a hinge region. In this scenario, the antibody will comprise an Fc region and three or more antigen binding sites amino-terminal to the Fc region. The preferred multivalent antibody herein comprises (or consists of) three to about eight, but preferably four, antigen binding sites. The multivalent antibody comprises at least one polypeptide chain (and preferably two polypeptide chains), wherein the polypeptide chain(s) comprise two or more variable domains. For instance, the polypeptide chain(s) may comprise VD1-(X1)ₙ-VD2-(X2)ₙ-Fc, wherein VD1 is a first variable domain, VD2 is a second variable domain, Fc is one polypeptide chain of an Fc region, X1 and X2 represent an amino acid or polypeptide, and n is 0 or 1. For instance, the polypeptide chain(s) may comprise: VH-CH1-flexible linker-VH-CH1-Fc region chain; or VH-CH1-VH-CH1-Fc region chain. The multivalent antibody herein preferably further comprises at least two (and preferably four) light chain variable domain polypeptides. The multivalent antibody herein may, for instance, comprise from about two to about eight light chain variable domain polypeptides. The light chain variable domain polypeptides contemplated here comprise a light chain variable domain and, optionally, further comprise a CL domain.

8. Effector Function Engineering

It may be desirable to modify the antibody of the invention with respect to effector function, e.g., so as to enhance antigen-dependent cell-mediated cyotoxicity (ADCC) and/or complement dependent cytotoxicity (CDC) of the antibody. This may be achieved by introducing one or more amino acid substitutions in an Fc region of the antibody. Alternatively or additionally, cysteine residue(s) may be introduced in the Fc region, thereby allowing interchain disulfide bond formation in this region. The homodimeric antibody thus generated may have improved internalization capability and/or increased complement-mediated cell killing and antibody-dependent cellular cytotoxicity (ADCC). See Caron et al., J. Exp Med. 176:1191-1195 (1992) and Shopes, B. J. Immunol. 148:2918-2922 (1992). Homodimeric antibodies with enhanced anti-tumor activity may also be prepared using heterobifunctional cross-linkers as described in Wolff et al., Cancer Research 53:2560-2565 (1993). Alternatively, an antibody can be engineered which has dual Fc regions and may thereby have enhanced complement lysis and ADCC capabilities. See Stevenson et al., Anti-Cancer Drug Design 3:219-230 (1989). To increase the serum half life of the antibody, one may incorporate a salvage receptor binding epitope into the antibody (especially an antibody fragment) as described in U.S. Pat. No. 5,739,277, for example. As used herein, the term “salvage receptor binding epitope” refers to an epitope of the Fc region of an IgG molecule (e.g., IgG₁, IgG₂, IgG₃, or IgG₄) that is responsible for increasing the in vivo serum half-life of the IgG molecule.

9. Immunoconjugates

The invention also pertains to immunoconjugates comprising an antibody conjugated to a cytotoxic agent such as a chemotherapeutic agent, a growth inhibitory agent, a toxin (e.g., an enzymatically active toxin of bacterial., fungal., plant, or animal origin, or fragments thereof), or a radioactive isotope (i.e., a radioconjugate).

a. Chemotherapeutic Agents

Chemotherapeutic agents useful in the generation of such immunoconjugates have been described above. Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes. A variety of radionuclides are available for the production of radioconjugated antibodies. Examples include ²¹²Bi, ¹³¹I, ¹³¹In, ⁹⁰Y, and 186Re. Conjugates of the antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science, 238: 1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026.

Conjugates of an antibody and one or more small molecule toxins, such as a calicheamicin, maytansinoids, a trichothene, and CC1065, and the derivatives of these toxins that have toxin activity, are also contemplated herein.

b. Mavtansine and Mavtansinoids

In one preferred embodiment, an anti-GDM antibody (full length or fragments) of the invention is conjugated to one or more maytansinoid molecules.

Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, the disclosures of which are hereby expressly incorporated by reference.

In an attempt to improve their therapeutic index, maytansine and maytansinoids have been conjugated to antibodies specifically binding to tumor cell antigens. Immunoconjugates containing maytansinoids and their therapeutic use are disclosed, for example, in U.S. Pat. Nos. 5,208,020, 5,416,064 and European Patent EP 0 425 235 B1, the disclosures of which are hereby expressly incorporated by reference. Liu et al., Proc. Natl. Acad. Sci. USA 93:8618-8623 (1996) described immunoconjugates comprising a maytansinoid designated DM1 linked to the monoclonal antibody C242 directed against human colorectal cancer. The conjugate was found to be highly cytotoxic towards cultured colon cancer cells, and showed antitumor activity in an in vivo tumor growth assay. Chari et al., Cancer Research 52:127-131 (1992) describe immunoconjugates in which a maytansinoid was conjugated via a disulfide linker to the murine antibody A7 binding to an antigen on human colon cancer cell lines, or to another murine monoclonal antibody TA.1 that binds the HER-2/neu oncogene. The cytotoxicity of the TA.1-maytansonoid conjugate was tested in vitro on the human breast cancer cell line SK-BR-3, which expresses 3×10⁵ HER-2 surface antigens per cell. The drug conjugate achieved a degree of cytotoxicity similar to the free maytansonid drug, which could be increased by increasing the number of maytansinoid molecules per antibody molecule. The A7-maytansinoid conjugate showed low systemic cytotoxicity in mice.

Anti-GDM antibody-maytansinoid or GDM-binding antibody fragment-maytansinoid conjugates may be prepared by chemically linking an anti-GDM antibody or GDM-binding antibody fragment to a maytansinoid molecule without significantly diminishing the biological activity of either the antibody or the maytansinoid molecule. An average of 3-4 maytansinoid molecules conjugated per antibody molecule has shown efficacy in enhancing cytotoxicity of target cells without negatively affecting the function or solubility of the antibody, although even one molecule of toxin/antibody would be expected to enhance cytotoxicity over the use of naked antibody. Maytansinoids are well known in the art and can be synthesized by known techniques or isolated from natural sources. Suitable maytansinoids are disclosed, for example, in U.S. Pat. No. 5,208,020 and in the other patents and nonpatent publications referred to hereinabove. Preferred maytansinoids are maytansinol and maytansinol analogues modified in the aromatic ring or at other positions of the maytansinol molecule, such as various maytansinol esters.

There are many linking groups known in the art for making antibody- or antibody fragment-maytansinoid conjugates, including, for example, those disclosed in U.S. Pat. No. 5,208,020 or EP Patent 0 425 235 B1, and Chari et al., Cancer Research 52:127-131 (1992). The linking groups include disufide groups, thioether groups, acid labile groups, photolabile groups, peptidase labile groups, or esterase labile groups, as disclosed in the above-identified patents, disulfide and thioether groups being preferred.

Conjugates of the antibody or antibody fragment and maytansinoid may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Particularly preferred coupling agents include N-succinimidyl-3-(2-pyridyldithio) propionate (SPDP) (Carlsson et al., Biochem. J. 173:723-737 [1978]) and N-succinimidyl-4-(2-pyridylthio)pentanoate (SPP) to provide for a disulfide linkage.

The linker may be attached to the maytansinoid molecule at various positions, depending on the type of the link. For example, an ester linkage may be formed by reaction with a hydroxyl group using conventional coupling techniques. The reaction may occur at the C-3 position having a hydroxyl group, the C-14 position modified with hyrdoxymethyl, the C-15 position modified with a hydroxyl group, and the C-20 position having a hydroxyl group. In a preferred embodiment, the linkage is formed at the C-3 position of maytansinol or a maytansinol analogue.

c. Calicheamicin

Another immunoconjugate of interest comprises an anti-GDM antibody or GDM binding antibody fragment conjugated to one or more calicheamicin molecules. The calicheamicin family of antibiotics are capable of producing double-stranded DNA breaks at sub-picomolar concentrations. For the preparation of conjugates of the calicheamicin family, see U.S. Pat. Nos. 5,712,374, 5,714,586, 5,739,116, 5,767,285, 5,770,701, 5,770,710, 5,773,001, 5,877,296 (all to American Cyanamid Company). Structural analogues of calicheamicin which may be used include, but are not limited to, γhd i^{I}, α₂^{I}, α₃^{I}, N-acetyl-γ₁^{I}, PSAG and θ^{I}₁(Hinman et al., Cancer Research 53:3336-3342 (1993), Lode et al., Cancer Research 58:2925-2928 (1998) and the aforementioned U.S. patents to American Cyanamid). Another anti-tumor drug that the antibody can be conjugated is QFA which is an antifolate. Both calicheamicin and QFA have intracellular sites of action and do not readily cross the plasma membrane. Therefore, cellular uptake of these agents through antibody mediated internalization greatly enhances their cytotoxic effects.

d. Other Cytotoxic Agents

Other antitumor agents that can be conjugated to the anti-GDM antibodies of the invention include BCNU, streptozoicin, vincristine and 5-fluorouracil, the family of agents known collectively LL-E33288 complex described in U.S. Pat. Nos. 5,053,394, 5,770,710, as well as esperamicins (U.S. Pat. No. 5,877,296).

Enzymatically active toxins and fragments thereof which can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin and the tricothecenes. See, for example, WO 93/21232 published Oct. 28, 1993.

The present invention further contemplates an immunoconjugate formed between an antibody and a compound with nucleolytic activity (e.g., a ribonuclease or a DNA endonuclease such as a deoxyribonuclease; DNase).

For selective destruction of the tumor, the antibody may comprise a highly radioactive atom. A variety of radioactive isotopes are available for the production of radioconjugated anti-GDM antibodies. Examples include At²¹¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the conjugate is used for diagnosis, it may comprise a radioactive atom for scintigraphic studies, for example tc^{99m} or I¹²³, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

The radio—or other labels may be incorporated in the conjugate in known ways. For example, the peptide may be biosynthesized or may be synthesized by chemical amino acid synthesis using suitable amino acid precursors involving, for example, fluorine-19 in place of hydrogen. Labels such as tc^{99m} or I¹²³, .Re¹⁸⁶, Re¹⁸⁸ and In¹¹¹ can be attached via a cysteine residue in the peptide. Yttrium-90 can be attached via a lysine residue. The IODOGEN method (Fraker et al (1978) Biochem. Biophys. Res. Commun. 80: 49-57 can be used to incorporate iodine-123. “Monoclonal Antibodies in Immunoscintigraphy” (Chatal., CRC Press 1989) describes other methods in detail.

Conjugates of the antibody and cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate, iminothiolane (IT), bifunctional derivatives of imidoesters (such as dimethyl adipimidate HCL), active esters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of the cytotoxic drug in the cell. For example, an acid-labile linker, peptidase-sensitive linker, photolabile linker, dimethyl linker or disulfide-containing linker (Chari et al., Cancer Research 52:127-131 (1992); U.S. Pat. No. 5,208,020) may be used.

Alternatively, a fusion protein comprising the anti-GDM antibody or GDM binding antibody fragment and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

In yet another embodiment, the antibody may be conjugated to a “receptor” (such streptavidin) for utilization in tumor pre-targeting wherein the antibody-receptor conjugate is administered to the patient, followed by removal of unbound conjugate from the circulation using a clearing agent and then administration of a “ligand” (e.g., avidin) which is conjugated to a cytotoxic agent (e.g., a radionucleotide).

10. Immunoliposomes

The anti-GDM antibodies or GDM binding antibody fragment disclosed herein may also be formulated as immunoliposomes. A “liposome” is a small vesicle composed of various types of lipids, phospholipids and/or surfactant which is useful for delivery of a drug to a mammal. The components of the liposome are commonly arranged in a bilayer formation, similar to the lipid arrangement of biological membranes. Liposomes containing the antibody are prepared by methods known in the art, such as described in Epstein et al., Proc. Natl. Acad. Sci. USA 82:3688 (1985); Hwang et al., Proc. Natl Acad. Sci. USA 77:4030 (1980); U.S. Pat. Nos. 4,485,045 and 4,544,545; and WO97/38731 published Oct. 23, 1997. Liposomes with enhanced circulation time are disclosed in U.S. Pat. No. 5,013,556.

Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter. Fab′ fragments of the antibody of the present invention can be conjugated to the liposomes as described in Martin et al., J. Biol. Chem. 257:286-288 (1982) via a disulfide interchange reaction. A chemotherapeutic agent is optionally contained within the liposome. See Gabizon et al., J. National Cancer Inst. 81(19):1484 (1989).

B. GDM Binding Oligopeptides

GDM binding oligopeptides of the present invention are oligopeptides that bind, preferably specifically, to a GDM polypeptide as described herein. GDM binding oligopeptides may be chemically synthesized using known oligopeptide synthesis methodology or may be prepared and purified using recombinant technology. GDM binding oligopeptides are usually at least about 5 amino acids in length, alternatively at least about 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 amino acids in length or more, wherein such oligopeptides that are capable of binding, preferably specifically, to a GDM polypeptide as described herein. GDM binding oligopeptides may be identified without undue experimentation using well known techniques. In this regard, it is noted that techniques for screening oligopeptide libraries for oligopeptides that are capable of specifically binding to a polypeptide target are well known in the art (see, e.g., U.S. Pat. Nos. 5,556,762, 5,750,373, 4,708,871, 4,833,092, 5,223,409, 5,403,484, 5,571,689, 5,663,143; PCT Publication Nos. WO 84/03506 and WO84/03564; Geysen et al. Proc. Natl. Acad. Sci. U.S.A., 81:3998-4002 (1984); Geysen et al., Proc. Natl. Acad. Sci. U.S.A., 82:178-182 (1985); Geysen et al., in Synthetic Peptides as Antigens, 130-149 (1986); Geysen et al. J. Immunol. Meth., 102:259-274 (1987); Schoofs et al., J. Immunol., 140:611-616 (1988), Cwirla, S. E. et al. (1990) Proc. Natl. Acad. Sci. USA, 87:6378; Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363, and Smith, G. P. (1991) Current Opin. Biotechnol., 2:668).

In this regard, bacteriophage (phage) display is one well known technique which allows one to screen large oligopeptide libraries to identify member(s) of those libraries which are capable of specifically binding to a polypeptide target. Phage display is a technique by which variant polypeptides are displayed as fusion proteins to the coat protein on the surface of bacteriophage particles (Scott, J. K. and Smith, G. P. (1990) Science 249: 386). The utility of phage display lies in the fact that large libraries of selectively randomized protein variants (or randomly cloned cDNAs) can be rapidly and efficiently sorted for those sequences that bind to a target molecule with high affinity. Display of peptide (Cwirla, S. E. et al. (1990) Proc. Natl. Acad. Sci. USA, 87:6378) or protein (Lowman, H. B. et al. (1991) Biochemistry, 30:10832; Clackson, T. et al. (1991) Nature, 352: 624; Marks, J. D. et al. (1991), J. Mol. Biol., 222:581; Kang, A. S. et al. (1991) Proc. Natl. Acad. Sci. USA, 88:8363) libraries on phage have been used for screening millions of polypeptides or oligopeptides for ones with specific binding properties (Smith, G. P. (1991) Current Opin. Biotechnol., 2:668). Sorting phage libraries of random mutants requires a strategy for constructing and propagating a large number of variants, a procedure for affinity purification using the target receptor, and a means of evaluating the results of binding enrichments. U.S. Pat. Nos. 5,223,409, 5,403,484, 5,571,689, and 5,663,143.

Although most phage display methods have used filamentous phage, lambdoid phage display systems (WO 95/34683; U.S. Pat. No. 5,627,024), T4 phage display systems (Ren et al., Gene, 215: 439 (1998); Zhu et al., Cancer Research, 58(15): 3209-3214 (1998); Jiang et al., Infection & Immunity, 65(11): 4770-4777 (1997); Ren et al., Gene, 195(2):303-311 (1997); Ren, Protein Sci., 5: 1833 (1996); Efimov et al., Virus Genes, 10: 173 (1995)) and T7 phage display systems (Smith and Scott, Methods in Enzymology, 217: 228-257 (1993); U.S. Pat. No. 5,766,905) are also known.

Many other improvements and variations of the basic phage display concept have now been developed. These improvements enhance the ability of display systems to screen peptide libraries for binding to selected target molecules and to display functional proteins with the potential of screening these proteins for desired properties. Combinatorial reaction devices for phage display reactions have been developed (WO 98/14277) and phage display libraries have been used to analyze and control bimolecular interactions (WO 98/20169; WO 98/20159) and properties of constrained helical peptides (WO 98/20036). WO 97/35196 describes a method of isolating an affinity ligand in which a phage display library is contacted with one solution in which the ligand will bind to a target molecule and a second solution in which the affinity ligand will not bind to the target molecule, to selectively isolate binding ligands. WO 97/46251 describes a method of biopanning a random phage display library with an affinity purified antibody and then isolating binding phage, followed by a micropanning process using microplate wells to isolate high affinity binding phage. The use of Staphlylococcus aureus protein A as an affinity tag has also been reported (Li et al. (1998) Mol Biotech., 9:187). WO 97/47314 describes the use of substrate subtraction libraries to distinguish enzyme specificities using a combinatorial library which may be a phage display library. A method for selecting enzymes suitable for use in detergents using phage display is described in WO 97/09446. Additional methods of selecting specific binding proteins are described in U.S. Pat. Nos. 5,498,538, 5,432,018, and WO 98/15833.

Methods of generating peptide libraries and screening these libraries are also disclosed in U.S. Pat. Nos. 5,723,286, 5,432,018, 5,580,717, 5,427,908, 5,498,530, 5,770,434, 5,734,018, 5,698,426, 5,763,192, and 5,723,323.

C. GDM Small Molecule Antagonists

GDM small molecule antagonists are small molecules other than oligopeptides or antibodies (or fragments thereof) as defined herein that bind, preferably specifically, to a signaling component (e.g., receptor, ligand, intacellular component, etc.) of a GDM polypeptide as described herein. Such organic molecules may be identified and chemically synthesized using known methodology (see, e.g., PCT Publication Nos. WO00/00823 and WO00/39585). Such organic molecules are usually less than about 2000 daltons in size, alternatively less than about 1500, 750, 500, 250 or 200 daltons in size, wherein such organic molecules that are capable of binding, preferably specifically, to a GDM polypeptide as described herein may be identified without undue experimentation using well known techniques. In this regard, it is noted that techniques for screening organic molecule libraries for molecules that are capable of binding to a polypeptide target are well known in the art (see, e.g., PCT Publication Nos. WO00/00823 and WO00/39585). GDM molecule antagonists may be, for example, aldehydes, ketones, oximes, hydrazones, semicarbazones, carbazides, primary amines, secondary amines, tertiary amines, N-substituted hydrazines, hydrazides, alcohols, ethers, thiols, thioethers, disulfides, carboxylic acids, esters, amides, ureas, carbamates, carbonates, ketals, thioketals, acetals, thioacetals, aryl halides, aryl sulfonates, alkyl halides, alkyl sulfonates, aromatic compounds, heterocyclic compounds, anilines, alkenes, alkynes, diols, amino alcohols, oxazolidines, oxazolines, thiazolidines, thiazolines, enamines, sulfonamides, epoxides, aziridines, isocyanates, sulfonyl chlorides, diazo compounds, acid chlorides, or the like.

D. Screening for GDM Antagonists

Techniques for generating the antibodies, polypeptides, oligopeptides and organic molecules of the invention have been described above. One may further select antibodies (and antigen-binding fragments thereof), oligopeptides or other organic molecules with certain biological characteristics, as desired.

The growth inhibitory effects of the various GDM antagonists useable in the invention may be assessed by methods known in the art, e.g., using glioma cells which express a GDM polypeptide either endogenously or following transfection with the GDM gene. For example, appropriate tumor cell lines and cells transfected with GDM-encoding nucleic may be treated with the GDM antagonists of the invention at various concentrations for a few days (e.g., 2-7) days and stained with crystal violet or MTT or analyzed by some other colorimetric assay. Another method of measuring proliferation would be by comparing ³H-thymidine uptake by the cells treated in the presence or absence of such GDM antagonists. After treatment, the cells are harvested and the amount of radioactivity incorporated into the DNA quantitated in a scintillation counter. Appropriate positive controls include treatment of a selected cell line with a growth inhibitory antibody known to inhibit growth of that cell line. Growth inhibition of tumor cells in vivo can be determined in various ways known in the art. Preferably, the tumor cell is one that overexpresses a GDM polypeptide. Preferably, such GDM antagonists will inhibit cell proliferation of a GDM-expressing tumor cell in vitro or in vivo by about 25-100% compared to the untreated tumor cell, more preferably, by about 30-100%, and even more preferably by about 50-100% or 70-100%, in one embodiment, at an antibody concentration of about 0.5 to 30 μg/ml. Growth inhibition can be measured at a GDM antagonist concentration of about 0.5 to 30 μg/ml or about 0.5 nM to 200 nM in cell culture, where the growth inhibition is determined 1-10 days after exposure of the tumor cells to the antibody. The antibody is growth inhibitory in vivo if administration of antagonist and/or agonist at about 1 μg/kg to about 100 mg/kg body weight results in reduction in tumor size or reduction of tumor cell proliferation within about 5 days to 3 months from the first administration of the antibody, preferably within about 5 to 30 days.

To select for GDM antagonists which induces cell death, loss of membrane integrity as indicated by, e.g., propidium iodide (PI), trypan blue or 7AAD uptake may be assessed relative to control. A PI uptake assay can be performed in the absence of complement and immune effector cells. GDM polypeptide-expressing tumor cells are incubated with medium alone or medium containing the appropriate GDM antagonist. The cells are incubated for a 3 day time period. Following each treatment, cells are washed and aliquoted a into 35 mm strainer-capped 12×75 tubes (1 ml per tube, 3 tubes per treatment group) for removal of cell clumps. Tubes then receive PI (10 μg/ml). Samples may be analyzed using a FACSCAN® flow cytometer and FACSCONVERT® CellQuest software (Becton Dickinson). Those GDM antagonist that induce statistically significant levels of cell death as determined by PI uptake may then be selected.

To screen for GDM antagonists which bind to an epitope on a GDM polypeptide bound by an antibody of interest, a routine cross-blocking assay such as that described in Antibodies: A Laboratory Manual, Cold Spring Harbor Laboratory, Ed Harlow and David Lane (1988), can be performed. This assay can be used to determine if a test antibody, oligopeptide or other organic molecule binds the same site or epitope as a known anti-GDM antibody. Alternatively, or additionally, epitope mapping can be performed by methods known in the art. For example, the antibody sequence can be mutagenized such as by alanine scanning, to identify contact residues. The mutant antibody is initially tested for binding with polyclonal antibody to ensure proper folding. In a different method, peptides corresponding to different regions of a GDM polypeptide can be used in competition assays with the test antibodies or with a test antibody and an antibody with a characterized or known epitope.

E. Antibody Dependent Enzyme Mediated Prodrug Therapy (ADEPT)

The GDM antagonist antibodies of the present invention may also be used in ADEPT by conjugating the antibody to a prodrug-activating enzyme which converts a prodrug (e.g., a peptidyl chemotherapeutic agent, see WO81/01145) to an active anti-cancer drug. See, for example, WO 88/07378 and U.S. Pat. No. 4,975,278.

The enzyme component of the immunoconjugate useful for ADEPT includes any enzyme capable of acting on a prodrug in such a way so as to covert it into its more active, cytotoxic form.

Enzymes that are useful in the method of this invention include, but are not limited to, alkaline phosphatase useful for converting phosphate-containing prodrugs into free drugs; arylsulfatase useful for converting sulfate-containing prodrugs into free drugs; cytosine deaminase useful for converting non-toxic 5-fluorocytosine into the anti-cancer drug, 5-fluorouracil; proteases, such as serratia protease, thermolysin, subtilisin, carboxypeptidases and cathepsins (such as cathepsins B and L), that are useful for converting peptide-containing prodrugs into free drugs; D-alanylcarboxypeptidases, useful for converting prodrugs that contain D-amino acid substituents; carbohydrate-cleaving enzymes such as β-galactosidase and neuraminidase useful for converting glycosylated prodrugs into free drugs; β-lactamase useful for converting drugs derivatized with β-lactams into free drugs; and penicillin amidases, such as penicillin V amidase or penicillin G amidase, useful for converting drugs derivatized at their amine nitrogens with phenoxyacetyl or phenylacetyl groups, respectively, into free drugs. Alternatively, antibodies with enzymatic activity, also known in the art as “abzymes”, can be used to convert the prodrugs of the invention into free active drugs (see, e.g., Massey, Nature 328:457-458 (1987)). Antibody-abzyme conjugates can be prepared as described herein for delivery of the abzyme to a tumor cell population.

The enzymes of this invention can be covalently bound to the anti-GDM antibodies by techniques well known in the art such as the use of the heterobifunctional crosslinking reagents discussed above. Alternatively, fusion proteins comprising at least the antigen binding region of an antibody of the invention linked to at least a functionally active portion of an enzyme of the invention can be constructed using recombinant DNA techniques well known in the art (see, e.g., Neuberger et al., Nature 312:604-608 (1984).

F. GDM Polypeptides Variants

In addition to the GDM polypeptides described herein, it is contemplated that variants of such molecules can be prepared for use with the invention herein. Such variants can be prepared by introducing appropriate nucleotide changes into the encoding DNA, and/or by synthesis of the desired antibody or polypeptide. Those skilled in the art will appreciate that amino acid changes may alter post-translational processes of these molecules, such as changing the number or position of glycosylation sites or altering the membrane anchoring characteristics.

Variations in amino acid sequence can be made, for example, using any of the techniques and guidelines for conservative and non-conservative mutations set forth, for instance, in U.S. Pat. No. 5,364,934. Variations may be a substitution, deletion or insertion of one or more codons encoding the amino acid sequence that results in a change in the amino acid sequence as compared with the native sequence. Optionally the variation is by substitution of at least one amino acid with any other amino acid in one or more of the domains of the amino acid sequence of interest. Guidance in determining which amino acid residue may be inserted, substituted or deleted without adversely affecting the desired activity may be found by comparing the sequence of the amino acid sequence of interest with homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence.

Fragments of the various PDM polypeptides are provided herein. Such fragments may be truncated at the N-terminus or C-terminus, or may lack internal residues, for example, when compared with a full length native antibody or protein. Such fragments which lack amino acid residues that are not essential for a desired biological activity are also useful with the disclosed methods.

The above polypeptide fragments may be prepared by any of a number of conventional techniques. Desired peptide fragments may be chemically synthesized. An alternative approach involves generating such fragments by enzymatic digestion, e.g., by treating the protein with an enzyme known to cleave proteins at sites defined by particular amino acid residues, or by digesting the DNA with suitable restriction enzymes and isolating the desired fragment. Yet another suitable technique involves isolating and amplifying a DNA fragment encoding the desired fragment fragment by polymerase chain reaction (PCR). Oligonucleotides that define the desired termini of the DNA fragment are employed at the 5′ and 3′ primers in the PCR. Preferably, such fragments share at least one biological and/or immunological activity with the corresponding full length molecule.

In particular embodiments, conservative substitutions of interest are shown in Table 6 under the heading of preferred substitutions. If such substitutions result in a change in biological activity, then more substantial changes, denominated exemplary substitutions in Table 6, or as further described below in reference to amino acid classes, are introduced and the products screened in order to identify the desired variant.

TABLE 6

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser, Ala | Ser |
| Gln (Q) | Asn; Glu | Asn |
| Glu (E) | Asp, Gln | Asp |
| Gly (G) | Pro; Ala | Ala |
| His (H) | Asn; Gln; Lys; Arg | Arg |
| Ile (I) | Leu; Val; Met; Ala; Phe; | Leu |
| | Norleucine | |
| Leu (L) | Norleucine; Ile; Val; | Ile |
| | Met; Ala; Phe | |
| Lys (K) | Arg; Gln; Asn | Arg |
| Met (M) | Leu; Phe; Ile | Leu |
| Phe (F) | Trp; Leu; Val; Ile; Ala; Tyr | Leu |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; | Leu |
| | Ala; Norleucine | |

Substantial modifications in function or immunological identity of the GDM polypeptides are accomplished by selecting substitutions that differ significantly in their effect on maintaining (a) the structure of the polypeptide backbone in the area of the substitution, for example, as a sheet or helical conformation, (b) the charge or hydrophobicity of the molecule at the target site, or (c) the bulk of the side chain. Naturally occurring residues are divided into groups based on common side-chain properties:
(1) hydrophobic: Norleucine, Met, Ala, Val., Leu, Ile;
(2) neutral hydrophilic: Cys, Ser, Thr; Asn; Gln
(3) acidic: Asp, Glu;
(4) basic: His, Lys, Arg;
(5) residues that influence chain orientation: Gly, Pro; and
(6) aromatic: Trp, Tyr, Phe.

Non-conservative substitutions will entail exchanging a member of one of these classes for another class. Such substituted residues also may be introduced into the conservative substitution sites or, more preferably, into the remaining (non-conserved) sites.

The variations can be made using methods known in the art such as oligonucleotide-mediated (site-directed) mutagenesis, alanine scanning, and PCR mutagenesis. Site-directed mutagenesis [Carter et al., Nucl. Acids Res., 13:4331 (1986); Zoller et al., Nucl. Acids Res., 10:6487 (1987)], cassette mutagenesis [Wells et al., Gene, 34:315 (1985)], restriction selection mutagenesis [Wells et al., Philos. Trans. R. Soc. London SerA, 317:415 (1986)] or other known techniques can be performed on the cloned DNA to produce the anti-GDM molecule.

Scanning amino acid analysis can also be employed to identify one or more amino acids along a contiguous sequence. Among the preferred scanning amino acids are relatively small, neutral amino acids. Such amino acids include alanine, glycine, serine, and cysteine. Alanine is typically a preferred scanning amino acid among this group because it eliminates the side-chain beyond the beta-carbon and is less likely to alter the main-chain conformation of the variant [Cunningham and Wells, Science, 244:1081-1085 (1989)]. Alanine is also typically preferred because it is the most common amino acid. Further, it is frequently found in both buried and exposed positions [Creighton, The Proteins, (W. H. Freeman & Co., N.Y.); Chothia, J. Mol. Biol., 150:1 (1976)]. If alanine substitution does not yield adequate amounts of variant, an isoteric amino acid can be used.

Any cysteine residue not involved in maintaining the proper conformation of the GDM polypeptides also may be substituted, generally with serine, to improve the oxidative stability of the molecule and prevent aberrant crosslinking. Conversely, cysteine bond(s) may be added to such a molecule to improve its stability (particularly where the antibody is an antibody fragment such as an Fv fragment).

A particularly preferred type of substitutional variant involves substituting one or more hypervariable region residues of a parent antibody (e.g., a humanized or human antibody). Generally, the resulting variant(s) selected for further development will have improved biological properties relative to the parent antibody from which they are generated. A convenient way for generating such substitutional variants involves affinity maturation using phage display. Briefly, several hypervariable region sites (e.g., 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g., binding affinity) as herein disclosed. In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and target polypeptide. Such contact residues and neighboring residues are candidates for substitution according to the techniques elaborated herein. Once such variants are generated, the panel of variants is subjected to screening as described herein and antibodies with superior properties in one or more relevant assays may be selected for further development.

Nucleic acid molecules encoding amino acid sequence variants of GDM polypeptides are prepared by a variety of methods known in the art. These methods include, but are not limited to, isolation from a natural source (in the case of naturally occurring amino acid sequence variants) or preparation by oligonucleotide-mediated (or site-directed) mutagenesis, PCR mutagenesis, and cassette mutagenesis of a native sequence or an earlier prepared variant.

G. Modifications of GDM Polypeptides

The GDM that have been covalently modified may also be suitable for use within the scope of this invention. One type of covalent modification includes reacting targeted amino acid residues of such antibodies and polypeptides with an organic derivatizing agent that is capable of reacting with selected side chains or the N- or C- terminal residues of such antibodies and polypeptides. Derivatization with bifunctional agents is useful, for instance, for crosslinking the preceding molecules to a water-insoluble support matrix or surface for use in purification. Commonly used crosslinking agents include, e.g., 1,1-bis(diazoacetyl)-2-phenylethane, glutaraldehyde, N-hydroxysuccinimide esters, for example, esters with 4-azidosalicylic acid, homobifunctional imidoesters, including disuccinimidyl esters such as 3,3′-dithiobis(succinimidylpropionate), bifunctional maleimides such as bis-N-maleimido-1,8-octane and agents such as methyl-3-[(p-azidophenyl) dithio]propioimidate.

Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the a-amino groups of lysine, arginine, and histidine side chains [T. E. Creighton, Proteins: Structure and Molecular Properties, W. H. Freeman & Co., San Francisco, pp. 79-86 (1983)], acetylation of the N-terminal amine, and amidation of any C-terminal carboxyl group.

Another type of covalent modification of the GDM polypeptides comprises altering the native glycosylation pattern of the antibody or polypeptide. “Altering the native glycosylation pattern” is intended for purposes herein to mean deleting one or more carbohydrate moieties found in native sequence (either by removing the underlying glycosylation site or by deleting the glycosylation by chemical and/or enzymatic means), and/or adding one or more glycosylation sites that are not present in the respective native sequence. In addition, the phrase includes qualitative changes in the glycosylation of the native proteins, involving a change in the nature and proportions of the various carbohydrate moieties present.

Glycosylation of antibodies and other polypeptides is typically either N-linked or O-linked. N-linked refers to the attachment of the carbohydrate moiety to the side chain of an asparagine residue. The tripeptide sequences asparagine-X-serine and asparagine-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. O-linked glycosylation refers to the attachment of one of the sugars N-aceylgalactosamine, galactose, or xylose to a hydroxyamino acid, most commonly serine or threonine, although 5-hydroxyproline or 5-hydroxylysine may also be used.

Addition of glycosylation sites may be accomplished by altering the amino acid sequence such that it contains one or more of the above-described tripeptide sequences (for N-linked glycosylation sites). The alteration may also be made by the addition of, or substitution by, one or more serine or threonine residues to the sequence of the original such antibody or polypeptide (for O-linked glycosylation sites). Such antibody or polypeptide sequence may optionally be altered through changes at the DNA level, particularly by mutating the DNA encoding the preceding amino acid sequences at preselected bases such that codons are generated that will translate into the desired amino acids.

Another means of increasing the number of carbohydrate moieties is by chemical or enzymatic coupling of glycosides to the polypeptide. Such methods are described in the art, e.g., in WO 87/05330 published 11 Sep. 1987, and in Aplin and Wriston, CRC Crit. Rev. Biochem., pp. 259-306 (1981).

Removal of carbohydrate moieties may be accomplished chemically or enzymatically or by mutational substitution of codons encoding for amino acid residues that serve as targets for glycosylation. Chemical deglycosylation techniques are known in the art and described, for instance, by Hakimuddin, et al., Arch. Biochem. Biophys., 259:52 (1987) and by Edge et al., Anal. Biochem., 118:131 (1981). Enzymatic cleavage of carbohydrate moieties on polypeptides can be achieved by the use of a variety of endo- and exo-glycosidases as described by Thotakura et al., Meth. Enzymol., 138:350 (1987).

Another type of covalent modification comprises linking to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337. The GDM polypeptides also may be entrapped in microcapsules prepared, for example, by coacervation techniques or by interfacial polymerization (for example, hydroxymethylcellulose or gelatin-microcapsules and poly-(methylmethacylate) microcapsules, respectively), in colloidal drug delivery systems (for example, liposomes, albumin microspheres, microemulsions, nano-particles and nanocapsules), or in macroemulsions. Such techniques are disclosed in Remington's Pharmaceutical Sciences, 16th edition, Oslo, A., Ed., (1980).

Modifications forming chimeric molecules results from fusions of GDM polypeptides to another, heterologous polypeptide or amino acid sequence are contemplated for use with the present methods.

In one embodiment, such a chimeric molecule comprises a fusion of the GDM polypeptides with a tag polypeptide which provides an epitope to which an anti-tag antibody can selectively bind. The epitope tag is generally placed at the amino- or carboxyl-terminus of such antibody or polypeptide. The presence of such epitope-tagged forms of such antibodies or polypeptides can be detected using an antibody against the tag polypeptide. Also, provision of the epitope tag enables such antibodies or polypeptide to be readily purified by affinity purification using an anti-tag antibody or another type of affinity matrix that binds to the epitope tag. Various tag polypeptides and their respective antibodies are well known in the art. Examples include poly-histidine (poly-his) or poly-histidine-glycine (poly-his-gly) tags; the flu HA tag polypeptide and its antibody 12CA5 [Field et al., Mol. Cell. Biol., 8:2159-2165 (1988)]; the c-myc tag and the 8F9, 3C7, 6E10, G4, B7 and 9E10 antibodies thereto [Evan et al., Molecular and Cellular Biology, 5:3610-3616 (1985)]; and the Herpes Simplex virus glycoprotein D (gD) tag and its antibody [Paborsky et al., Protein Engineering, 3(6):547-553 (1990)]. Other tag polypeptides include the Flag-peptide [Hopp et al., BioTechnology, 6:1204-1210 (1988)]; the KT3 epitope peptide [Martin et al., Science, 255:192-194 (1992)]; an α-tubulin epitope peptide [Skinner et al., J. Biol. Chem., 266:15163-15166 (1991)]; and the T7 gene 10 protein peptide tag [Lutz-Freyermuth et al., Proc. Natl. Acad. Sci. USA, 87:6393-6397 (1990)].

In an alternative embodiment, the chimeric molecule may comprise a fusion of the GDM polypeptides with an immunoglobulin or a particular region of an immunoglobulin. For a bivalent form of the chimeric molecule (also referred to as an “immunoadhesin”), such a fusion could be to the Fc region of an IgG molecule. The Ig fusions preferably include the substitution of a soluble (transmembrane domain deleted or inactivated) form of a preceding antibody or polypeptide in the place of at least one variable region within an Ig molecule. In a particularly preferred embodiment, the immunoglobulin fusion includes the hinge, CH₂ and CH₃, or the hinge, CH₁, CH₂ and CH₃ regions of an IgG1 molecule. For the production of immunoglobulin fusions see also US Pat. No. 5,428,130 issued Jun. 27, 1995.

H. Preparation of GDM Polypeptides

The description below relates primarily to production of GDM polypeptides by culturing cells transformed or transfected with a vector containing nucleic acid such antibodies, polypeptides and oligopeptides. It is, of course, contemplated that alternative methods, which are well known in the art, may be employed to prepare such antibodies, polypeptides and oligopeptides. For instance, the appropriate amino acid sequence, or portions thereof, may be produced by direct peptide synthesis using solid-phase techniques [see, e.g., Stewart et al., Solid-Phase Peptide Synthesis, W. H. Freeman Co., San Francisco, Calif. (1969); Merrifield, J. Am. Chem. Soc., 85:2149-2154 (1963)]. In vitro protein synthesis may be performed using manual techniques or by automation. Automated synthesis may be accomplished, for instance, using an Applied Biosystems Peptide Synthesizer (Foster City, Calif.) using manufacturer's instructions. Various portions of such antibodies, polypeptides or oligopeptides may be chemically synthesized separately and combined using chemical or enzymatic methods to produce the desired product.

1. Isolation of DNA Encoding GDM Polypeptides

DNA encoding a GDM polypeptide may be obtained from a cDNA library prepared from tissue believed to possess such antibody, polypeptide or oligopeptide mRNA and to express it at a detectable level. Accordingly, DNA encoding such polypeptides can be conveniently obtained from a cDNA library prepared from human tissue, a genomic library or by known synthetic procedures (e.g., automated nucleic acid synthesis).

Libraries can be screened with probes (such as oligonucleotides of at least about 20-80 bases) designed to identify the gene of interest or the protein encoded by it. Screening the cDNA or genomic library with the selected probe may be conducted using standard procedures, such as described in Sambrook et al., Molecular Cloning: A Laboratory Manual (New York: Cold Spring Harbor Laboratory Press, 1989). Alternatively, PCR methodology may be used. [Sambrook et al., supra; Dieffenbach et al., PCR Primer: A Laboratory Manual (Cold Spring Harbor Laboratory Press, 1995)].

Techniques for screening a cDNA library are well known in the art. The oligonucleotide sequences selected as probes should be of sufficient length and sufficiently unambiguous that false positives are minimized. The oligonucleotide is preferably labeled such that it can be detected upon hybridization to DNA in the library being screened. Methods of labeling are well known in the art, and include the use of radiolabels like ³²P-labeled ATP, biotinylation or enzyme labeling. Hybridization conditions, including moderate stringency and high stringency, are provided in Sambrook et al., supra.

Sequences identified in such library screening methods can be compared and aligned to other known sequences deposited and available in public databases such as GenBank or other private sequence databases. Sequence identity (at either the amino acid or nucleotide level) within defined regions of the molecule or across the full-length sequence can be determined using methods known in the art and as described herein.

Nucleic acid having protein coding sequence may be obtained by screening selected cDNA or genomic libraries using the deduced amino acid sequence disclosed herein for the first time, and, if necessary, using conventional primer extension procedures as described in Sambrook et al., supra, to detect precursors and processing intermediates of mRNA that may not have been reverse-transcribed into cDNA.

2. Selection and Transformation of Host Cells

Host cells are transfected or transformed with expression or cloning vectors described herein for GDM polypeptide production and cultured in conventional nutrient media modified as appropriate for inducing promoters, selecting transformants, or amplifying the genes encoding the desired sequences. The culture conditions, such as media, temperature, pH and the like, can be selected by the skilled artisan without undue experimentation. In general, principles, protocols, and practical techniques for maximizing the productivity of cell cultures can be found in Mammalian Cell Biotechnology: A Practical Approach, M. Butler, ed. (IRL Press, 1991) and Sambrook et al., supra.

Methods of eukaryotic cell transfection and prokaryotic cell transformation are known to the ordinarily skilled artisan, for example, CaCl₂, CaPO₄, liposome-mediated and electroporation. Depending on the host cell used, transformation is performed using standard techniques appropriate to such cells. The calcium treatment employing calcium chloride, as described in Sambrook et al., supra, or electroporation is generally used for prokaryotes. Infection with Agrobacterium tumefaciens is used for transformation of certain plant cells, as described by Shaw et al., Gene, 23:315 (1983) and WO 89/05859 published 29 Jun. 1989. For mammalian cells without such cell walls, the calcium phosphate precipitation method of Graham and van der Eb, Virology, 52:456-457 (1978) can be employed. General aspects of mammalian cell host system transfections have been described in U.S. Pat. No. 4,399,216. Transformations into yeast are typically carried out according to the method of Van Solingen et al., J. Bact., 130:946 (1977) and Hsiao et al., Proc. Natl. Acad. Sci. (USA), 76:3829 (1979). However, other methods for introducing DNA into cells, such as by nuclear microinjection, electroporation, bacterial protoplast fusion with intact cells, or polycations, e.g., polybrene, polyomithine, may also be used. For various techniques for transforming mammalian cells, see Keown et al., Methods in Enzymology, 185:527-537 (1990) and Mansour et al., Nature, 336:348-352 (1988).

Suitable host cells for cloning or expressing the DNA in the vectors herein include prokaryote, yeast, or higher eukaryote cells. Suitable prokaryotes include but are not limited to eubacteria, such as Gram-negative or Gram-positive organisms, for example, Enterobacteriaceae such as E. coli. Various E. coli strains are publicly available, such as E. coli K12 strain MM294 (ATCC 31,446); E. coli X1776 (ATCC 31,537); E. coli strain W3110 (ATCC 27,325) and K5 772 (ATCC 53,635). Other suitable prokaryotic host cells include Enterobacteriaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. Iicheniformis (e.g., B. Iicheniformis 41P disclosed in DD 266,710 published 12 Apr. 1989), Pseudomonas such as P. aeruginosa, and Streptomyces. These examples are illustrative rather than limiting. Strain W3 110 is one particularly preferred host or parent host because it is a common host strain for recombinant DNA product fermentations. Preferably, the host cell secretes minimal amounts of proteolytic enzymes. For example, strain W3110 may be modified to effect a genetic mutation in the genes encoding proteins endogenous to the host, with examples of such hosts including E. coli W3110 strain 1A2, which has the complete genotype tonA ; E. coli W3110 strain 9E4, which has the complete genotype tonA ptr3; E. coli W3110 strain 27C7 (ATCC 55,244), which has the complete genotype tonA ptr3 phoA E15 (argF-lac)169 degP ompT kan^{r}; E. coli W3 110 strain 37D6, which has the complete genotype tonA ptr3 phoA E15 (argF-lac)169 degP ompT rbs7 ilvG kan^{r}; E. coli W3110 strain 40B4, which is strain 37D6 with a non-kanamycin resistant degP deletion mutation; and an E. coli strain having mutant periplasmic protease disclosed in U.S. Pat. No. 4,946,783 issued 7 Aug. 1990. Alternatively, in vitro methods of cloning, e.g., PCR or other nucleic acid polymerase reactions, are suitable.

Full length antibody, antibody fragments, and antibody fusion proteins can be produced in bacteria, in particular when glycosylation and Fc effector function are not needed, such as when the therapeutic antibody is conjugated to a cytotoxic agent (e.g., a toxin) and the immunoconjugate by itself shows effectiveness in tumor cell destruction. Full length antibodies have greater half life in circulation. Production in E. coli is faster and more cost efficient. For expression of antibody fragments and polypeptides in bacteria, see, e.g., U.S. Pat. No. 5,648,237 (Carter et. al.), U.S. Pat. No. 5,789,199 (Joly et al.), and U.S. Pat. No. 5,840,523 (Simmons et al.) which describes translation initiation region (TIR) and signal sequences for optimizing expression and secretion, these patents incorporated herein by reference. After expression, the antibody is isolated from the E. coli cell paste in a soluble fraction and can be purified through, e.g., a protein A or G column depending on the isotype. Final purification can be carried out similar to the process for purifying antibody expressed in suitable cells (e.g., CHO cells).

In addition to prokaryotes, eukaryotic microbes such as filamentous fungi or yeast are suitable cloning or expression hosts for vectors encoding GDM polypeptides. Saccharomyces cerevisiae is a commonly used lower eukaryotic host microorganism. Others include Schizosaccharomyces pombe (Beach and Nurse, Nature, 290: 140 [1981]; EP 139,383 published 2 May 1985); Kluyveromyces hosts (U.S. Pat. No. 4,943,529; Fleer et al., Bio/Technology, 9:968-975 (1991)) such as, e.g., K. lactis (MW98-8C, CBS683, CBS4574; Louvencourt et al., J. Bacteriol., 154(2):737-742 [1983]), K. fragilis (ATCC 12,424), K. bulgaricus (ATCC 16,045), K. wickeramii (ATCC 24,178), K. waltii (ATCC 56,500), K. drosophilarum (ATCC 36,906; Van den Berg et al., Bio/Technology, 8:135 (1990)), K. thermotolerans, and K. marxianus; yarrowia (EP 402,226); Pichia pastoris (EP 183,070; Sreekrishna et al., J. Basic Microbiol., 28:265-278 [1988]); Candida; Trichoderma reesia (EP 244,234); Neurospora crassa (Case et al., Proc. Natl. Acad. Sci. USA, 76:5259-5263 [1979]); Schwanniomyces such as Schwanniomyces occidentalis (EP 394,538 published 31 Oct. 1990); and filamentous fungi such as, e.g., Neurospora, Penicillium, Tolypocladium (WO 91/00357 published 10 Jan. 1991), and Aspergillus hosts such as A. nidulans (Ballance et al., Biochem. Biophys. Res. Commun., 112:284-289 [1983]; Tilburn et al., Gene, 26:205-221 [1983]; Yelton et al., Proc. Natl. Acad. Sci. USA, 81: 1470-1474 [1984]) and A. niger (Kelly and Hynes, EMBO J., 4:475-479 [1985]). Methylotropic yeasts are suitable herein and include, but are not limited to, yeast capable of growth on methanol selected from the genera consisting of Hansenula, Candida, Kloeckera, Pichia, Saccharomyces, Torulopsis, and Rhodotorula. A list of specific species that are exemplary of this class of yeasts may be found in C. Anthony, The Biochemistrv of Methylotrophs, 269 (1982).

Suitable host cells for the expression of glycosylated GDM polypeptide production are derived from multicellular organisms. Examples of invertebrate cells include insect cells such as Drosophila S2 and Spodoptera Sf9, as well as plant cells, such as cell cultures of cotton, corn, potato, soybean, petunia, tomato, and tobacco. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruitfly), and Bombyx mori have been identified. A variety of viral strains for transfection are publicly available, e.g., the L-1 variant of Autographa californica NPV and the Bm-5 strain of Bombyx mori NPV, and such viruses may be used as the virus herein according to the present invention, particularly for transfection of Spodoptera frugiperda cells.

However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

Host cells are transformed with the above-described expression or cloning vectors for GDM polypeptide production and cultured in conventional nutrient media modified as appropriate for inducing promoters, selecting transformants, or amplifying the genes encoding the desired sequences.

3. Selection and Use of a Replicable Vector

The nucleic acid (e.g., cDNA or genomic DNA) encoding the respective GDM polypeptide may be inserted into a replicable vector for cloning (amplification of the DNA) or for expression. Various vectors are publicly available. The vector may, for example, be in the form of a plasmid, cosmid, viral particle, or phage. The appropriate nucleic acid sequence may be inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan.

The GDM polypeptide may be produced recombinantly not only directly, but also as a fusion polypeptide with a heterologous polypeptide, which may be a signal sequence or other polypeptide having a specific cleavage site at the N-terminus of the mature protein or polypeptide. In general, the signal sequence may be a component of the vector, or it may be a part of the DNA encoding the mature sequence that is inserted into the vector. The signal sequence may be a prokaryotic signal sequence selected, for example, from the group of the alkaline phosphatase, penicillinase, lpp, or heat-stable enterotoxin TI leaders. For yeast secretion the signal sequence may be, e.g., the yeast invertase leader, alpha factor leader (including Saccharomyces and Kluyveromyces α-factor leaders, the latter described in U.S. Pat. No. 5,010,182), or acid phosphatase leader, the C. albicans glucoamylase leader (EP 362,179 published 4 Apr. 1990), or the signal described in WO 90/13646 published 15 Nov. 1990. In mammalian cell expression, mammalian signal sequences may be used to direct secretion of the protein, such as signal sequences from secreted polypeptides of the same or related species, as well as viral secretory leaders.

Both expression and cloning vectors contain a nucleic acid sequence that enables the vector to replicate in one or more selected host cells. Such sequences are well known for a variety of bacteria, yeast, and viruses. The origin of replication from the plasmid pBR322 is suitable for most Gram-negative bacteria, the 2μp plasmid origin is suitable for yeast, and various viral origins (SV40, polyoma, adenovirus, VSV or BPV) are useful for cloning vectors in mammalian cells.

Expression and cloning vectors will typically contain a selection gene, also termed a selectable marker. Typical selection genes encode proteins that (a) confer resistance to antibiotics or other toxins, e.g., ampicillin, neomycin, methotrexate, or tetracycline, (b) complement auxotrophic deficiencies, or (c) supply critical nutrients not available from complex media, e.g., the gene encoding D-alanine racemase for Bacilli.

An example of suitable selectable markers for mammalian cells are those that enable the identification of cells competent to take up nucleic acid encoding the desire protein, such as DHFR or thymidine kinase. An appropriate host cell when wild-type DHFR is employed is the CHO cell line deficient in DHFR activity, prepared and propagated as described by Urlaub et al., Proc. Natl. Acad. Sci. USA, 77:4216 (1980). A suitable selection gene for use in yeast is the trp1 gene present in the yeast plasmid YRp7 [Stinchcomb et al., Nature, 282:39 (1979); Kingsman et al., Gene, 7:141 (1979); Tschemper et al., Gene, 10:157 (1980)]. The trp1 gene provides a selection marker for a mutant strain of yeast lacking the ability to grow in tryptophan, for example, ATCC No. 44076 or PEP4-1 [Jones, Genetics, 85:12 (1977)].

Expression and cloning vectors usually contain a promoter operably linked to the nucleic acid sequence encoding the desired amino acid sequence, in order to direct mRNA synthesis. Promoters recognized by a variety of potential host cells are well known. Promoters suitable for use with prokaryotic hosts include the β-lactamase and lactose promoter systems [Chang et al., Nature, 275:615 (1978); Goeddel et al., Nature, 281:544 (1979)], alkaline phosphatase, a tryptophan (trp) promoter system [Goeddel, Nucleic Acids Res., 8:4057 (1980); EP 36,776], and hybrid promoters such as the tac promoter [deBoer et al., Proc. Natl. Acad. Sci. USA, 80:21-25 (1983)]. Promoters for use in bacterial systems also will contain a Shine-Dalgarno (S.D.) sequence operably linked to the DNA encoding the desired protein sequence.

Examples of suitable promoting sequences for use with yeast hosts include the promoters for 3-phosphoglycerate kinase [Hitzeman et al., J. Biol. Chem., 255:2073 (1980)] or other glycolytic enzymes [Hess et al., J. Adv. Enzyme Reg., 7:149 (1968); Holland, Biochemistrv, 17:4900 (1978)], such as enolase, glyceraldehyde-3-phosphate dehydrogenase, hexokinase, pyruvate decarboxylase, phosphofructokinase, glucose-6-phosphate isomerase, 3-phosphoglycerate mutase, pyruvate kinase, triosephosphate isomerase, phosphoglucose isomerase, and glucokinase.

Other yeast promoters, which are inducible promoters having the additional advantage of transcription controlled by growth conditions, are the promoter regions for alcohol dehydrogenase 2, isocytochrome C, acid phosphatase, degradative enzymes associated with nitrogen metabolism, metallothionein, glyceraldehyde-3-phosphate dehydrogenase, and enzymes responsible for maltose and galactose utilization. Suitable vectors and promoters for use in yeast expression are further described in EP 73,657.

DNA Transcription in mammalian host cells is controlled, for example, by promoters obtained from the genomes of viruses such as polyoma virus, fowlpox virus (UK 2,211,504 published 5 Jul. 1989), adenovirus (such as Adenovirus 2), bovine papilloma virus, avian sarcoma virus, cytomegalovirus, a retrovirus, hepatitis-B virus and Simian Virus 40 (SV40), from heterologous mammalian promoters, e.g., the actin promoter or an immunoglobulin promoter, and from heat-shock promoters, provided such promoters are compatible with the host cell systems.

Transcription of a DNA encoding the GDM polypeptide may be increased by inserting an enhancer sequence into the vector. Enhancers are cis-acting elements of DNA, usually about from 10 to 300 bp, that act on a promoter to increase its transcription. Many enhancer sequences are now known from mammalian genes (globin, elastase, albumin, α-fetoprotein, and insulin). Typically, however, one will use an enhancer from a eukaryotic cell virus. Examples include the SV40 enhancer on the late side of the replication origin (bp 100-270), the cytomegalovirus early promoter enhancer, the polyoma enhancer on the late side of the replication origin, and adenovirus enhancers. The enhancer may be spliced into the vector at a position 5′ or 3′ to the coding sequence of the preceding amino acid sequences, but is preferably located at a site 5′ from the promoter.

Expression vectors used in eukaryotic host cells (yeast, fungi, insect, plant, animal., human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. These regions contain nucleotide segments transcribed as polyadenylated fragments in the untranslated portion of the mRNA encoding the respective antibody, polypeptide or oligopeptide described in this section.

Still other methods, vectors, and host cells suitable for adaptation to the synthesis of the respective antibody, polypeptide or oligopeptide in recombinant vertebrate cell culture are described in Gething et al., Nature, 293:620-625 (1981); Mantei et al., Nature, 281:40-46 (1979); EP 117,060; and EP 117,058.

4. Culturing the Host Cells

The host cells used to produce the GDM polypeptides may be cultured in a variety of media. Commercially available media such as Ham's F10 (Sigma), Minimal Essential Medium ((MEM), (Sigma), RPMI-1640 (Sigma), and Dulbecco's Modified Eagle's Medium ((DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Patent Re. 30,985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to those skilled in the art. The culture conditions, such as temperature, pH, and the like, are those previously used with the host cell selected for expression, and will be apparent to the ordinarily skilled artisan.

5. Detecting Gene Amplification/Expression

Gene amplification and/or expression may be measured in a sample directly, for example, by conventional Southern blotting, Northern blotting to quantitate the transcription of mRNA [Thomas, Proc. Natl. Acad. Sci. USA, 77:5201-5205 (1980)], dot blotting (DNA analysis), or in situ hybridization, using an appropriately labeled probe, based on the sequences provided herein. Alternatively, antibodies may be employed that can recognize specific duplexes, including DNA duplexes, RNA duplexes, and DNA-RNA hybrid duplexes or DNA-protein duplexes. The antibodies in turn may be labeled and the assay may be carried out where the duplex is bound to a surface, so that upon the formation of duplex on the surface, the presence of antibody bound to the duplex can be detected.

Gene expression, alternatively, may be measured by immunological methods, such as immunohistochemical staining of cells or tissue sections and assay of cell culture or body fluids, to quantitate directly the expression of gene product. Antibodies useful for immunohistochemical staining and/or assay of sample fluids may be either monoclonal or polyclonal, and may be prepared in any mammal. Conveniently, the antibodies suitable for the present method may be prepared against a native sequence polypeptide or oligopeptide, or against exogenous sequence fused to DNA and encoding a specific antibody epitope of such a polypeptide or oligopeptide.

6. Protein Purification

GDM polypeptides may be recovered from culture medium or from host cell lysates. If membrane-bound, it can be released from the membrane using a suitable detergent solution (e.g. Triton-X 100) or by enzymatic cleavage. Cells employed in expression of the preceding can be disrupted by various physical or chemical means, such as freeze-thaw cycling, sonication, mechanical disruption, or cell lysing agents.

It may be desireable to purify the preceding from recombinant cell proteins or polypeptides. The following procedures are exemplary of suitable purification procedures: by fractionation on an ion-exchange column; ethanol precipitation; reverse phase HPLC; chromatography on silica or on a cation-exchange resin such as DEAE; chromatofocusing; SDS-PAGE; ammonium sulfate precipitation; gel filtration using, for example, Sephadex G-75; protein A Sepharose columns to remove contaminants such as IgG; and metal chelating columns to bind epitope-tagged forms of the desired molecules. Various methods of protein purification may be employed and such methods are known in the art and described for example in Deutscher, Methods in Enzymology, 182 (1990); Scopes, Protein Purification: Principles and Practice, Springer-Verlag, New York (1982). The purification step(s) selected will depend, for example, on the nature of the production process used and the particular antibody, polypeptide or oligopeptide produced for the claimed methods.

When using recombinant techniques, the GDM polypeptide can be produced intracellularly, in the periplasmic space, or directly secreted into the medium. If such molecules are produced intracellularly, as a first step, the particulate debris, either host cells or lysed fragments, are removed, for example, by centrifugation or ultrafiltration. Carter et al., Bio/Technology 10:163-167 (1992) describe a procedure for isolating antibodies which are secreted to the periplasmic space of E. coli. Briefly, cell paste is thawed in the presence of sodium acetate (pH 3.5), EDTA, and phenylmethylsulfonylfluoride (PMSF) over about 30 min. Cell debris can be removed by centrifugation. Where the antibody is secreted into the medium, supernatants from such expression systems are generally first concentrated using a commercially available protein concentration filter, for example, an Amicon or Millipore Pellicon ultrafiltration unit. A protease inhibitor such as PMSF may be included in any of the foregoing steps to inhibit proteolysis and antibiotics may be included to prevent the growth of adventitious contaminants.

Purification can occur using, for example, hydroxylapatite chromatography, gel electrophoresis, dialysis, and affinity chromatography, with affinity chromatography being the preferred purification technique. The suitability of protein A as an affinity ligand depends on the species and isotype of any immunoglobulin Fc domain that is present in the antibody. Protein A can be used to purify antibodies that are based on human γ1, γ2 or γ4 heavy chains (Lindmark et al., J. Immunol. Meth. 62:1-13 (1983)). Protein G is recommended for all mouse isotypes and for human γ3 (Guss et al., EMBO J. 5:15671575 (1986)). The matrix to which the affinity ligand is attached is most often agarose, but other matrices are available. Mechanically stable matrices such as controlled pore glass or poly(styrenedivinyl)benzene allow for faster flow rates and shorter processing times than can be achieved with agarose. Where the antibody comprises a C_{H}3 domain, the Bakerbond ABX™resin (J. T. Baker, Phillipsburg, N.J.) is useful for purification. Other techniques for protein purification such as fractionation on an ion-exchange column, ethanol precipitation, Reverse Phase HPLC, chromatography on silica, chromatography on heparin SEPHAROSE™ chromatography on an anion or cation exchange resin (such as a polyaspartic acid column), chromatofocusing, SDS-PAGE, and ammonium sulfate precipitation are also available depending on the antibody to be recovered.

Following any preliminary purification step(s), the mixture comprising the antibody of interest and contaminants may be subjected to low pH hydrophobic interaction chromatography using an elution buffer at a pH between about 2.5-4.5, preferably performed at low salt concentrations (e.g., from about 0-0.25 M salt).

I. Pharmaceutical Formulations

Therapeutic formulations of the GDM antagonists (“therapeutic agent”) used in accordance with the present invention may be prepared for storage by mixing the therapeutic agent(s) having the desired degree of purity with optional pharmaceutically acceptable carriers, excipients or stabilizers (Remington: The Science of Practice of Pharmacy, 20th edition, Gennaro, A. et al., Ed., Philadelphia College of Pharmacy and Science (2000)), in the form of lyophilized formulations or aqueous solutions. Acceptable carriers, excipients, or stabilizers are nontoxic to recipients at the dosages and concentrations employed, and include buffers such as acetate, Tris, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; tonicifiers such as trehalose and sodium chloride; sugars such as sucrose, mannitol, trehalose or sorbitol; surfactant such as polysorbate; salt-forming counter-ions such as sodium; metal complexes (e.g., Zn-protein complexes); and/or non-ionic surfactants such as TWLEEN®, PLURONICS® or polyethylene glycol (PEG). The antibody preferably comprises the antibody at a concentration of between 5-200 mg/ml, preferably between 10-100 mg/ml.

The formulations herein may also contain more than one active compound as necessary for the particular indication being treated, preferably those with complementary activities that do not adversely affect each other. For example, in addition to the preceding therapeutic agent(s), it may be desirable to include in the formulation, an additional antibody, e.g., a second such therapeutic agent, or an antibody to some other target such as a growth factor that affects the growth of the glioma. Alternatively, or additionally, the composition may further comprise a chemotherapeutic agent, cytotoxic agent, cytokine, growth inhibitory agent, anti-hormonal agent, and/or cardioprotectant. Such molecules are suitably present in combination in amounts that are effective for the purpose intended.

The active ingredients may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy, supra.

Sustained-release preparations may be prepared. Suitable examples of sustained-release preparations include semi-permeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(-)-3-hydroxybutyric acid.

The formulations to be used for in vivo administration must be sterile. This is readily accomplished by filtration through sterile filtration membranes.

J. Diagnosis and Treatment with GDM Antagonists

To determine GDM expression in the glioma, various diagnostic assays are available. In one embodiment, GDM polypeptide overexpression may be analyzed by immunohistochemistry (IHC). Parrafin embedded tissue sections from a tumor biopsy may be subjected to the IHC assay and accorded a GDM protein staining intensity criteria as follows:

Score 0—no staining is observed or membrane staining is observed in less than 10% of tumor cells.

Score 1+—a faint/barely perceptible membrane staining is detected in more than 10% of the tumor cells. The cells are only stained in part of their membrane.

Score 2+—a weak to moderate complete membrane staining is observed in more than 10% of the tumor cells.

Score 3+—a moderate to strong complete membrane staining is observed in more than 10% of the tumor cells.

Those tumors with 0 or 1+ scores for GDM polypeptide expression may be characterized as not overexpressing GDM, whereas those tumors with 2+or 3+ scores may be characterized as overexpressing GDM.

Alternatively, or additionally, FISH assays such as the INFORM® (sold by Ventana, Arizona) or PATHVISION® (Vysis, Illinois) may be carried out on formalin-fixed, paraffin-embedded tumor tissue to determine the extent (if any) of GDM overexpression in the tumor.

GDM overexpression or amplification may be evaluated using an in vivo diagnostic assay, e.g., by administering a molecule (such as an antibody, oligopeptide or organic molecule) which binds the molecule to be detected and is tagged with a detectable label (e.g., a radioactive isotope or a fluorescent label) and externally scanning the patient for localization of the label.

Currently, depending on the stage of the cancer, cancer treatment involves one or a combination of the following therapies: surgery to remove the cancerous tissue, radiation therapy, and chemotherapy. Therapy comprising of administering GDM antagonists may be especially desirable in elderly patients who do not tolerate the toxicity and side effects of chemotherapy well and in metastatic disease where radiation therapy has limited usefulness. The tumor targeting GDM antagonists of the present inventive method may also be used to alleviate GDM-expressing cancers upon initial diagnosis of the disease or during relapse. For therapeutic applications, such GDM antagonists can be used in combination with, before or after application of other conventional agents and/or methods for the treatment of glioma, e.g., hormones, antiangiogens, or radiolabelled compounds, or with surgery, cryotherapy, radiotherapy and/or chemotherapy. Chemotherapeutic drugs such as TAXOTERE® (docetaxel), TAXOL® (palictaxel), estramustine and mitoxantrone are used in treating cancer, in particular, in good risk patients.

In particular, combination therapy with palictaxel and modified derivatives (see, e.g., EP0600517) is contemplated. The preceding antibody, polyeptpide, oligopeptide or organic molecule will be administered with a therapeutically effective dose of the chemotherapeutic agent. In another embodiment, such antibody, polypeptide, oligopeptide or organic molecule is administered in conjunction with chemotherapy to enhance the activity and efficacy of the chemotherapeutic agent, e.g., paclitaxel. The Physicians' Desk Reference (PDR) discloses dosages of these agents that have been used in treatment of various cancers. The dosing regimen and dosages of these aforementioned chemotherapeutic drugs that are therapeutically effective will depend on the particular cancer being treated, the extent of the disease and other factors familiar to the physician of skill in the art and can be determined by the physician.

In one particular embodiment, an immunoconjugate comprising such a GDM antagonist conjugated with a cytotoxic agent is administered to the patient. Preferably, such immunoconjugate is internalized by the cell, resulting in increased therapeutic efficacy of the immunoconjugate in killing the cancer cell to which it binds. In a preferred embodiment, the cytotoxic agent targets or interferes with the nucleic acid in the cancer cell. Examples of such cytotoxic agents are described above and include maytansinoids, calicheamicins, ribonucleases and DNA endonucleases.

The preceding GDM antagonists or toxin conjugates thereof are administered to a human patient, in accord with known methods, such as intravenous administration, e.g., as a bolus or by continuous infusion over a period of time, by intracranial, intracerobrospinal, intra-articular, intrathecal, intravenous, intraarterial, subcutaneous, oral, topical, or inhalation routes.

Other therapeutic regimens may be combined with the administration of the foregoing GDM antagonists. The combined administration includes co-administration, using separate formulations or a single pharmaceutical formulation, and consecutive administration in either order, wherein preferably there is a time period while both (or all) active agents simultaneously exert their biological activities. Preferably such combined therapy results in a synergistic therapeutic effect.

In another embodiment, the therapeutic treatment methods of the present invention involves the combined administration of the above GDM antagonist and one or more chemotherapeutic agents or growth inhibitory agents, including co-administration of cocktails of different chemotherapeutic agents. Example chemotherapeutic agents have been provided previously. Preparation and dosing schedules for such chemotherapeutic agents may be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

For the prevention or treatment of disease, the dosage and mode of administration will be chosen by the physician according to known criteria. The appropriate dosage of GDM antagonists will depend on the type of disease to be treated, the severity and course of the disease, whether administration is for preventive or therapeutic purposes, previous therapy (including) the patient's clinical history and response, and the discretion of the attending physician. The preceding GDM antagonists may be suitably administered to the patient at one time or over a series of treatments. Administration may occur by intravenous infusion or by subcutaneous injections. Depending on the type and severity of the disease, about 1 μg/kg to about 50 mg/kg body weight (e.g., about 0.1-15 mg/kg/dose) of GDM antagonist can be an initial candidate dosage for administration to the patient, whether, for example, by one or more separate administrations, or by continuous infusion. A dosing regimen can comprise administering an initial loading dose of about 4 mg/kg, followed by a weekly maintenance dose of about 2 mg/kg of such a GDM antagonist. However, other dosage regimens may be useful. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. The progress of this therapy can be readily monitored by conventional methods and assays and based on criteria known to the physician or other persons of skill in the art.

Aside from administration of the antibody protein to the patient, the present application contemplates administration of the antibody by gene therapy. Such administration of nucleic acid encoding the GDM polypeptide antagonists is encompassed by the expression “administering a therapeutically effective amount of an antibody”. See, for example, WO 96/07321 published Mar. 14, 1996 concerning the use of gene therapy to generate intracellular antibodies.

There are two major approaches to getting such nucleic acid (optionally contained in a vector) into the patient's cells; in vivo and ex vivo. For in vivo delivery the nucleic acid is injected directly into the patient, usually at the site where the antibody is required. For ex vivo treatment, the patient's cells are removed, the nucleic acid is introduced into these isolated cells and the modified cells are administered to the patient either directly or, for example, encapsulated within porous membranes which are implanted into the patient (see, e.g., U.S. Pat. Nos. 4,892,538 and 5,283,187). There are a variety of techniques available for introducing nucleic acids into viable cells. The techniques vary depending upon whether the nucleic acid is transferred into cultured cells in vitro, or in vivo in the cells of the intended host. Techniques suitable for the transfer of nucleic acid into mammalian cells in vitro include the use of liposomes, electroporation, microinjection, cell fusion, DEAE-dextran, the calcium phosphate precipitation method, etc. A commonly used vector for ex vivo delivery of the gene is a retroviral vector.

The currently preferred in vivo nucleic acid transfer techniques include transfection with viral vectors (such as adenovirus, Herpes simplex I virus, or adeno-associated virus) and lipid-based systems (useful lipids for lipid-mediated transfer of the gene are DOTMA, DOPE and DC-Chol, for example). For review of the currently known gene marking and gene therapy protocols see Anderson et al., Science 256:808-813 (1992). See also WO 93/25673 and the references cited therein.

K. Articles of Manufacture and Kits

For therapeutic applications, the article of manufacture comprises a container and a label or package insert on or associated with the container indicating a use for the treatment of glioma. Suitable containers include, for example, bottles, vials, syringes, etc. The containers may be formed from a variety of materials such as glass or plastic. The container holds a composition which is effective for treating the cancer condition and may have a sterile access port (for example the container may be an intravenous solution bag or a vial having a stopper pierceable by a hypodermic injection needle). At least one active agent in the composition is a GDM antagonist. The label or package insert indicates that the composition is used for treating glioma. The label or package insert will further comprise instructions for administering the GDM antagonist. Additionally, the article of manufacture may further comprise a second container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Kits may also be provided that are useful for various other purposes, e.g., for GDM-expressing cell killing assays, for purification or immunoprecipitation of GDM polypeptide from cells. For isolation and purification of GDM polypeptide, the kit can contain the respective GDM-binding reagent coupled to beads (e.g., sepharose beads). Kits can be provided which contain such molecules for detection and quantitation of GDM polypeptide in vitro, e.g., in an ELISA or a Western blot. As with the article of manufacture, the kit comprises a container and a label or package insert on or associated with the container. The container holds a composition comprising at least one such GDM binding-antibody, oligopeptide or organic molecule useable with the invention. Additional containers may be included that contain, e.g., diluents and buffers, control antibodies. The label or package insert may provide a description of the composition as well as instructions for the intended in vitro or diagnostic use.

L. Sense and Anti-sense GDM-Encoding Nucleic Acids

GDM antagonists include fragments of the GDM-encoding nucleic acids such as antisense or and oligonucleotides, which comprise a single-stranded nucleic acid sequence (either RNA or DNA) capable of binding to target GDM mRNA (sense) or GDM DNA (antisense) sequences. Antisense or sense oligonucleotides, according to the present invention, comprises a fragment of the coding region of the respective GDM DNA. The ability to derive an antisense or a sense oligonucleotide, based upon a cDNA sequence encoding a given protein is described in, for example, Stein and Cohen (Cancer Res. 48:2659, 1988) and van der Krol et al. (BioTechniques 6:958, 1988).

Binding of antisense or sense oligonucleotides to target nucleic acid sequences results in the formation of duplexes that block transcription or translation of the target sequence by one of several means, including enhanced degradation of the duplexes, premature termination of transcription or translation, or by other means. Such methods are encompassed by the present invention. The antisense oligonucleotides thus may be used to block expression of GDM proteins, wherein those GDM proteins may play a role in the induction of cancer in mammals. Antisense or sense oligonucleotides further comprise oligonucleotides having modified sugar-phosphodiester backbones (or other sugar linkages, such as those described in WO 91/06629) and wherein such sugar linkages are resistant to endogenous nucleases. Such oligonucleotides with resistant sugar linkages are stable in vivo (i.e., capable of resisting enzymatic degradation) but retain sequence specificity to be able to bind to target nucleotide sequences.

The antisense compounds used in accordance with this invention may be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives. The compounds of the invention may also be admixed, encapsulated, conjugated or otherwise associated with other molecules, molecule structures or mixtures of compounds, as for example, liposomes, receptor targeted molecules, oral, rectal, topical or other formulations, for assisting in uptake, distribution and/or absorption. Patents that teach the preparation of such uptake, distribution and/or absorption assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756, each of which is herein incorporated by reference.

Other examples of sense or antisense oligonucleotides include those oligonucleotides which are covalently linked to organic moieties, such as those described in WO 90/10048, and other moieties that increases affinity of the oligonucleotide for a target nucleic acid sequence, such as poly-(L-lysine). Further still, intercalating agents, such as ellipticine, and alkylating agents or metal complexes may be attached to sense or antisense oligonucleotides to modify binding specificities of the antisense or sense oligonucleotide for the target nucleotide sequence.

Antisense or sense oligonucleotides may be introduced into a cell containing the target nucleic acid sequence by any gene transfer method, including, for example, CaPO₄-mediated DNA transfection, electroporation, or by using gene transfer vectors such as Epstein-Barr virus. In a preferred procedure, an antisense or sense oligonucleotide is inserted into a suitable retroviral vector. A cell containing the target nucleic acid sequence is contacted with the recombinant retroviral vector, either in vivo or ex vivo. Suitable retroviral vectors include, but are not limited to, those derived from the murine retrovirus M-MuLV, N2 (a retrovirus derived from M-MuLV), or the double copy vectors designated DCT5A, DCT5B and DCT5C (see WO 90/13641).

Sense or antisense oligonucleotides also may be introduced into a cell containing the target nucleotide sequence by formation of a conjugate with a ligand binding molecule, as described in WO 91/04753. Suitable ligand binding molecules include, but are not limited to, cell surface receptors, growth factors, other cytokines, or other ligands that bind to cell surface receptors. Preferably, conjugation of the ligand binding molecule does not substantially interfere with the ability of the ligand binding molecule to bind to its corresponding molecule or receptor, or block entry of the sense or antisense oligonucleotide or its conjugated version into the cell.

Alternatively, a sense or an antisense oligonucleotide may be introduced into a cell containing the target nucleic acid sequence by formation of an oligonucleotide-lipid complex, as described in WO 90/10448. The sense or antisense oligonucleotide-lipid complex is preferably dissociated within the cell by an endogenous lipase.

Antisense or sense RNA or DNA molecules are generally at least about 5 nucleotides in length, alternatively at least about 6, 7, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, or 1000 nucleotides in length, wherein in this context the term “about” means the referenced nucleotide sequence length plus or minus 10% of that referenced length.

M. Screening Assays for Use in Identification of GDM Antagonists:

The assays can be performed in a variety of formats, including protein-protein binding assays, biochemical screening assays, immunoassays, and cell-based assays, which are well characterized in the art.

All assays for antagonists are common in that they call for contacting the drug candidate with a GDM polypeptide encoded by a nucleic acid identified herein under conditions and for a time sufficient to allow these two components to interact.

In binding assays, the interaction is binding and the complex formed can be isolated or detected in the reaction mixture. In a particular embodiment, the GDM polypeptide encoded by the gene identified herein or the drug candidate is immobilized on a solid phase, e.g., on a microtiter plate, by covalent or non-covalent attachments. Non-covalent attachment generally is accomplished by coating the solid surface with a solution of the GDM polypeptide and drying. Alternatively, an immobilized antibody, e.g., a monoclonal antibody, specific for the GDM polypeptide to be immobilized can be used to anchor it to a solid surface. The assay is performed by adding the non-immobilized component, which may be labeled by a detectable label, to the immobilized component, e.g., the coated surface containing the anchored component. When the reaction is complete, the non-reacted components are removed, e.g., by washing, and complexes anchored on the solid surface are detected. When the originally non-immobilized component carries a detectable label, the detection of label immobilized on the surface indicates that complexing occurred. Where the originally non-immobilized component does not carry a label, complexing can be detected, for example, by using a labeled antibody specifically binding the immobilized complex.

If the candidate compound interacts with but does not bind to a particular GDM polypeptide encoded by a gene identified herein, its interaction with that polypeptide can be assayed by methods well known for detecting protein-protein interactions. Such assays include traditional approaches, such as, e.g., cross-linking, co-immunoprecipitation, and co-purification through gradients or chromatographic columns. In addition, protein-protein interactions can be monitored by using a yeast-based genetic system described by Fields and co-workers (Fields and Song, Nature (London), 340:245-246 (1989); Chien et al., Proc. Natl. Acad. Sci. USA, 88:9578-9582 (1991)) as disclosed by Chevray and Nathans, Proc. Natl. Acad. Sci. USA, 89: 5789-5793 (1991). Many transcriptional activators, such as yeast GAL4, consist of two physically discrete modular domains, one acting as the DNA-binding domain, the other one functioning as the transcription-activation domain. The yeast expression system described in the foregoing publications (generally referred to as the “two-hybrid system”) takes advantage of this property, and employs two hybrid proteins, one in which the target protein is fused to the DNA-binding domain of GAL4, and another, in which candidate activating proteins are fused to the activation domain. The expression of a GAL1-lacZ reporter gene under control of a GAL4-activated promoter depends on reconstitution of GAL4 activity via protein-protein interaction. Colonies containing interacting polypeptides are detected with a chromogenic substrate for β-galactosidase. A complete kit (MATCHMAKER™) for identifying protein-protein interactions between two specific proteins using the two-hybrid technique is commercially available from Clontech. This system can also be extended to map protein domains involved in specific protein interactions as well as to pinpoint amino acid residues that are crucial for these interactions.

Compounds that interfere with the interaction of a gene encoding a GDM polypeptide identified herein and other intra- or extracellular components can be tested as follows: usually a reaction mixture is prepared containing the product of the gene and the intra- or extracellular component under conditions and for a time allowing for the interaction and binding of the two products. To test the ability of a candidate compound to inhibit binding, the reaction is run in the absence and in the presence of the test compound. In addition, a placebo may be added to a third reaction mixture, to serve as positive control. The binding (complex formation) between the test compound and the intra- or extracellular component present in the mixture is monitored as described hereinabove. The formation of a complex in the control reaction(s) but not in the reaction mixture containing the test compound indicates that the test compound interferes with the interaction of the test compound and its reaction partner.

To assay for antagonists, the GDM polypeptide may be added to a cell along with the compound to be screened for a particular activity and the ability of the compound to inhibit the activity of interest in the presence of the GDM polypeptide indicates that the compound is an antagonist to the GDM polypeptide. Alternatively, antagonists may be detected by combining the GDM polypeptide and a potential antagonist with membrane-bound GDM polypeptide receptors or recombinant receptors under appropriate conditions for a competitive inhibition assay. The GDM polypeptide can be labeled, such as by radioactivity, such that the number of GDM polypeptide molecules bound to the receptor can be used to determine the effectiveness of the potential antagonist. The gene encoding the receptor can be identified by numerous methods known to those of skill in the art, for example, ligand panning and FACS sorting. Coligan et al., Current Protocols in Immun., 1(2): Chapter 5 (1991). Preferably, expression cloning is employed wherein polyadenylated RNA is prepared from a cell responsive to the GDM polypeptide and a cDNA library created from this RNA is divided into pools and used to transfect COS cells or other cells that are not responsive to the GDM polypeptide. Transfected cells that are grown on glass slides are exposed to labeled GDM polypeptide. The GDM polypeptide can be labeled by a variety of means including iodination or inclusion of a recognition site for a site-specific protein kinase. Following fixation and incubation, the slides are subjected to autoradiographic analysis. Positive pools are identified and sub-pools are prepared and re-transfected using an interactive sub-pooling and re-screening process, eventually yielding a single clone that encodes the putative receptor.

As an alternative approach for receptor identification, labeled GDM polypeptide can be photoaffinity-linked with cell membrane or extract preparations that express the receptor molecule. Cross-linked material is resolved by PAGE and exposed to X-ray film. The labeled complex containing the receptor can be excised, resolved into peptide fragments, and subjected to protein micro-sequencing. The amino acid sequence obtained from micro-sequencing would be used to design a set of degenerate oligonucleotide probes to screen a cDNA library to identify the gene encoding the putative receptor.

In another assay for antagonists, mammalian cells or a membrane preparation expressing the receptor would be incubated with labeled GDM polypeptide in the presence of the candidate compound. The ability of the compound to enhance or block this interaction could then be measured.

More specific examples of potential antagonists include an oligonucleotide that binds to the fusions of immunoglobulin with GDM polypeptide, and, in particular, antibodies including, without limitation, poly- and monoclonal antibodies and antibody fragments, single-chain antibodies, anti-idiotypic antibodies, and chimeric or humanized versions of such antibodies or fragments, as well as human antibodies and antibody fragments. Alternatively, a potential antagonist may be a closely related protein, for example, a mutated form of the GDM polypeptide that recognizes the receptor but imparts no effect, thereby competitively inhibiting the action of the GDM polypeptide.

Another potential GDM polypeptide antagonist is an antisense RNA or DNA construct prepared using antisense technology, where, e.g., an antisense RNA or DNA molecule acts to block directly the translation of mRNA by hybridizing to targeted mRNA and preventing protein translation.

Potential antagonists include small molecules that bind to the active site, the receptor binding site, or growth factor or other relevant binding site of the GDM polypeptide, thereby blocking the normal biological activity of the GDM polypeptide. Examples of small molecules include, but are not limited to, small peptides or peptide-like molecules, preferably soluble peptides, and synthetic non-peptidyl organic or inorganic compounds.

Ribozymes are enzymatic RNA molecules capable of catalyzing the specific cleavage of RNA. Ribozymes act by sequence-specific hybridization to the complementary target RNA, followed by endonucleolytic cleavage. Specific ribozyme cleavage sites within a potential RNA target can be identified by known techniques. For further details see, e.g., Rossi, Current Biology, 4:469-471 (1994), and PCT publication No. WO 97/33551 (published Sep. 18, 1997).

Nucleic acid molecules in triple-helix formation used to inhibit transcription should be single-stranded and composed of deoxynucleotides. The base composition of these oligonucleotides is designed such that it promotes triple-helix formation via Hoogsteen base-pairing rules, which generally require sizeable stretches of purines or pyrimidines on one strand of a duplex. For further details see, e.g., PCT publication No. WO 97/33551, supra.

These small molecules can be identified by any one or more of the screening assays discussed hereinabove and/or by any other screening techniques well known for those skilled in the art.

The following examples are offered for illustrative purposes only, and are not intended to limit the scope of the present invention in any way.

All patent and literature references cited in the present specification are hereby incorporated by reference in their entirety.

### EXAMPLES

### Example 1:

Experimental Procedures

Tumor Samples and Patient Characteristics

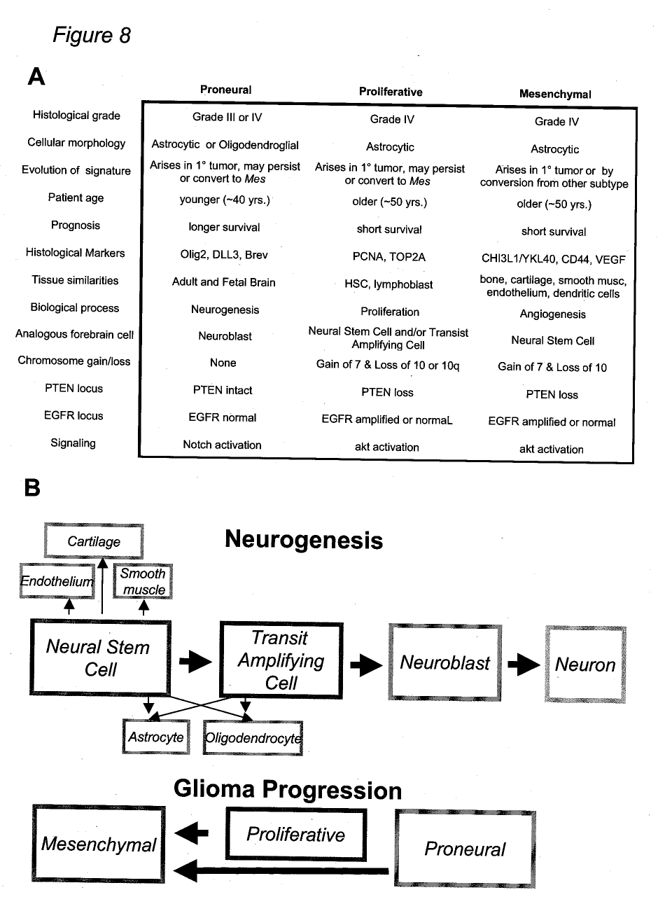

A summary of all tumor cases studied is included in FIG. 8A. For survival analysis, three expression profiling datasets were analyzed. We obtained frozen tissue samples from 76 cases at MDA, RNA from 39 cases from UCSF (Nigro et al., Cancer Res. 65: 1678-1686 (2005), and data from a previously published study from UCLA (Freije et al., supra.). Cases analyzed in the first two datasets met the following criteria: Fresh-frozen samples were obtained at the time of initial surgical resection from patients (>21 years of age) who did not receive prior radio- or chemotherapy. Clinical follow-up information was available for a period of at least 2 years post-surgery or until death. Institutional Review Board/Human Subjects approval was obtained for these retrospective laboratory studies at UCSF and MDA. Cases were graded as AA or GBM according to WHO criteria and sections from all tissues were examined by a neuropathologist (KA) to ensure that >90% of the sample represented tumor. Unusual histological variants were excluded. For the UCLA dataset, we utilized data from all cases in the previously published study that met our criteria for patient age and microarray quality control parameters. This dataset includes cases of oligodendroglial or mixed morphology as well as cases with survival times censored at less than 2 years.

Normal adult brain tissue consists of autopsy specimens of cerebral cortex from donors with no history of brain tumor or neurological disorders and was obtained from the National Neurological Rsearch Brain Bank (Los Angeles, Calif.).

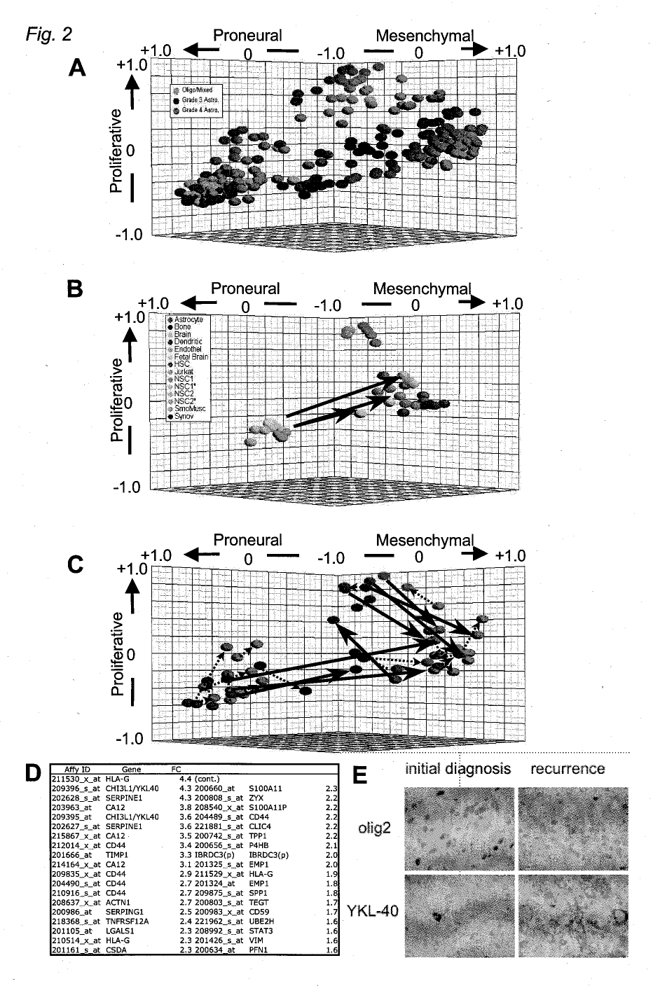

For comparisons of tumor grade vs. signature class (FIG. 2A), we include additional sample specimens for which clinical histories are not available. Normal adult brain tissue consists of autopsy specimens of cerebral cortex from with no history of brain tumor or neurological disorders and was obtained from the National Neurological Research Brain Bank (Los Angeles, Calif.). With the exception of specimens of brain and fetal astrocytes, data for samples analyzed in FIG. 2B were obtained via subscription to the Gene Logic (Gaithersburgh, Md.) database of Affymetrix data from human samples.

Gene Expression Profiling, Comparative Genomic Hybridization, and Quantitative PCR

For specimens not included in previous publications (Freije et al., supra; Nigro et al., supra) total cellular RNA from tumor and normal brain specimens was extracted using Qiagen's RNA isolation kit according to manufacturer's protocol. Genomic DNA contamination was removed through an on-column DNase digestion step. Affymetrix U133A & B chips were employed for expression profiling according to a previously published technique (Tumor Analysis Best Practices Working, 2004). Quality control parameters were as described in this publication with the exception that samples were allowed to display 3′/5′ ratios of >3 for actin provided that the 3′/5′ ratio for GAPDH was <3. Quantitative PCR was performed in duplicate for each sample on the ABI Prism7700 Sequence Detector (Applied Biosystems, Foster City, Calif.) with Taqman PCR Core reagents (Applied Biosystems, Foster City, Calif.). 25 ng of total RNA was used in each 50 μl reaction. Rab 14 was used for normalization as it exhibits very little variation in expression across a large number of samples. All primer pairs were designed to generate amplicons of 67-79 bp. DNA extraction and array CGH is performed as previously described. Misra et al., Clin. Cancer Res. 11(8): 2907-18 (2005). Of the 96 samples analyzed via CGH, data for 43 samples come from a previously published study (Nigro et al., supra.).

Analysis of Microarray Data

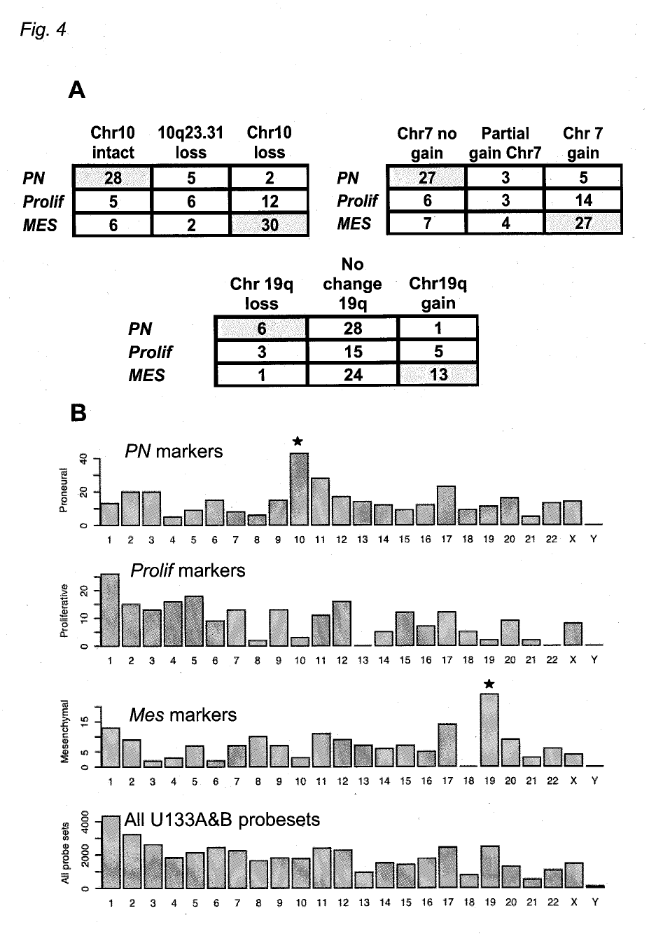

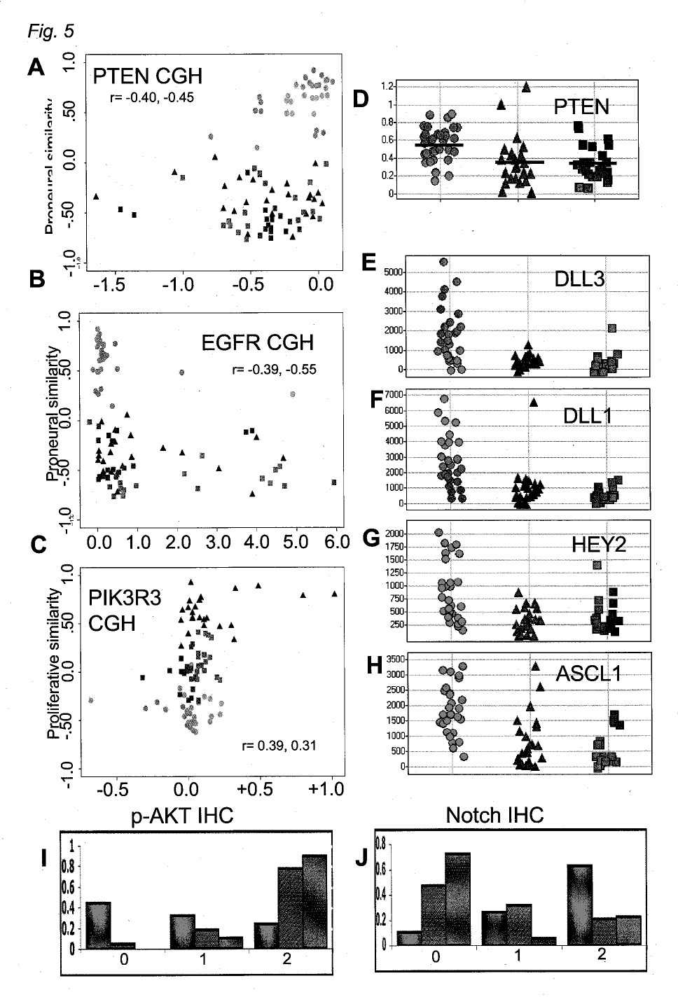

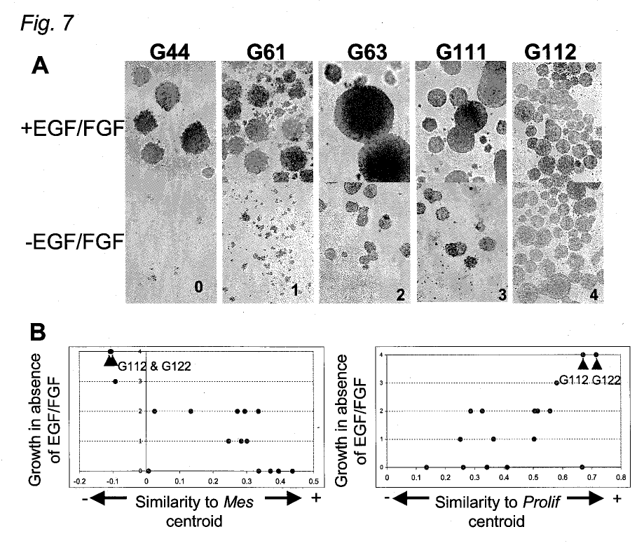

Signal intensity values from Microarray Analysis Suite version were utilized with a scaling factor of 500 for all analysis of microarray data. K-means clustering and agglomerative clustering were performed with the use of Spotfire Decision Site software version 7.3 using Pearson correlation as the similarity measure. For each gene, data was normalized into Z scores prior to clustering. The 35 signature genes used for tumor classification represent the most robust markers for each of three tumor subsets in the MDA survival sample set. Markers of each subset are identified as follows: Each of the 76 samples is assigned to one of three groups based on k-means clustering of expression of the 108 genes most strongly correlated with survival. Using a p value cutoff of 1×10⁻⁴, t tests were utilized to identify all genes whose expression differed between samples in each subclass compared to tumors of other subclasses (see Table A, GDM). For each comparison, we then rank-ordered the 500 probe sets with the smallest p values, by fold change. Probe sets used as signature genes for each tumor subtype were those corresponding to identified full-length sequences which showed the greatest fold change and which met a minimum expression cutoff (mean intensity of 400 within group of interest). Empirical determinations using k-means clustering revealed that stable sample clusters resulted when clustering was performed using 30-60 probe sets, provided that the gene list for clustering is balanced to include fewer markers of one of the three tumor subtypes. The final 35 signature genes contain 15 PN, 15 Mes, and 5 Prolif markers. Similarity scores to PN, Prolif and Mes centroids represent Pearson correlation coefficients to each of the centroids generated in k-means clustering of the 76 MDA survival samples. For statistical comparisons of data presented in FIG. 4 F-I and FIG. 7, log-transformed data was analyzed via t tests corrected for multiple group comparisons. Log-transformed expression data was also utilized for the statistical analysis performed for FIG. 5.

In situ Hybridization and Immunohistochemistry

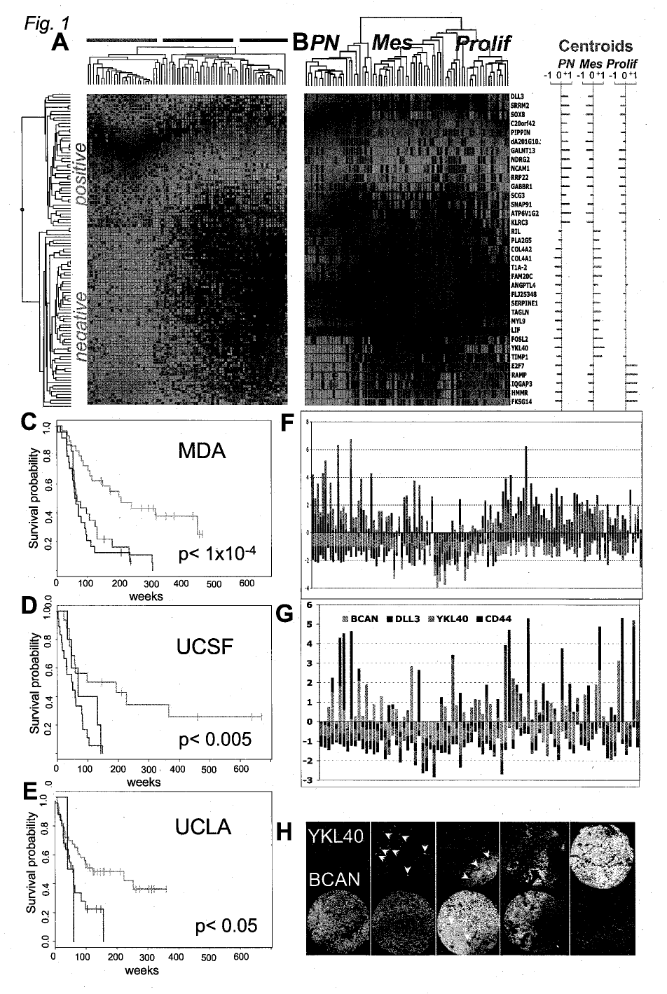

³³P-UTP-labeled antisense riboprobes were transcribed using Promega's in vitro transcription system (Promega, Madison, Wis.) and hybridized to paraffin-embedded human glioma tissue microarrays obtained from a variety of sources utilizing a previously-described method (Phillips et al., Science 250: 290-294 (1990). Images depicted in FIG. 1 are of a tissue cores included in a tissue microarray obtained from Petagen, Inc. Probe to BCAN represents a 668 bp fragment (939 to 1606) and for CHI3L1/YKL40 a 1158 bp fragment (121 to 1278).

Immunohistochemistry was performed on paraffin-embedded sections as previously described (Simmons et al., Cancer Res. 61: 1122-1128 (2001). Primary antibodies were anti-p-akt (ser473) from Cell Signaling Technology (Beverly, Mass.) and anti-Notch from Santa Cruz Biotechnology (Santa Cruz, Calif.). Ratings were performed by a neuropathologist blinded to the signature group of the specimens analyzed.

In vitro studies

Previously-described GBM cell lines are utilized for in vitro studies (Hartmann et al., Internat. J. Oncol. 15: 975-982 (1999). For neurosphere cultures, cells positively sorted with CD133 beads were maintained in culture as described for primary GBM specimens (Singh et al., Nature 432 396-401 (2004). All neurosphere cultures are maintained in Neurobasal medium (Invitrogen) with N2 supplement (Invitrogen) and NSF1 (Cambrex). When present, EGF and FGF are added at a concentration of 20 ng/ml. Each cell line is rated for neurosphere growth in the absence of EGF+FGF by an observer blinded to the molecular signature of the cell lines. Rating scale is as follows: 0=no viable neurospheres, 1=slowly expanding neurospheres, 2=moderate growth rate, 3=moderate to fast growth, but slower than that seen with EGF+FGF, 4=rapid growth that is not accelerated by EGF+FGF.

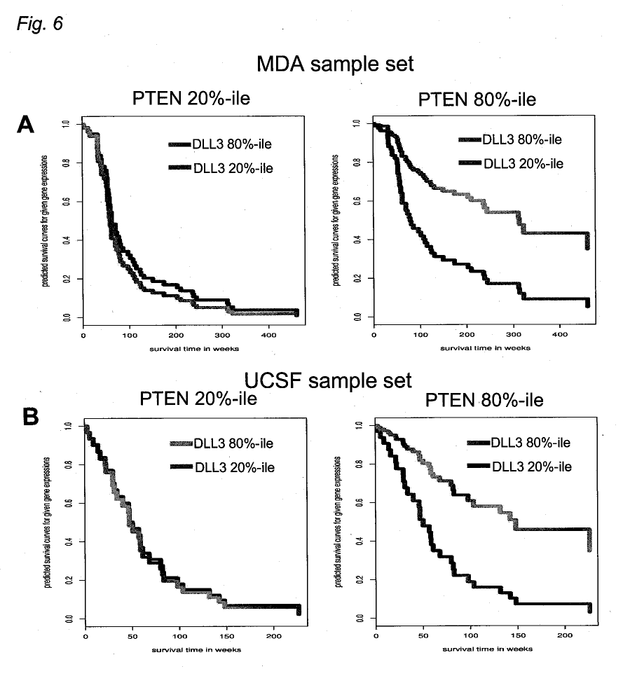

Growth inhibition assays are conducted in 96 well plates using standard cell culture conditions in DMEM medium with 10% fetal bovine serum. For treatment with rapamycin, LY294002, or the gamma secretase inhibitors GSI-1 or S-2188, cells were plated (Day-0) in ninety-six-well plate at a cell density of 1500 or 2000 per well, treated on Day-I with drug, and assessed for viability on Day-4 using Alamar Blue read-out. Each treatment condition was evaluated in a minimum of three experiments with the exception of S-2188 which yielded nearly identical results in two assays. Results shown in FIG. 6 are from one representative experiment.

Results

Molecular Signatures Define Prognostic Subclasses of High-grade Astrocytoma

76 samples from newly-diagnosed cases of GBM (n═55) and AA (n═21) were obtained from MD Anderson Cancer Center (MDA) and profiled via DNA microarrays to identify gene expression patterns that classify tumors into groups with differing prognoses (FIG. 1 A-C). Demographics for these and all tumor cases analyzed in the current study are described in FIG. 8A. We first identified probe sets whose expression most strongly correlates with survival (Spearman r of log-transformed expression intensity values vs. survival times >0.45 or <-0.45), followed by two-way agglomerative clustering of the 108 resulting probe sets and 76 samples. This analysis identified three discrete groups of sample sets that differ markedly in their expression of the survival-related genes (FIG. 1A).

In order to develop a set of markers for each of the three tumor subclasses, we identified probe sets most strongly overexpressed by tumors within each subgroup as compared to tumors in the remaining subclasses (see Table A: Glioma Determinative Markers). Using the most robust markers for each of the three tumor subsets, we derived a set of 35 genes, referred to as signature genes, that can be used in either hierarchical clustering (FIG. 1B) or k-means clustering to assign tumors to subclass. The tumor subclasses defined by k-means clustering are designated proneural (PN), proliferative (Proliif and mesenchymal (Mes) to recognize the dominant feature of the marker gene list that characterizes each subclass. For each tumor subclass, a centroid was calculated from the average expression values of the 35 signature genes (FIG. 1B). A centroid can be viewed as the prototypical expression pattern for a tumor subtype and additional samples can be classified based on the similarity of their expression of the signature genes to these centroids. Kaplan-Meier plots for cases in the MDA dataset showed that median survival of the PN subclass (174.5 wks) was markedly longer than either Prolif (60.5) or Mes subtypes (65.0 wks; FIG. 1C).

To demonstrate the prognostic value of the novel tumor classification scheme, we examined two additional independent data sets. One dataset was generated from a set of AA and GBM samples obtained from cases treated at University of California San Francisco (UCSF) while the second validation dataset was obtained from a previously published study on cases of grade III & IV tumors of astrocytic, oligodendroglial., and mixed morphologies seen at UCLA (Freije et al., 2004). Tumors in both validation sets were classifed into PN, Prolif, and Mes subtypes based on similarity of their expression of the signature genes to the centroids defined in the MDA data set. Kaplan-Meier plots for survival of tumor subtypes in both validation sets showed a very similar pattern to that observed in the initial dataset (FIG. 1 D&E). Considering only GBM cases, the prognostic value of the distinction between PN vs. other classes was seen to be statistically significant either within the MDA sample set or across all 3 datasets combined (p<0.05, t test for both comparisons of PN vs. other classes).

We next compared expression of selected PN and Mes markers by both microarray and quantitative real-time PCR. We selected DLL3 (delta-like ligand 3) and CHI3L1/YKL40 as PN and Mes markers, respectively, from the signature gene list and BCAN and CD44 from the more extensive list of markers (Table A) for these same two subclasses, respectively. As seen in FIG. 1F & G, most glioma samples displayed expression values above the population mean for either the pair of PN markers or the pair Mes markers, but rarely for combinations of PN and Mes markers.

In situ hybridization on a set of independent tumor cases confirmed a mutually-exclusive pattern of expression for BCAN and CHI3L1/YKL40 mRNAs (FIG. 1H). Most specimens displayed strong signal for either BCAN or CHI3L1/YKL40, but not both markers. Some specimens did, however, display focal expression of each of the two markers in non-overlapping cellular elements. Most typically, these were specimens with small foci of CHI3L1/YKL40 expression in specimens containing broad BCAN positive regions. In such cases, CHI3L1/YKL40 expression was frequently associated with blood vessels.

PN, Prolif, and Mes Signatures Define Tumor Subpopulations That Differ in Tumor Grade and Patient Age

Expanding our analysis to include a total of 256 high-grade gliomas (supp. table 1), we observed that most tumors displayed a strong similarity to either the PN, the Prolif or the Mes centroid and a neutral or negative correlation with the other two centroids (FIG. 2A). This finding is reflected by the tendency of samples to cluster at the apices of a triangle in the three-dimensional plot displayed in FIG. 2A. While some samples showed intermediate similarity to two centroids, very few samples showed weak or neutral similarities to all three centroids. Thus, most high-grade gliomas tend to reside in one of three discrete phenotypic states, but may more rarely exist in a condition that is intermediate between two conditions.

The newly-defined tumor subclasses displayed a strong association with histological tumor grade. Nearly all grade III tumor specimens examined (89%) were classified as PN regardless of whether they exhibited oligodendroglial or astrocytic morphology. In contrast, a significant proportion of grade IV lesions (GBM) was classified into each of the three molecular categories. Of 184 GBM samples examined, 32% were PN, 20% Prolif, and 48% Mes.

Qualitative examination of H&E stained sections from the GBM cases included I the MDA sample set revealed no histological feature that uniformly distinguished the molecular sublasses of tumor. The only morphological feature whose occurrence is statistically associated with molecular subclass was tumor necrosis, which was less frequent in PN tumors. Among 9 GBM cases identified as PN, 4 lacked any necrotic regions. In contrast, only 2 of 22 Mes GBMs (p<0.05, PN vs. Mes, Fischer's exact test) and 0 of 24 Prolif GBMs (p=0.005, PN vs. Prolif) failed to display necrosis.

Consistent with well-established correlations of both tumor grade and survival time to patient age, we found that assignment of tumors to molecular subtypes stratifies patients on the basis of age. Among the 185 newly-diagnosed cases in our survival analyses, patients with tumors in the PN subclass were significantly younger than those with tumors in either Prolif or Mes subclasses (p<0.005 t-tests for both comparisons). Mean age +/− SEM for patients with tumors of PN, Prolif and Mes subclass was 40.5 +/− 1.4 yrs, 49.0 +/− 2.5 yrs, and 50.7 +/− 1.3 yrs, respectively. For GBMs, Mes cases were significantly older than PN (p <0.05, t test) while Prolif cases did not differ in age from either of the other 2 classes.

PN, Prolif, and Mes Signatures Characterize Distinct Sets of Normal Tissues

To gain insights into the biological significance of the molecular signatures represented by PN, Prolif and Mes centroids, we examined expression of the 35 signature genes in several human tissue and cell types. This analysis revealed that distinct sets of tissues resemble each of the centroids that define glioma subclasses (FIG. 2B). Both fetal and adult brain had a positive correlation with the PN (proneural) centroid. Two neural stem cell lines derived from fetal human brain also displayed a PN signature under normal culture conditions (FIG. 2B). Tissues displaying the Mes (mesenchymal) signature included bone, synovium, smooth muscle, endothelial cells, and dendritic cells. In addition, a sample of cultured human fetal astroctyes displayed a clear positive association with the Mes centroid. Both hematopoietic stem cells isolated from peripheral blood and the highly proliferative cell line Jurkat had a strong association with the Prolif centroid. Interestingly, the two neural stem cell lines shifted signature subclass from the PN to the Mes class in response to treatment and withdrawal of the neurotrophic factor BDNF (FIG. 2B).

Upon Recurrence, Tumors Tend to Shift Towards the Mes Phenotype

To determine if the molecular signatures that define tumor subclasses are a fixed feature of each tumor case or may change as a function of treatment or disease progression, we compared expression signatures of 26 pairs of matched specimens that represent primary and recurrent astrocytomas from the same pateients. The mean change in signature in all 26 primary vs. recurrent sample pairs corresponded to a loss in similarity to the PN centroid of 0.18 +/− 0.06 (mean shift in Pearson r +/− SEM), a gain in similarity to the Mes centroid of 0.20 +/− 0.09 and very little change in similarity to the Prolif centroid (gain of 0.02 +/− 0.08). 18 of the 26 cases arose and recurred in the same molecular subclass while eight tumors changed class upon recurrence (FIG. 2C). Of these eight cases of phenotypic class shift, all but one represent shifts into the Mes subclass; three cases shifted from PN to Mes subtype and four switched from Prolif to Mes phenotype. The final instance of class shift is a case that moved from moderate Mes similarity to moderate Prolif similarity.

Using a pair-wise analysis, significance analysis of microarrays (SAM) identified genes that were significantly upregulated in the recurrent tumors that switched into the Mes subclass (FIG. 2D). Upregulated genes included CHI3L1/YKL-40, CD44, and STAT3, genes previously implicated in GBM biology as well as vimentin (VIM), a classic marker of mesenchymal tissues (Eibl et al., J. Neuro-Oncol. 26: 165-170 (1995); Nigro et al., supra.; Nutt et al., Cancer Res. 63: 1602-1607 (2005); Rahaman et al., Oncogene 21: 8404-8413 (2002); Tanwar et al., Cancer Res. 62: 4364-4368 (2002). CHI3L1/YKL-40 is reported to predict radioresistance in human tumors (Pelloski et al., Clin. Cancer Res. 11: 3326-3334 (2005) and to promote clonogenic survival following radiation in vitro (Nigro et al., supra.), findings consistent with a relative increase in expression upon tumor recurrence after treatment. No genes were found to be significantly downregulated in cases that shift into Mes class.

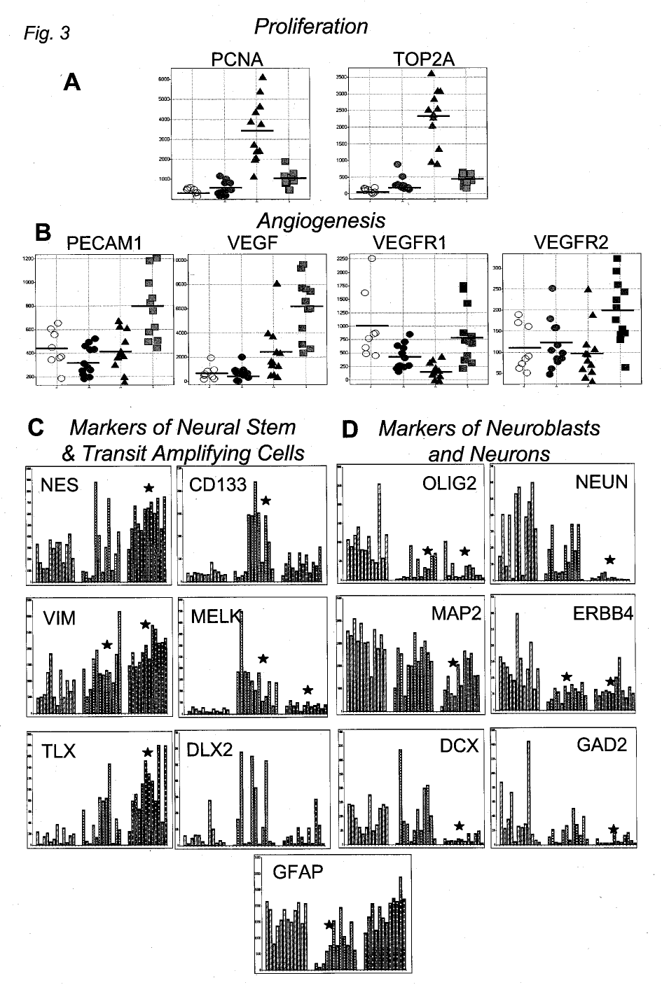

Immunohistochemistry on tissue from cases that shifted away from PN upon recurrence suggested frequent loss of prominent nuclear OLIG2 expression and upregulation of CHI3L1/YKL-40. In the example shown in FIG. 3, a PN tumor showing prominent nuclear expression of OLIG2, a PN marker previously reported to be preferentially associated with low grade lesions (Ligon et al., J. Neuropathol. Exp. Neurol. 63: 499-509 (2004), exhibited relative loss of OLIG2 expression at recurrence. Normal brain exhibited only low level staining in oligodendroglia, indicating that upregulation of this marker by PN tumors in array data is not a manifestation of normal brain contamination during tissue processing. Conversely, while CHI3L1/YKL-40, a Mes marker, was expressed only in rare tumor cells in the primary sample, abundant expression is seen in the recurrence. Such changes in relative expression were seen in all tumors examined with a Mes shift at recurrence.

Poor Prognosis Tumor Subtypes are Distinguished by Markers of Proliferation or Angiogenesis

Having defined prognostic subclasses of tumor, we sought to identify aspects of biology that might contribute to differences in disease aggressiveness. To examine the phenotypes of tumor subclasses, we selected sets of astrocytoma cases from our survival analyses that showed the strongest similarity to the centroids used for classification (n=12 per subtype). We included in our comparison eight normal brain tissue samples. In examining markers of proliferation, we found that both proliferating cell nuclear antigen (PCNA) and topoisomerase II alpha (TOP2A), were significantly overexpressed in the Prolif tumors as compared to the PN or Mes tumors (FIG. 3A). One possible mechanism by which Mes tumors manifest an aggressive phenotype in the absence of a high rate of cell division is through angiogenesis. As seen in FIG. 3B, Mes tumors displayed overexpression of vascular endothelial growth factor (VEGF), flt1 or VEGF receptor 1 (VEGFR1), kdr or VEGF receptor 2 (VEGFR2), and the endothelial marker PECAM1.

Poor Prognosis Tumor Subtypes Express Markers of Neural Stem and/or Transit Amplifying Cells While Better Prognosis Tumor Express Markers of Neuroblasts or Neurons

Some of the PN markers in the signature gene set, such as NCAM, GABBR1 and SNAP91 are associated with neurons. In light of recent findings that tumorigenic cells of GBM express the neural stem cell marker CD133 (Singh et al., Nature 432: 396-401 (2004), we sought to compare the expression of markers for neural stem cells vs. markers of committed neuronal lineage (FIG. 3C-E). For our analysis, we selected markers associated with adult forebrain neurogenesis (Abrous et al., Physiol. Rev. 85: 523-569 (2005); Anton et al., Nature Neurosci. 7: 1319-1328 (2004); Nakano et al., J. Cell Biol. 170: 413 (2005); Shi et al., Nature 427: 78-83 (2004). For five out of six markers of neural stem cells or multipotent transit-amplifying cells, we found that one or both of the poor prognosis tumor subclasses showed elevated expression compared to PN tumors (FIG. 3C). Such differences were seen with vimentin (VIM), nestin (NES), TLX, CD133, and MELK. While DLX2, a marker of transit-amplifying cells, did not show statistically significant differences between tumor groups, some Prolif tumors showed strong expression of this gene. In contrast stem cell markers, markers of neuroblasts or developing neurons were overexpressed in PN tumors as compared to Prolif and/or Mes tumors (FIG. 3D). These markers include OLIG2, MAP2, DCX, ENC 1 (NeuN), ERBB4, and GAD2. Expression of neuronal markers in PN tumors was not accounted for by contamination of tumor specimens with normal brain, as expression levels of OLIG2, DCX, NeuN, and GAD2 were elevated in tumors as compared to normal brain specimens (data not shown, p<0.005 for all t test comparisons of tumor vs normal., uncorrected for multiple comparisons). GFAP, a marker of both neural stem cells and astrocytes was more strongly expressed in tumors of both Mes and PN subclasses as compared to Prolif tumors (FIG. 3E).

Losses on chr 10 and Gains on chr 7 are Associated with Prolif and Mes Tumor Subtypes

Of the cases examined by expression profiling, DNA from 96 specimens of AA and GBM was available for analysis by array comparative genomic hybridization (CGH). Tumors were scored for copy number changes on chrs 1, 7, 10 and 19. Tumors were scored for relative copy number changes on chrs 1, 7, 10 and 19. The majority of samples demonstrated gains on chr 7 and losses on chr 10 (FIG. 4A). In examining losses on chr 10, we found striking differences in the frequency of these genomic alterations between tumor subclasses (FIG. 4A). While the majority of Prolif and Mes tumors had losses on chr 10 that span 10q23.3 (78% and 84% respectively), a minority (20%) of the PN tumor subclass showed losses on chr 10. For Mes tumors, most cases lost essentially all loci on chr 10, and only two cases had losses confined to 10q. In contrast, Prolif tumors had more heterogeneous losses on Chr 10. The association between prognostic signatures and status of Chr 10 is highly significant (p<0.0001, Fisher's exact test). Similar but less robust associations between tumor signature and relative copy number changes on chr 7 or 19q (FIG. 4A, p <0.01 for both chr7 and 19q by Fisher's exact test), but not on chr1p or 1q (p >0.05).

Given the association between relative genomic copy number changes and tumor subtypes, we sought to determine whether the genes that define each tumor subtype were biased for chromosomal location. Chi square analysis on the extended lists of tumor subclass markers (Table A) revealed that for each of the three lists, the observed frequencies of chromosome locations differed significantly from that expected by the frequencies of locations for all probe sets on the expression arrays (p<1×10⁻¹⁴ for PN, p <0.001 for Prolif, p<0.0005 for Mes). PN and Mes marker lists overrepresent markers on chr10 and 19, respectivelyly (P<0.05 for both comparisons after Bonferroni correction for 72 comparisons). These findings corroborate the differences between tumor subtypes in relative DNA copy number changes on chr10 and 19q.

Notch and Akt Pathways are Differentially Activated in Tumor Subclasses with Good Vs. Poor Prognosis

Consistent with the association of chr10 loss and tumor subclass, direct examination of array CGH data for the BAC clones that encompass the PTEN locus confirmed that a high percentage of Prolif and Mes cases displayed losses of the PTEN locus. A negative association was seen between losses of the PTEN locus and similarity to the PN centroid (FIG. 5A). Most cases of gains or amplifications of the locus were tumors of either Prolif or Mes subclasses, and a negative correlation was seen between EGFR copy number gains and similarity to the PN centroid (FIG. 5B). No obvious amplifications or deletions were seen at loci corresponding to akt1, akt2, or akt3, nor of catalytic subunits of P13K (not shown). A small number of samples demonstrated gains in copy number for PIK3R3, a regulatory subunit of PI3K, and CGH ratios for this locus were positively correlated with similarity to the Prolif signature (FIG. 5C).

Since our results suggested strong relationships between akt pathway activation and tumor subtypes, we compared PTEN mRNA expression and phospho-akt (p-akt) immunohistochemistry in tumor subtypes. We found that Prolif and Mes tumors expressed approximately 2 fold lower PTEN mRNA and stronger p-akt (ser473) as compared to PN tumors (FIG., 4E; FIG. 4J). The results were highly significant (p<5×10⁻⁵ for PTEN mRNA; p<5×10⁻⁸ for p-akt immunohistochemistry for t tests of PN vs. other). The PTEN results were validated in a second independent sample set (data not shown).

In examining the complete list of PN tumor markers in the MDA sample set, we found that probe sets corresponding to the Notch pathway elements DLL3, DLL 1, HEY2 and ASCL1 met our criteria for markers of PN tumors (FIG. 5 E-H). These genes were confirmed to be significantly overexpressed in PN tumors of both validation datasets. Each of these four Notch pathway elements has been implicated in forebrain neurogenesis. (Campos et al., J. Neurosci. Res. 64: 590-598 (2001); Casarosa et al., Development 126: 525-534 (1999); Sakamoto et al., J. Biol. Chem. 278: 44808-44815 (2003) and ASCL1 has recently been linked to specification of both neurons and oligodendroglia in the adult forebrain. (Parras et al., EMBO Journal, 23(22): 4495-505 (2004). Elements of the Notch pathway for which microarray data did not show upregulation in PN tumors include Notch 1-4, Jagged 1 & 2, Hes 1, 2, 4, 6, 7 (not shown). HEY1 showed a small, but significant upregulation in MDA PN tumors (1.4FC, p=5×10-5).

To examine direct evidence for Notch signaling activation we utilized a blinded assessment of nuclear Notch-immunoreactivity in all MDA cases with available paraffin-embedded sections. Positive cases showed a “blush” in the nucleus, as compared to negative cases, which showed a complete absence of nuclear staining. FIG. 4K displays frequencies of each tumor subtype rated 0, 1, or 2 for Notch nuclear staining. In spite of the relatively weak signal exhibited by positive cases, ratings of PN samples were demonstrated to be significantly higher than those of either Prolif (p<0.005, t test) or Mes (p<0.001, t test) subclasses.

A Two-Gene Model of DLL3 and PTEN Expression Predicts Survival of High-Grade Astrocytoma

Since our data suggested a role for Notch and akt pathways in tumor aggressiveness, we sought evidence for a direct association between expression of pathway markers and survival. For this analysis, we evaluated PTEN mRNA by quantitative PCR and DLL3 mRNA by microarray data from all samples in the MDA survival set where sufficient mRNA was available (n=65). A Cox model of proportional hazards revealed that levels of PTEN and DLL3 mRNA, and their statistical interaction were all associated with survival in high-grade astrocytoma, and combined to form a highly significant predictive model (Likelihood Ratio Test of 21.6 compared to a chi-square reference distribution with 3 degrees of freedom, p<0.0001). The predicted survival functions depicted in FIG. 5A demonstrate that samples with low values of PTEN mRNA were associated with a poor survival function regardless of the level of DLL3 expression. For samples with high PTEN expression, estimated survival was seen to vary as a function of DLL3 such that samples with high levels of both PTEN and DLL3 mRNAs showed the best outcomes. We next fitted the same model with a smaller (n=34) independent set of grade III & IV astrocytomas. Predicted survival curves are strikingly similar to those obtained from the MDA sample set (FIG., 5B), indicating the robustness of this two-gene model (FIG. 6B).

Expression Signatures of GBM Cell Lines Predict EGF/FGF Independent Neurosphere Growth and Sensitivity to Akt Pathway Inhibition

To examine the potential therapeutic significance of the molecular subtypes of tumor, we examined responses in vitro of glioma cell lines as a function of their expression of the signature genes. We profiled 16 GBM cell lines for mRNA expression, investigated their ability to generate neurospheres in the presence or absence of EGF+FGF, and evaluated their response to agents that affect Notch and Akt signaling. All 16 lines were negatively correlated with respect to the PN centroid, but showed a wide range of similarities to the Mes centroid. While 15 of the 16 cell lines generated neurospheres that could be propagated in EGF+FGF, their ability to generate neurospheres that grow in the absence of EGF+FGF varied (FIG. 6A), and appeared to be related to the expression signature of the parental cell line (FIG. 6B). Most strikingly, we found that two cells lines negatively correlated to the Mes centroid (G112 & G122) generated neurospheres that grew rapidly in the absence of EGF+FGF (FIG. 6B), while cell lines with a strong correlation to the Mes centroid failed to generate neurospheres that could be readily propagated in the absence of EGF+FGF (FIG. 7B).

A summary of major findings, including the parallels between tumor subtypes and stages in forebrain neural development, is displayed in FIGS. 8 A & B.

### Example 2

Microarray Analysis to Detect Upregulation of GDM Polypeptides in Cancerous Glioma Tumors

Nucleic acid microarrays, often containing thousands of gene sequences, are useful for identifying differentially expressed genes in diseased tissues as compared to their normal counterparts. Using nucleic acid microarrays, test and control mRNA samples from test and control tissue samples are reverse transcribed and labeled to generate cDNA probes. The cDNA probes are then hybridized to an array of nucleic acids immobilized on a solid support. The array is configured such that the sequence and position of each member of the array is known. For example, a selection of genes known to be expressed in certain disease states may be arrayed on a solid support. Hybridization of a labeled probe with a particular array member indicates that the sample from which the probe was derived expresses that gene. If the hybridization signal of a probe from a test (disease tissue) sample is greater than hybridization signal of a probe from a control (normal tissue) sample, the gene or genes overexpressed in the disease tissue are identified. The implication of this result is that an overexpressed protein in a diseased tissue is useful not only as a diagnostic marker for the presence of the disease condition, but also as a therapeutic target for treatment of the disease condition.

The methodology of hybridization of nucleic acids and microarray technology is well known in the art. In the present example, the specific preparation of nucleic acids for hybridization and probes, slides, and hybridization conditions are all detailed in PCT Patent Application Ser. No. PCT/US01/10482, filed on Mar. 30, 2001 and which is herein incorporated by reference.

### Example 3

Quantitative Analysis of GDM mRNA Expression

In this assay, a 5′ nuclease assay (for example, TaqMan®) and real-time quantitative PCR (for example, ABI Prizm 7700 Sequence Detection System® (Perkin Elmer, Applied Biosystems Division, Foster City, Calif.)), is used to find genes that are significantly overexpressed in a cancerous glioma tumor or tumors as compared to other cancerous tumors or normal non-cancerous tissue. The 5′ nuclease assay reaction is a fluorescent PCR-based technique which makes use of the 5′ exonuclease activity of Taq DNA polymerase enzyme to monitor gene expression in real time. Two oligonucleotide primers (whose sequences are based upon the gene or EST sequence of interest) are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the PCR amplification reaction, the Taq DNA polymerase enzyme cleaves the probe in a template-dependent manner. The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative and quantitative interpretation of the data. This assay is well known and routinely used in the art to quantitatively identify gene expression differences between two different human tissue samples, see, e.g., Higuchi et al., Biotechnology 10:413-417 (1992); Livak et al., PCR Methods Appl., 4:357-362 (1995); Heid et al., Genome Res. 6:986-994 (1996); Pennica et al., Proc. Natl. Acad. Sci. USA 95(25):14717-14722 (1998); Pitti et al., Nature 396(6712):699-703 (1998) and Bieche et al., Int. J. Cancer 78:661-666 (1998).

The 5′ nuclease procedure is run on a real-time quantitative PCR device such as the ABI Prism 7700TM Sequence Detection. The system consists of a thermocycler, laser, charge-coupled device (CCD) camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

The starting material for the screen is mRNA isolated from a variety of different cancerous tissues. The mRNA is quantitated precisely, e.g., fluorometrically. As a negative control, RNA is isolated from various normal tissues of the same tissue type as the cancerous tissues being tested. Frequently, tumor sample(s) are directly compared to “matched” normal sample(s) of the same tissue type, meaning that the tumor and normal sample(s) are obtained from the same individual.

5′ nuclease assay data are initially expressed as Ct, or the threshold cycle. This is defined as the cycle at which the reporter signal accumulates above the background level of fluorescence. The ΔCt values are used as quantitative measurement of the relative number of starting copies of a particular target sequence in a nucleic acid sample when comparing cancer mRNA results to normal human mRNA results. As one Ct unit corresponds to 1 PCR cycle or approximately a 2-fold relative increase relative to normal., two units corresponds to a 4-fold relative increase, 3 units corresponds to an 8-fold relative increase and so on, one can quantitatively and quantitatively measure the relative fold increase in mRNA expression between two or more different tissues. In this regard, it is well accepted in the art that this assay is sufficiently technically sensitive to reproducibly detect an at least 2-fold increase in mRNA expression in a human tumor sample relative to a normal control.

### Example 4

In situ Hybridization

In situ hybridization is a powerful and versatile technique for the detection and localization of nucleic acid sequences within cell or tissue preparations. It may be useful, for example, to identify sites of gene expression, analyze the tissue distribution of transcription, identify and localize viral infection, follow changes in specific mRNA synthesis and aid in chromosome mapping.

In situ hybridization is performed following an optimized version of the protocol by Lu and Gillett, Cell Vision 1:169-176 (1994), using PCR-generated ³³P-labeled riboprobes. Briefly, formalin-fixed, paraffin-embedded human tissues are sectioned, deparaffinized, deproteinated in proteinase K (20 g/ml) for 15 minutes at 37° C., and further processed for in situ hybridization as described by Lu and Gillett, supra. A [³³-p] UTP-labeled antisense riboprobe are generated from a PCR product and hybridized at 55° C. overnight. The slides are dipped in Kodak NTB2 nuclear track emulsion and exposed for 4 weeks.

³³P-Riboprobe Synthesis

6.0 μl (125 mCi) of ³³P-UTP (Amersham BF 1002, SA<2000 Ci/mmol) were speed vac dried. To each tube containing dried ³³P-UTP, the following ingredients were added:

2.0 μl 5× transcription buffer

1.0 μl DTT (100 mM)

2.0 μl NTP mix (2.5 mM:10 μ; each of 10 mM GTP, CTP & ATP+10 μl H₂O)

1.0 μl UTP (50 μM)

1.0 μl Rnasin

1.0 μl DNA template (1 μg)

1.0 μl H₂O

1.0 μl RNA polymerase (for PCR products T3=AS, T7=S, usually)

The tubes are incubated at 37° C. for one hour. 1.0 μl RQ1 DNase is added, followed by incubation at 37° C. for 15 minutes. 90 μl TE (10 mM Tris pH 7.6/1 mM EDTA pH 8.0) are added, and the mixture was pipetted onto DE81 paper. The remaining solution is loaded in a Microcon-50 ultrafiltration unit, and spun using program 10 (6 minutes). The filtration unit is inverted over a second tube and spun using program 2 (3 minutes). After the final recovery spin, 100 μl TE is added. 1 μl of the final product is pipetted on DE81 paper and counted in 6 ml of Biofluor II.

The probe is run on a TBE/urea gel. 1-3 μl of the probe or 5 μl of RNA Mrk III is added to 3 μl of loading buffer. After heating on a 95° C. heat block for three minutes, the probe is immediately placed on ice. The wells of gel are flushed, the sample loaded, and run at 180-250 volts for 45 minutes. The gel is wrapped in saran wrap and exposed to XAR film with an intensifying screen in −70° C. freezer one hour to overnight.

³³P-Hybridization

A. Pretreatment of Frozen Sections

The slides are removed from the freezer, placed on aluminium trays and thawed at room temperature for 5 minutes. The trays are placed in 55° C. incubator for five minutes to reduce condensation. The slides are fixed for 10 minutes in 4% paraformaldehyde on ice in the fume hood, and washed in 0.5×SSC for 5 minutes, at room temperature (25 ml 20×SSC+975 ml SQ H₂O). After deproteination in 0.5 μg/ml proteinase K for 10 minutes at 37° C. (12.5 μl of 10 mg/ml stock in 250 ml prewarmed RNase-free RNAse buffer), the sections are washed in 0.5×SSC for 10 minutes at room temperature. The sections are dehydrated in 70%, 95%, 100% ethanol, 2 minutes each.

B. Pretreatment of Paraffin-Embedded Sections

The slides are deparaffinized, placed in SQ H₂O, and rinsed twice in 2×SSC at room temperature, for 5 minutes each time. The sections are deproteinated in 20 μg/ml proteinase K (500 μl of 10 mg/ml in 250 ml RNase-free RNase buffer; 37° C., 15 minutes) —human embryo, or 8 × proteinase K (100 μl in 250 ml Rnase buffer, 37° C., 30 minutes) —formalin tissues. Subsequent rinsing in 0.5×SSC and dehydration are performed as described above.

C. Prehybridization

The slides are laid out in a plastic box lined with Box buffer (4×SSC, 50% formamide)—saturated filter paper.

D. Hybridization

1.0×10⁶ cpm probe and 1.0 μl tRNA (50 mg/ml stock) per slide are heated at 95° C. for 3 minutes. The slides are cooled on ice, and 48 μl hybridization buffer are added per slide. After vortexing, 50 μl ³³P mix are added to 50 μl prehybridization on slide. The slides are incubated overnight at 55° C.

E. Washes

Washing is done 2×10 minutes with 2×SSC, EDTA at room temperature (400 ml 20×SSC+16 ml 0.25 M EDTA, V_{f}=4L), followed by RNaseA treatment at 37° C. for 30 minutes (500 μl of 10 mg/ml in 250 ml Rnase buffer=20 μg/ml). The slides are washed 2×10 minutes with 2×SSC, EDTA at room temperature. The stringency wash conditions can be as follows: 2 hours at 55° C., 0.1×SSC, EDTA (20 ml 20×SSC+16 ml EDTA, V_{f}=4L).

F. Oligonucleotides

In situ analysis is performed on a variety of DNA sequences disclosed herein. The oligonucleotides employed for these analyses is obtained so as to be complementary to the nucleic acids (or the complements thereof) as shown in the accompanying figures.

### Example 5

Preparation of Antibodies that Bind GDM

Techniques for producing monoclonal antibodies are known in the art and are described, for instance, in Goding, supra. Immunogens that may be employed include purified GDM polypeptides, fusion proteins containing GDM polypeptides, and cells expressing recombinant GDM polypeptides on the cell surface. Selection of the immunogen can be made by the skilled artisan without undue experimentation.

Mice, such as Balb/c, are immunized with the above immunogen emulsified in complete Freund's adjuvant and injected subcutaneously or intraperitoneally in an amount from 1-100 micrograms. Alternatively, the immunogen is emulsified in MPL-TDM adjuvant (Ribi Immunochemical Research, Hamilton, Mont.) and injected into the animal's hind foot pads. The immunized mice are then boosted 10 to 12 days later with additional immunogen emulsified in the selected adjuvant. Thereafter, for several weeks, the mice may also be boosted with additional immunization injections. Serum samples may be periodically obtained from the mice by retro-orbital bleeding for testing in ELISA assays to detect anti-GDM antibodies.

After a suitable antibody titer has been detected, the animals “positive” for antibodies can be injected with a final intravenous injection of GDM polypeptide. Three to four days later, the mice are sacrificed and the spleen cells are harvested. The spleen cells are then fused (using 35% polyethylene glycol) to a selected murine myeloma cell line such as P3X63AgU.1, available from ATCC, No. CRL 1597. The fusions generate hybridoma cells which can then be plated in 96 well tissue culture plates containing HAT (hypoxanthine, aminopterin, and thymidine) medium to inhibit proliferation of non-fused cells, myeloma hybrids, and spleen cell hybrids.

The hybridoma cells are screened in an ELISA for reactivity against GDM. Determination of “positive” hybridoma cells secreting the desired monoclonal antibodies against GDM is within the skill in the art.

The positive hybridoma cells can be injected intraperitoneally into syngeneic Balb/c mice to produce ascites containing the anti-GDM monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 6

Preparation of Toxin-Conjugated Antibodies that Bind GDM

The use of antibody-drug conjugates (ADC), i.e. immunoconjugates, for the local delivery of cytotoxic or cytostatic agents, i.e. drugs to kill or inhibit tumor cells in the treatment of cancer (Payne (2003) Cancer Cell 3:207-212; Syrigos and Epenetos (1999) Anticancer Research 19:605-614; Niculescu-Duvaz and Springer (1997) Adv. Drug Del. Rev. 26:151-172; US 4,975,278) allows targeted delivery of the drug moiety to tumors, and intracellular accumulation therein, where systemic administration of these unconjugated drug agents may result in unacceptable levels of toxicity to normal cells as well as the tumor cells sought to be eliminated (Baldwin et al., (1986) Lancet (Mar. 15, 1986) pp. 603-05; Thorpe, (1985) “Antibody Carriers Of Cytotoxic Agents In Cancer Therapy: A Review,” in Monoclonal Antibodies ′84: Biological And Clinical Applications, Pinchera et al. (eds.), pp. 475-506). Maximal efficacy with minimal toxicity is sought thereby. Efforts to design and refine ADC have focused on the selectivity of monoclonal antibodies (mAbs) as well as drug-linking and drug-releasing properties. Both polyclonal antibodies and monoclonal antibodies have been reported as useful in these strategies (Rowland et al., (1986) Cancer Immunol. Immunother., 21:183-87). Drugs used in these methods include daunomycin, doxorubicin, methotrexate, and vindesine (Rowland et al., (1986) supra). Toxins used in antibody-toxin conjugates include bacterial toxins such as diphtheria toxin, plant toxins such as ricin, small molecule toxins such as geldanamycin (Mandler et al. (2000) J. of the Nat. Cancer Inst. 92(19):1573-1581; Mandler et al. (2000) Bioorganic & Med. Chem. Letters 10:1025-1028; Mandler et al. (2002) Bioconjugate Chem. 13:786-791), maytansinoids (EP 1391213; Liu et al., (1996) Proc. Natl. Acad. Sci. USA 93:8618-8623), and calicheamicin (Lode et al. (1998) Cancer Res. 58:2928; Hinman et al. (1993) Cancer Res. 53:3336-3342).

Techniques for producing antibody-drug conjugates by linking toxins to purified antibodies are well known and routinely employed in the art. For example, conjugation of a purified monoclonal antibody to the toxin DM1 may be accomplished as follows. Purified antibody is derivatized with N-succinimidyl-4-(2-pyridylthio)-pentanoate to introduce dithiopyridyl groups. Antibody (376.0 mg, 8 mg/mL) in 44.7 ml of 50 mM potassium phosphate buffer (pH 6.5) containing NaCl (50 mM) and EDTA (1 mM) is treated with SPP (5.3 molar equivalents in 2.3 ml ethanol). After incubation for 90 minutes under argon at ambient temperature, the reaction mixture is gel filtered through a Sephadex G25 column equilibrated with 35 mM sodium citrate, 154 mM NaCl and 2 mM EDTA. Antibody containing fractions are then pooled and assayed. Antibody-SPP-Py (337.0 mg with releasable 2-thiopyridine groups) is diluted with the above 35 mM sodium citrate buffer, pH 6.5, to a final concentration of 2.5 mg/ml. DM1 (1.7 equivalents, 16.1 mols) in 3.0 mM dimethylacetamide (DMA, 3% v/v in the final reaction mixture) is then added to the antibody solution. The reaction is allowed to proceed at ambient temperature under argon for 20 hours. The reaction is loaded on a Sephacryl S300 gel filtration column (5.0 cm×90.0 cm, 1.77 L) equilibrated with 35 mM sodium citrate, 154 mM NaCl, pH 6.5. The flow rate is 5.0 ml/min and 65 fractions (20.0 ml each) are collected. Fractions are pooled and assayed, wherein the number of DM1 drug molecules linked per antibody molecule (p′) is determined by measuring the absorbance at 252 nm and 280 nm.

For illustrative purposes, conjugation of a purified monoclonal antibody to the toxin DM1 may also be accomplished as follows. Purified antibody is derivatized with (Succinimidyl 4-(N-maleimidomethyl) cyclohexane-1-carboxylate (SMCC, Pierce Biotechnology, Inc) to introduce the SMCC linker. The antibody is treated at 20 mg/ml in 50 mM potassium phosphate/50 mM sodium chloride/2 mM EDTA, pH 6.5 with 7.5 molar equivalents of SMCC (20 mM in DMSO, 6.7 mg/ml). After stirring for 2 hours under argon at ambient temperature, the reaction mixture is filtered through a Sephadex G25 column equilibrated with 50 mM potassium phosphate/50 mM sodium chloride/2 mM EDTA, pH 6.5. Antibody containing fractions are pooled and assayed. Antibody-SMCC is then diluted with 50 mM potassium phosphate/50 mM sodium chloride/2 mM EDTA, pH 6.5, to a final concentration of 10 mg/ml, and reacted with a 10 mM solution of DM1 (1.7 equivalents assuming 5 SMCC/antibody, 7.37 mg/ml) in dimethylacetamide. The reaction is stirred at ambient temperature under argon 16.5 hours. The conjugation reaction mixture is then filtered through a Sephadex G25 gel filtration column (1.5×4.9 cm) with 1× PBS at pH 6.5. The DM1/antibody ratio (p) is then measured by the absorbance at 252 nm and at 280 nm.

Cytotoxic drugs have typically been conjugated to antibodies through the often numerous lysine residues of the antibody. Conjugation through thiol groups present, or engineered into, the antibody of interest has also been accomplished. For example, cysteine residues have been introduced into proteins by genetic engineering techniques to form covalent attachment sites for ligands (Better et al. (1994) J. Biol. Chem. 13:9644-9650; Bernhard et al. (1994) Bioconjugate Chem. 5:126-132; Greenwood et al. (1994) Therapeutic Immunology 1:247-255; Tu et al. (1999) Proc. Natl. Acad. Sci USA 96:4862-4867; Kanno et al. (2000) J. of Biotechnolo2y, 76:207-214; Chmura et al. (2001) Proc. Nat. Acad. Sci. USA 98(15):8480-8484; U.S. Pat. No. 6,248,564). Once a free cysteine residue exists in the antibody of interest, toxins can be linked to that site. As an example, the drug linker reagents, maleimidocaproyl-monomethyl auristatin E (MMAE), i.e. MC-MMAE, maleimidocaproyl-monomethyl auristatin F (MMAF), i.e. MC-MMAF, MC-val-cit-PAB-MMAE or MC-val-cit-PAB-MMAF, dissolved in DMSO, is diluted in acetonitrile and water at known concentration, and added to chilled cysteine-derivatized antibody in phosphate buffered saline (PBS). After about one hour, an excess of maleimide is added to quench the reaction and cap any unreacted antibody thiol groups. The reaction mixture is concentrated by centrifugal ultrafiltration and the toxin conjugated antibody is purified and desalted by elution through G25 resin in PBS, filtered through 0.2m filters under sterile conditions, and frozen for storage.

Moreover, a free cysteine on an antibody of choice may be modified by the bis-maleimido reagent BM(PEO)4 (Pierce Chemical), leaving an unreacted maleimido group on the surface of the antibody. This may be accomplished by dissolving BM(PEO)4 in a 50% ethanol/water mixture to a concentration of 10 mM and adding a tenfold molar excess to a solution containing the antibody in phosphate buffered saline at a concentration of approximately 1.6 mg/ml (10 micromolar) and allowing it to react for 1 hour. Excess BM(PEO)4 is removed by gel filtration in 30 mM citrate, pH 6 with 150 mM NaCl buffer. An approximate 10 fold molar excess DM1 is dissolved in dimethyl acetamide (DMA) and added to the antibody-BMPEO intermediate. Dimethyl formamide (DMF) may also be employed to dissolve the drug moiety reagent. The reaction mixture is allowed to react overnight before gel filtration or dialysis into PBS to remove unreacted drug. Gel filtration on S200 columns in PBS is used to remove high molecular weight aggregates and furnish purified antibody-BMPEO-DM1 conjugate.

### Example 7

—In Vitro Cell Killing Assays

Mammalian cells expressing the GDM polypeptide of interest may be obtained using standard expression vector and cloning techniques. Alternatively, many tumor cell lines expressing GDM polypeptides of interest are publicly available, for example, through the ATCC and can be routinely identified using standard ELISA or FACS analysis. Anti-GDM polypeptide monoclonal antibodies (and toxin conjugated derivatives thereof) may then be employed in assays to determine the ability of the antibody to kill GDM polypeptide expressing cells in vitro.

With specific regard to the present invention, a PC3-derived cell line that stably expresses GDM polypeptide on its cells surface (herein called PC3-gD-MDP) may be engineered using standard techniques and expression of the GDM polypeptide by the PC3-gD-MDP cells can be confirmed using standard FACS cell sorting, ELISA and immunohistochemistry analyses. The ability of an MMAE-conjugated anti-GDM monoclonal antibody to cause the death of the respective GDM-expressing cells may be determined using an in vitro cell killing assay employing the following protocol (Promega Corp. Technical Bulletin TB288; Mendoza et al (2002) Cancer Res. 62:5485-5488):
1. An aliquot of 50 μl of cell culture containing about 10⁴ cells (either PC3-gD-MDP cells or untransfected PC3 cells which do not express GDM) in growth medium is deposited in each well of a 96-well, opaque-walled plate. Additional control wells are set up which contain 50 μl of growth medium without cells.
2. The GDM-MMAE conjugated antibody, or an MMAE-conjugated control monoclonal antibody that does not bind to GDM, is added to each well in a volume of 50 μl and at various concentrations ranging from 0.0001 to 100 μg/ml and the plates are incubated at 37° C. and 5% CO₂ for 3-5 days.
3. The plates are equilibrated to room temperature for approximately 30 minutes.
4. A volume of the CellTiter-Glo Luminescent Cell Viability Reagent from Promega Corp. equal to the volume of cell culture medium present in each well is added and the plates are shaken for 2 minutes on an orbital shaker to induce cell lysis.
5. The plates are incubated at room temperature for 10 minutes to stabilize the luminescence signal.
6. Luminescence is recorded on a luminometer with the Tropix Winglow Program and reported as RLU=relative luminescence units.

The results obtained from the above described assay can demonstrate that the GDM-MMAE antibody is capable of inducing the death of cells that express the corresponding GDM polypeptide in an antibody-dependent fashion. That is, neither GDM-MMAE nor MMAE-conjugated control can induce significant death of untransfected PC3 cells at an antibody concentration of 1 μg/ml and below. At antibody concentrations above 1 μg/ml, the amount of untransfected PC3 cell death may increase linearly with antibody concentration in an antibody-independent manner. Therefore, it will appear that the death of untransfected PC3 cells at antibody concentrations above 1 μg/ml is a non-specific result of the increasing levels of the MMAE toxin present in the reaction mixture and is not a function of the binding specificity of the antibody employed.

With regard to the PC3-gD-MDP cells that stably express the GDM polypeptide, however, while the MMAE-conjugated control induces cell death with a pattern that is identical to that antibody's ability to kill untransfected PC3 cells, the GDM-MMAE will induce significant cell killing at antibody concentrations significantly below this level (e.g., as low as 0.001 μg/ml). In fact, at an antibody concentration of 1 μg/ml (where the non-GDM specific MMAE-conjugated control antibody exhibits no significant cell killing), virtually all of the PC3-gD-MDP cells will be killed by GDM-MMAE. As such, such data will demonstrate that GDM-specific monoclonal antibody binds to the GDM polypeptide as it is expressed on the surface of cells and is capable of inducing the death of those cells to which it binds.

### Example 9

In Vivo Tumor Cell Killing Assay

To test the efficacy of toxin-conjugated or unconjugated anti-GDM polypeptide monoclonal antibodies for the ability to induce the death of tumor cells in vivo, the following protocol may be employed.

Inoculate a group of athymic nude mice with 5×10⁶ of the GDM polypeptide-expressing tumor promoting cells subcutaneously in the flank. When the tumors reach a mean tumor volume of between 100-200 mm³, the mice are grouped equally into 5 groups and are treated as follows:
Group 1—PBS control vehicle administered once per week for 4 weeks;
Group 2—non-specific control antibody administered at 1 mg/kg, once per week for 4 weeks;
Group 3—non-specific control antibody administered at 3 mg/kg, once per week for 4 weeks;
Group 4—specific anti-GDM polypeptide antibody administered at 1 mg/kg, once per week for 4 weeks;
Group 5—specific anti-GDM polypeptide antibody administered at 3 mg/kg, once per week for 4 weeks.
   Mean tumor volume may then be determined in the mice of each treatment group at periodic intervals and the efficacy of the antibodies determined.

### Example 9

Use of GDM as a Hybridization Probe

The following method describes use of a nucleotide sequence encoding GDM polypeptide as a hybridization probe for, i.e., diagnosis of the presence of a tumor in a mammal.

DNA comprising the coding sequence of full-length or mature GDM polypeptide as disclosed herein can also be employed as a probe to screen for homologous DNAs (such as those encoding naturally-occurring variants of GDM) in human tissue cDNA libraries or human tissue genomic libraries.

Hybridization and washing of filters containing either library DNAs is performed under the following high stringency conditions. Hybridization of radiolabeled GDM-derived probe to the filters is performed in a solution of 50% formamide, 5×SSC, 0.1% SDS, 0.1% sodium pyrophosphate, 50 mM sodium phosphate, pH 6.8, 2×Denhardt's solution, and 10% dextran sulfate at 42° C. for 20 hours. Washing of the filters is performed in an aqueous solution of 0.1×SSC and 0.1% SDS at 42° C.

DNAs having a desired sequence identity with the DNA encoding full-length native sequence GDM polypeptide can then be identified using standard techniques known in the art.

### Example 10

Expression of GDM in E. coli

This example illustrates preparation of an unglycosylated form of GDM by recombinant expression in E. coli.

The DNA sequence encoding the preceding GDM polypeptide sequences is initially amplified using selected PCR primers. The primers should contain restriction enzyme sites which correspond to the restriction enzyme sites on the selected expression vector. A variety of expression vectors may be employed. An example of a suitable vector is pBR322 (derived from E. coli; see Bolivar et al., Gene, 2:95 (1977)) which contains genes for ampicillin and tetracycline resistance. The vector is digested with restriction enzyme and dephosphorylated. The PCR amplified sequences are then ligated into the vector. The vector will preferably include sequences which encode for an antibiotic resistance gene, a trp promoter, a polyhis leader (including the first six STII codons, polyhis sequence, and enterokinase cleavage site), the GDM coding region, lambda transcriptional terminator, and an argU gene.

The ligation mixture is then used to transform a selected E. coli strain using the methods described in Sambrook et al., supra. Transformants are identified by their ability to grow on LB plates and antibiotic resistant colonies are then selected. Plasmid DNA can be isolated and confirmed by restriction analysis and DNA sequencing.

Selected clones can be grown overnight in liquid culture medium such as LB broth supplemented with antibiotics. The overnight culture may subsequently be used to inoculate a larger scale culture. The cells are then grown to a desired optical density, during which the expression promoter is turned on.

After culturing the cells for several more hours, the cells can be harvested by centrifugation. The cell pellet obtained by the centrifugation can be solubilized using various agents known in the art, and the solubilized GDM polypeptide can then be purified using a metal chelating column under conditions that allow tight binding of the protein.

The preceding GDM polypeptide sequences may be expressed in E. coli in a poly-His tagged form, using the following procedure. The DNA encoding GDM is initially amplified using selected PCR primers. The primers will contain restriction enzyme sites which correspond to the restriction enzyme sites on the selected expression vector, and other useful sequences providing for efficient and reliable translation initiation, rapid purification on a metal chelation column, and proteolytic removal with enterokinase. The PCR-amplified, poly-His tagged sequences are then ligated into an expression vector, which is used to transform an E. coli host based on strain 52 (W3110 fuhA(tonA) Ion galE rpoHts(htpRts) clpP(lacIq). Transformants are first grown in LB containing 50 mg/ml carbenicillin at 30° C. with shaking until an O.D.600 of 3-5 is reached. Cultures are then diluted 50-100 fold into CRAP media (prepared by mixing 3.57 g (NH₄)₂SO₄, 0.71 g sodium citratee●2H2O, 1.07 g KCl, 5.36 g Difco yeast extract, 5.36 g Sheffield hycase SF in 500 mL water, as well as 110 mM MPOS, pH 7.3, 0.55% (w/v) glucose and 7 mM MgSO₄) and grown for approximately 20-30 hours at 30° C. with shaking. Samples are removed to verify expression by SDS-PAGE analysis, and the bulk culture is centrifuged to pellet the cells. Cell pellets are frozen until purification and refolding.

E. coli paste from 0.5 to 1 L fermentations (6-10 g pellets) is resuspended in 10 volumes (w/v) in 7 M guanidine, 20 mM Tris, pH 8 buffer. Solid sodium sulfite and sodium tetrathionate is added to make final concentrations of 0.iM and 0.02 M, respectively, and the solution is stirred overnight at 4° C. This step results in a denatured protein with all cysteine residues blocked by sulfitolization. The solution is centrifuged at 40,000 rpm in a Beckman Ultracentifuge for 30 min. The supernatant is diluted with 3-5 volumes of metal chelate column buffer (6 M guanidine, 20 mM Tris, pH 7.4) and filtered through 0.22 micron filters to clarify. The clarified extract is loaded onto a 5 ml Qiagen Ni-NTA metal chelate column equilibrated in the metal chelate column buffer. The column is washed with additional buffer containing 50 mM imidazole (Calbiochem, Utrol grade), pH 7.4. The protein is eluted with buffer containing 250 mM imidazole. Fractions containing the desired protein are pooled and stored at 40C. Protein concentration is estimated by its absorbance at 280 nm using the calculated extinction coefficient based on its amino acid sequence.

The proteins are refolded by diluting the sample slowly into freshly prepared refolding buffer consisting of: 20 mM Tris, pH 8.6, 0.3 M NaCl, 2.5 M urea, 5 mM cysteine, 20 mM glycine and 1 mM EDTA. Refolding volumes are chosen so that the final protein concentration is between 50 to 100 micrograms/ml. The refolding solution is stirred gently at 4° C. for 12-36 hours. The refolding reaction is quenched by the addition of TFA to a final concentration of 0.4% (pH of approximately 3). Before further purification of the protein, the solution is filtered through a 0.22 micron filter and acetonitrile is added to 2-10% final concentration. The refolded protein is chromatographed on a Poros R1/H reversed phase column using a mobile buffer of 0.1% TFA with elution with a gradient of acetonitrile from 10 to 80%. Aliquots of fractions with A280 absorbance are analyzed on SDS polyacrylamide gels and fractions containing homogeneous refolded protein are pooled. Generally, the properly refolded species of most proteins are eluted at the lowest concentrations of acetonitrile since those species are the most compact with their hydrophobic interiors shielded from interaction with the reversed phase resin. Aggregated species are usually eluted at higher acetonitrile concentrations. In addition to resolving misfolded forms of proteins from the desired form, the reversed phase step also removes endotoxin from the samples.

Fractions containing the desired folded protein are pooled and the acetonitrile removed using a gentle stream of nitrogen directed at the solution. Proteins are formulated into 20 mM Hepes, pH 6.8 with 0.14 M sodium chloride and 4% mannitol by dialysis or by gel filtration using G25 Superfine (Pharmacia) resins equilibrated in the formulation buffer and sterile filtered.

### Example 11

Expression of GDM Polypeptide in Mammalian Cells

This example illustrates preparation of a potentially glycosylated form of GDM polypeptide by recombinant expression in mammalian cells.

The vector, pRK5 (see EP 307,247, published Mar. 15, 1989), is employed as the expression vector. Optionally, DNA encoding the GDM polypeptides described herein is ligated into pRK5 with selected restriction enzymes to allow insertion of such DNA using ligation methods such as described in Sambrook et al., supra. The resulting vector is called GDM-DNA.

In one embodiment, the selected host cells may be 293 cells. Human 293 cells (ATCC CCL 1573) are grown to confluence in tissue culture plates in medium such as DMEM supplemented with fetal calf serum and optionally, nutrient components and/or antibiotics. About 10 μg pRK5-GDM DNA is mixed with about 1 μg DNA encoding the VA RNA gene [Thimmappaya et al., Cell, 31:543 (1982)] and dissolved in 500 μl of 1 mM Tris-HCl, 0.1 mM EDTA, 0.227 M CaCl₂. To this mixture is added, dropwise, 500 μl of 50 mM HEPES (pH 7.35), 280 mM NaCl, 1.5 mM NaPO₄, and a precipitate is allowed to form for 10 minutes at 25° C. The precipitate is suspended and added to the 293 cells and allowed to settle for about four hours at 37° C. The culture medium is aspirated off and 2 ml of 20% glycerol in PBS is added for 30 seconds. The 293 cells are then washed with serum free medium, fresh medium is added and the cells are incubated for about 5 days.

Approximately 24 hours after the transfections, the culture medium is removed and replaced with culture medium (alone) or culture medium containing 200 μCi/ml 35S-cysteine and 200 μCi/ml ³⁵S-methionine. After a 12 hour incubation, the conditioned medium is collected, concentrated on a spin filter, and loaded onto a 15% SDS gel. The processed gel may be dried and exposed to film for a selected period of time to reveal the presence of the GDM polypeptides. The cultures containing transfected cells may undergo further incubation (in serum free medium) and the medium is tested in selected bioassays.

In an alternative technique, DNA encoding the GDM polyeptides may be introduced into 293 cells transiently using the dextran sulfate method described by Somparyrac et al., Proc. Natl. Acad. Sci., 12:7575 (1981). 293 cells are grown to maximal density in a spinner flask and 700 μg pRK5-GDM DNA is added. The cells are first concentrated from the spinner flask by centrifugation and washed with PBS. The DNA-dextran precipitate is incubated on the cell pellet for four hours. The cells are treated with 20% glycerol for 90 seconds, washed with tissue culture medium, and re-introduced into the spinner flask containing tissue culture medium, 5 μg/ml bovine insulin and 0. 1 μg/ml bovine transferrin. After about four days, the conditioned media is centrifuged and filtered to remove cells and debris. The sample containing expressed GDM can then be concentrated and purified by any selected method, such as dialysis and/or column chromatography.

In another embodiment, the GDM polypeptide can be expressed in CHO cells. The pRK5-GDM can be transfected into CHO cells using known reagents such as CaPO₄ or DEAE-dextran. As described above, the cell cultures can be incubated, and the medium replaced with culture medium (alone) or medium containing a radiolabel such as ³⁵S-methionine. After determining the presence of the GDM, the culture medium may be replaced with serum free medium. Preferably, the cultures are incubated for about 6 days, and then the conditioned medium is harvested. The medium containing the expressed GDM polypeptide can then be concentrated and purified by any selected method.

Epitope-tagged GDM polypeptide may also be expressed in host CHO cells. The sequence encoding the GDM portion may be subcloned out of the pRK5 vector. The subclone insert can undergo PCR to fuse in frame with a selected epitope tag such as a poly-his tag into a Baculovirus expression vector. This poly-his tagged GDM insert can then be subcloned into a SV40 driven vector containing a selection marker such as DHFR for selection of stable clones. Finally, the CHO cells can be transfected (as described above) with the SV40 driven vector. Labeling may be performed, as described above, to verify expression. The culture medium containing the expressed poly-His tagged GDM can then be concentrated and purified by any selected method, such as by Ni²⁺-chelate affinity chromatography.

GDM polypeptide may also be expressed in CHO and/or COS cells by a transient expression procedure or in CHO cells by another stable expression procedure.

Stable expression in CHO cells is performed using the following procedure. The proteins are expressed as an IgG construct (immunoadhesin), in which the coding sequences for the soluble forms (e.g. extracellular domains) of the respective proteins are fused to an IgG1 constant region sequence containing the hinge, CH2 and CH2 domains and/or is a poly-His tagged form.

Following PCR amplification, the respective DNAs are subcloned in a CHO expression vector using standard techniques as described in Ausubel et al., Current Protocols of Molecular Biology, Unit 3.16, John Wiley and Sons (1997). CHO expression vectors are constructed to have compatible restriction sites 5′ and 3′ of the DNA of interest to allow the convenient shuttling of cDNA's. The vector used expression in CHO cells is as described in Lucas et al., Nucl. Acids Res. 24:9 (1774-1779 (1996), and uses the SV40 early promoter/enhancer to drive expression of the cDNA of interest and dihydrofolate reductase (DHFR). DHFR expression permits selection for stable maintenance of the plasmid following transfection.

Twelve micrograms of the desired plasmid DNA is introduced into approximately 10 million CHO cells using commercially available transfection reagents SUPERFECT® (Quiagen), DOSPER® or FUGENE® (Boehringer Mannheim). The cells are grown as described in Lucas et al., supra. Approximately 3×10⁷ cells are frozen in an ampule for further growth and production as described below.

The ampules containing the plasmid DNA are thawed by placement into water bath and mixed by vortexing. The contents are pipetted into a centrifuge tube containing 10 mLs of media and centrifuged at 1000 rpm for 5 minutes. The supernatant is aspirated and the cells are resuspended in 10 mL of selective media (0.2 μm filtered PS20 with 5% 0.2 μm diafiltered fetal bovine serum). The cells are then aliquoted into a 100 mL spinner containing 90 mL of selective media. After 1-2 days, the cells are transferred into a 250 mL spinner filled with 150 mL selective growth medium and incubated at 37° C. After another 2-3 days, 250 mL, 500 mL and 2000 mL spinners are seeded with 3×10⁵ cells/mL. The cell media is exchanged with fresh media by centrifugation and resuspension in production medium. Although any suitable CHO media may be employed, a production medium described in U.S. Pat. No. 5,122,469, issued Jun. 16, 1992 may actually be used. A 3L production spinner is seeded at 1.2×10⁶ cells/mL. On day 0, the cell number pH ie determined. On day 1, the spinner is sampled and sparging with filtered air is commenced. On day 2, the spinner is sampled, the temperature shifted to 33° C., and 30 mL of 500 g/L glucose and 0.6 mL of 10% antifoam (e.g., 35% polydimethylsiloxane emulsion, Dow Corning 365 Medical Grade Emulsion) taken. Throughout the production, the pH is adjusted as necessary to keep it at around 7.2. After 10 days, or until the viability dropped below 70%, the cell culture is harvested by centrifugation and filtering through a 0.22 μm filter. The filtrate was either stored at 4° C. or immediately loaded onto columns for purification.

For the poly-His tagged constructs, the proteins are purified using a Ni-NTA column (Qiagen). Before purification, imidazole is added to the conditioned media to a concentration of 5 mM. The conditioned media is pumped onto a 6 ml Ni-NTA column equilibrated in 20 mM Hepes, pH 7.4, buffer containing 0.3 M NaCl and 5 mM imidazole at a flow rate of 4-5 ml/min. at 4° C. After loading, the column is washed with additional equilibration buffer and the protein eluted with equilibration buffer containing 0.25 M imidazole. The highly purified protein is subsequently desalted into a storage buffer containing 10 mM Hepes, 0.14 M NaCl and 4% mannitol, pH 6.8, with a 25 ml G25 Superfine (Pharmacia) column and stored at −80° C.

Immunoadhesin (Fc-containing) constructs are purified from the conditioned media as follows. The conditioned medium is pumped onto a 5 ml Protein A column (Pharmacia) which had been equilibrated in 20 mM Na phosphate buffer, pH 6.8. After loading, the column is washed extensively with equilibration buffer before elution with 100 mM citric acid, pH 3.5. The eluted protein is immediately neutralized by collecting 1 ml fractions into tubes containing 275 μL of 1 M Tris buffer, pH 9. The highly purified protein is subsequently desalted into storage buffer as described above for the poly-His tagged proteins. The homogeneity is assessed by SDS polyacrylamide gels and by N-terminal amino acid sequencing by Edman degradation.

### Example 12

Expression of GDM in Yeast

The following method describes recombinant expression of GDM polypeptide in yeast.

First, yeast expression vectors are constructed for intracellular production or secretion of the preceding GDM sequences from the ADH2/GAPDH promoter. DNA encoding such GDM sequences and the promoter is inserted into suitable restriction enzyme sites in the selected plasmid to direct intracellular expression of GDM. For secretion, DNA encoding such GDM sequences can be cloned into the selected plasmid, together with DNA encoding the ADH2/GAPDH promoter, a native GDM signal peptide or other mammalian signal peptide, or, for example, a yeast alpha-factor or invertase secretory signal/leader sequence, and linker sequences (if needed) for expression of GDM.

Yeast cells, such as yeast strain AB110, can then be transformed with the expression plasmids described above and cultured in selected fermentation media. The transformed yeast supernatants can be analyzed by precipitation with 10% trichloroacetic acid and separation by SDS-PAGE, followed by staining of the gels with Coomassie Blue stain.

Recombinant GDM can subsequently be isolated and purified by removing the yeast cells from the fermentation medium by centrifugation and then concentrating the medium using selected cartridge filters. The concentrate containing GDM may further be purified using selected column chromatography resins.

### Example 13

Expression of GDM in Baculovirus-Infected Insect Cells

The following method describes recombinant expression of GDM polypeptide in Baculovirus-infected insect cells.

The sequence coding for the preceding GDM sequence is fused upstream of an epitope tag contained within a baculovirus expression vector. Such epitope tags include poly-his tags and immunoglobulin tags (like Fc regions of IgG). A variety of plasmids may be employed, including plasmids derived from commercially available plasmids such as pVL1393 (Novagen). Briefly, the sequence encoding the preceding GDM sequence or the desired portion of the coding sequence of such, e.g. the sequence encoding an extracellular domain of a transmembrane protein or the sequence encoding the mature protein if the protein is extracellular, is amplified by PCR with primers complementary to the 5′ and 3′ regions. The 5′ primer may incorporate flanking (selected) restriction enzyme sites. The product is then digested with those selected restriction enzymes and subcloned into the expression vector.

Recombinant baculovirus is generated by co-transfecting the above plasmid and BACULOGOLD™ virus DNA (Pharmingen) into Spodoptera frugiperda (“Sf9”) cells (ATCC CRL 1711) using lipofectin (commercially available from GIBCO-BRL). After 4-5 days of incubation at 28° C., the released viruses are harvested and used for further amplifications. Viral infection and protein expression are performed as described by O'Reilley et al., Baculovirus expression vectors: A Laboratory Manual, Oxford: Oxford University Press (1994).

Expressed poly-his tagged GDM polypeptide can then be purified, for example, by Ni²⁺-chelate affinity chromatography as follows. Extracts are prepared from recombinant virus-infected Sf9 cells as described by Rupert et al., Nature, 362:175-179 (1993). Briefly, Sf9 cells are washed, resuspended in sonication buffer (25 mL Hepes, pH 7.9; 12.5 mM MgCl₂; 0.1 mM EDTA; 10% glycerol; 0.1% NP-40; 0.4 M KCl), and sonicated twice for 20 seconds on ice. The sonicates are cleared by centrifugation, and the supernatant is diluted 50-fold in loading buffer (50 mM phosphate, 300 mM NaCl, 10% glycerol, pH 7.8) and filtered through a 0.45 μm filter. A Ni²⁺-NTA agarose column (commercially available from Qiagen) is prepared with a bed volume of 5 mL, washed with 25 mL of water and equilibrated with 25 mL of loading buffer. The filtered cell extract is loaded onto the column at 0.5 mL per minute. The column is washed to baseline A₂₈₀ with loading buffer, at which point fraction collection is started. Next, the column is washed with a secondary wash buffer (50 mM phosphate; 300 mM NaCl, 10% glycerol, pH 6.0), which elutes nonspecifically bound protein. After reaching A₂₈₀ baseline again, the column is developed with a 0 to 500 mM Imidazole gradient in the secondary wash buffer. One mL fractions are collected and analyzed by SDS-PAGE and silver staining or Western blot with Ni²⁺-NTA-conjugated to alkaline phosphatase (Qiagen). Fractions containing the eluted His₁₀-tagged GDM polypeptide are pooled and dialyzed against loading buffer.

Alternatively, purification of the IgG tagged (or Fc tagged) GDM polypeptide can be performed using known chromatography techniques, including for instance, Protein A or protein G column chromatography.

### Example 14

Purification of GDM Polypeptide Using Specific Antibodies

Native or recombinant GDM polypeptides may be purified by a variety of standard techniques in the art of protein purification. For example, pro-, mature or pre-polypeptide variants of the preceding GDM sequences are purified by immunoaffinity chromatography using antibodies specific for such sequences. In general, an immunoaffinity column is constructed by covalently coupling the anti-GDM antibody to an activated chromatographic resin.

Polyclonal immunoglobulins are prepared from immune sera either by precipitation with ammonium sulfate or by purification on immobilized Protein A (Pharmacia LKB Biotechnology, Piscataway, N.J.). Likewise, monoclonal antibodies are prepared from mouse ascites fluid by ammonium sulfate precipitation or chromatography on immobilized Protein A. Partially purified immunoglobulin is covalently attached to a chromatographic resin such as CnBr-activated SEPHAROSE™ (Pharmacia LKB Biotechnology). The antibody is coupled to the resin, the resin is blocked, and the derivative resin is washed according to the manufacturer's instructions.

Such an immunoaffinity column is utilized in the purification of the preceding GDM sequences by preparing a fraction from cells containing such sequences in a soluble form. This preparation is derived by solubilization of the whole cell or of a subcellular fraction obtained via differential centrifugation by the addition of detergent or by other methods well known in the art. Alternatively, soluble GDM polypeptide containing a signal sequence may be secreted in useful quantity into the medium in which the cells are grown.

A soluble GDM polypeptide-containing preparation is passed over the immunoaffinity column, and the column is washed under conditions that allow the preferential absorbance of such sequences (e.g., high ionic strength buffers in the presence of detergent). Then, the column is eluted under conditions that disrupt the binding between the antibody/substrate (e.g., a low pH buffer such as approximately pH 2-3, or a high concentration of a chaotrope such as urea or thiocyanate ion), and GDM polypeptide, respectively, is collected.

1. A method for treating a glioma tumor comprising:
(a) measuring the expression of a set of GDM in a sample of the tumor;
(b) determining the subclassification, PN, Prolif or Mes of the tumor; and
(c) contacting with at least an effective amount of therapeutic based on the subclassification;
wherein (I) tumors exhibiting a Prolif subclassification are treated with a combined therapy comprising contacting with effective amounts of (a) an Akt antagonist and/or a Prolif-antagonist and/or anti-mitotic agent, and (b) a neural differentiation agent; (II) tumors exhibiting a Mes subclassification are treated with a combined therapy comprising contacting with effective amounts of (a) an Akt and/or Mes-antagonist and/or anti-angiogenic agent, and (b) a neural differentiation agent; and (III) tumors exhibiting a PN subclassification are treated with a combined therapy comprising contacting with effective amounts of: (1) a PN-antagonist; and/or (2) a neural differentiation agent; optionally in combination with one or more of the following: (3) an Akt antagonist; (4) an anti-mitotic agent and (5) an anti-angiogenic agent.

2. The method of claim 1, wherein the subclassification is carried out by comparing the tumor to a set of glioma samples using hierarchical clustering.

3. The method of claim 1, wherein the subclassification is carried out by comparing the tumor to a set of samples using k means clustering.

4. The method of claim 1, wherein the subclassification is carried out by comparing the tumor to a set of samples using a voting scheme.

5. The method of claim 1, wherein the subclassification is carried out by comparing the similarity of expression of a set of GDM markers between the tumor and a set of previously classified glioma samples.

6. The method of claim 1, wherein the PN antagonist is selected from the group consisting of: the PN markers indicated in Table A, with the exception of DLL3, Nog, Olig1, Olig2, THR and ASCL1.

7. The method of claim 1, wherein the Prolif antagonist is selected from the group consisting of: antagonists of any of the Prolif markers indicated in Table A.

8. The method of claim 1, wherein the Mes antagonist is selected from the group consisting of: antagonists of any of the Mes markers indicated in Table A.

9. The method of claim 1, wherein the Akt antagonist is selected from the group consisting of: antagonists of akt1, akt2, akt3, antagonists of regulatory or catalytic domain of PIK3, PD1, FRAP, RPS6KB1, SGK, EGFR, IGFR, and activators, stimulators or restoratives of PTEN, INPP5D or INPPL1.

10. The method of claim 1, wheren the anti-mitotic agent is selected from the group consisting of: temozolamide, BCNU, CCNU, lomustine, gliadel, etoposide, carmustine, ironotecan, topotecan, procarbazine, cisplatin, carboplatin, cyclophosphamide, vincristine, doxorubicin, dactinomycin, bleomycin, plicamycin, methotrexate, cytarabine, paclitaxel, auristatins, maytansinoids.

11. The method of claim 1, wherein the anti-angiogenic agent is selected from the group consisting of: VEGF antagonists, anti-VEGF antibody, VEGFR1 and VEGFR2 antagonists.

12. The method of claim 1, wherein the neuronal differentiation agent is selected from the group consisting of: MAP2, beta-tubulin, GAD65 and GAP43. Example neural differentiation agents include, but are not limited to: retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-4, jagged 1 antagonist, jagged 2 antagonist; numb agonist or numb-like agonist.

13. A method of prognosing and/or diagnosing glioma comprising:
(a) measuring the expression of a set of GDM;
(b) determining the subclassification, PN, Prolif or Mes of the tumor, and
(c) prognosing and/or diagnosing disease outcome;
wherein a subclassification of Prolif or Mes is indicative of poorer prognosis or shortened survival time and a subclassification of PN is indicative of a better prognosis or lengthened survival time.

14. The method of claim 7, wherein the subclassification is carried out by comparing the tumor to a set of samples using hierarchical clustering.

15. The method of claim 7, wherein the subclassification is carried out by comparing the tumor to a set of samples using k means clustering.

16. The method of claim 7, wherein the subclassification is carried out by comparing the tumor to set of samples using a voting scheme.

17. The method of claim 7, wherein the subclassification is carried out by comparing similarity of expression of a set of GDM markers between the tumor and a set of previously classified glioma samples.

18. A method of prognosing and/or diagnosing glioma comprising:
(a) measuring the expression of PTEN and DLL3 tumor markers in a tumor sample, and
(b) prognosing and/or diagnosing based on the expression of said tumor markers,
wherein a higher expression of both PTEN and DLL3 indicates a better prognosis or lengthened survival time, and a lower expression level of PTEN regardless of DLL3 expression level indicates a worse prognosis or shortened survival time.

19. A method of monitoring or diagnosing glioma comprising comparing the expression signature of a set of glioma determinative markers (“GDM”) in at least two samples from a patient, comprising the steps of:
(a) measuring the expression of GDM in a first tumor sample at a first point in time;
(b) measuring the expression of GDM in a second tumor sample at a second later point in time; and
(c) determining the subclassification, PN, Prolif or Mes, in the first and second samples;
wherein a transition from the PN or Prolif to Mes subclassification from the first to the second tumor sample is indicative of increased severity or progression of said tumor.

20. A method of inhibiting the size or growth of a glioma tumor comprising:
(a) measuring the expression of a set of GDM in a sample of the tumor;
(b) determining the subclassification, PN, Prolif or Mes of the tumor; and
(c) contacting with at least an effective amount of therapeutic based on the subclassification;
wherein (I) tumors exhibiting a Prolif subclassification are treated with a combined therapy comprising contacting with effective amounts of (a) an Akt antagonist and/or a Prolif-antagonist and/or anti-mitotic agent, and (b) a neural differentiation agent; (II) tumors exhibiting a Mes subclassification are treated with a combined therapy comprising contacting with effective amounts of (a) an Akt and/or Mes-antagonist and/or anti-angiogenic agent, and (b) a neural differentiation agent; and (III) tumors exhibiting a PN subclassification are treated with a combined therapy comprising contacting with effective amounts of: (1) a PN-antagonist; and/or (2) a neural differentiation agent; optionally in combination with one or more of the following: (3) an Akt antagonist; (4) an anti-mitotic agent and (5) an anti-angiogenic agent; and wherein the results is the reduced size or growth of the tumor.

21. The method of claim 20, wherein the subclassification is carried out by comparing the tumor to a set of glioma samples using hierarchical clustering.

22. The method of claim 20, wherein the subclassification is carried out by comparing the tumor to a set of glioma samples using k means clustering.

23. The method of claim 20, wherein the subclassification is carried out by comparing the tumor to a set of glioma samples using a voting scheme.

24. The method of claim 20, wherein the subclassification is carried out by comparing the similarity of expression of a set of GDM markers between the tumor and a set of previously classified glioma samples.

25. The method of claim 20, wherein the PN antagonist is selected from the group consisting of an antagonist of any of: the PN markers indicated in Table A, with the exception of DLL3, Nog, Olig1, Olig2, THR and ASCL1.

26. The method of claim 20, wherein the Prolif antagonist is selected from the group consisting of: antagonists of any of the Prolif markers indicated in Table A.

27. The method of claim 20, wherein the Mes antagonist is selected from the group consisting of: antagonists of any of the Mes markers indicated in Table A.

28. The method of claim 20, wherein the Akt antagonist is selected from the group consisting of: antagonists of akt1, akt2, akt3, antagonists of regulatory or catalytic domain of PIK3, PD1, FRAP, RPS6KB1, SGK, EGFR, IGFR, and activators, stimulators or restoratives of PTEN, INPP5D or INPPL1.

29. The method of claim 20, wherein the anti-angiogenic agent is selected from the group consisting of: VEGF antagonists, anti-VEGF antibody, VEGFR1 and VEGFR2 antagonists.

30. The method of claim 20, wherein the anti-mitotic agent is selected from the group consisting of: temozolamide, BCNU, CCNU, lomustine, gliadel, etoposide, carmustine, ironotecan, topotecan, procarbazine, cisplatin, carboplatin, cyclophosphamide, vincristine, doxorubicin, dactinomycin, bleomycin, plicamycin, methotrexate, cytarabine, paclitaxel, auristatins, maytansinoids.

31. The method of claim 20, wherein the neural differentiation agent is selected from the group consisting of: MAP2, beta-tubulin, GAD65 and GAP43. Example neural differentiation agents include, but are not limited to: retinoic acid, valproic acid and derivatives thereof (e.g., esters, salts, retinoids, retinates, valproates, etc.); thyroid hormone or other agonists of thyroid hormone receptor; noggin; BDNF, NT 4/5 or other agonists of the NTRK2 receptor; agents which increase expression of the transcription factors ASCL1, OLIG1; d113 agonists, Notch 1, 2, 3 or 4 antagonists, gamma secretase inhibitors, including small molecule inhibitors of nicastrin, Aph1A, Aph1B, Psen1, Psen2 and PSENEN, delta like ligand (D11)-1 antagonist, delta like ligand (D11)-4, jagged 1 antagonist, jagged 2 antagonist; numb agonist or numb-like agonist.

32. The method of claim 20, wherein the contact with the antagonist and/or agent results in the death of the tumor cell.

33. The method of claim 20, wherein the PN-, Prolif- or Mes-antagonist is (1) an anti-PN-, anti-Prolif- or anti-Mes antibody, (2) an anti-PN-, anti-Prolif- or anti-Mes antigen-binding fragment, (3) a PN-, Prolif- or Mes- binding oligopeptide, (4) a PN-, Prolif- or Mes- small molecule antagonist or (5) PN-, Prolif- or Mes- antisense oligonucleotide.

34. The method of claim 20, wherein the PN-, Prolif- or Mes- antagonist is selected from the group consisting of: (1) an anti-PN-, anti-Prolif- or anti-Mes antibody, and (2) an anti-PN-, anti-Prolif- or anti-Mes antigen-binding fragment.

35. The method of claim 20, wherein the antagonist antibody is selected from the group consisting of: monoclonal antibody, chimeric antibody, humanized antibody and single-chain antibody.

36. The method of claim 20 wherein the antibody or antigen-binding fragment is conjugated to a growth inhibitory agent or cytotoxic agent.

37. The method of claim 20, wherein the growth inhibitory agent or cytotoxic agent is selected from the group consisting of: maytansinoid, calicheamicin, antibiotic, radioactive isotope and nucleolytic enzyme.

38. A method for therapeutically treating a mammal having a glioma tumor, wherein the method comprises:
(a) measuring the expression of a set of GDM in a sample of the tumor;
(b) determining the subclassification, PN, Prolif or Mes of the tumor; and
(c) contacting with at least an effective amount of therapeutic based on the subclassification;
wherein (I) tumors exhibiting a Prolif subclassification are treated with a combined therapy comprising administering to the mammal therapeutically effective amounts of (a) an Akt antagonist and/or a Prolif-antagonist and/or anti-mitotic agent, and (b) a neural differentiation agent; (II) tumors exhibiting a Mes subclassification are treated with a combined therapy comprising contacting with effective amounts of (a) an Akt and/or Mes-antagonist and/or anti-angiogenic agent, and (b) a neural differentiation agent; and (III) tumors exhibiting a PN subclassification are treated with a combined therapy comprising contacting with effective amounts of: (1) a PN-antagonist; and/or (2) a neural differentiation agent; optionally in combination with one or more of the following: (3) an Akt antagonist; (4) an anti-mitotic agent and (5) an anti-angiogenic agent; and wherein the result is the reduced size or growth of the tumor.

39. The method of claim 38, wherein the administration of the antagonist or agent results in the death of the glioma tumor.

40. The method of claim 38, wherein the antagonist or agent is an antibody, an anti-antigen-binding antibody fragment, an oligopeptide, a small molecule antagonist or antisense oligonucleotide.

41. The method of claim 40, wherein the antagonist antibody is selected from the group consisting of: monoclonal antibody, chimeric antibody, humanized antibody and single-chain antibody.

42. The method of claim 40 wherein the antibody or antigen-binding fragment is conjugated to a growth inhibitory agent or cytotoxic agent.

43. The method of claim 42, wherein the growth inhibitory agent or cytotoxic agent is selected from the group consisting of: maytansinoid, calicheamicin, antibiotic, radioactive isotope and nucleolytic enzyme.

44. A method for determining the expression level of PN-, Prolif- or Mes- Glioma Determinative Markers (“GDM”) in a sample, wherein the method comprises exposing the sample to PN-, Prolif- or Mes- binding agents, and determining the amount of binding of each in the sample, wherein the binding amount is indicative of the expression level of the respective PN-, Prolif- or Mes- GDM in the sample.

45. The method of claim 44, wherein the PN-, Prolif- or Mes- binding agent is selected from the group consisting of: anti- PN-, Prolif- or Mes- antibody; PN-, Prolif- or Mes- binding antibody fragment; PN-, Prolif- or Mes- oligopeptide, PN-, Prolif- or Mes-small molecule antagonist and PN-, Prolif- or Mes- antisense oligonucleotide.

46. The method of claim 44, wherein the anti-PN-, anti-Prolif- or anti-Mes- antibody is selected from the group consisting of: monoclonal antibody, antigen-binding antibody fragment, chimeric antibody, humanized antibody and single-chain antibody.

47. The method of claim 45 wherein the PN-, Prolif- or Mes- binding agent is detectably labeled.

48. A method of prognosing the survival time in a mammal having a glioma tumor, wherein the method comprises:
a) removing a test sample of the tumor,
b) measuring the level of PTEN and DLL3 expression in the test sample and in a set of not less than thirty (30) high grade gliomas for which patient survival times are known,
wherein a higher level of expression of both PTEN and DLL3 in the test sample is indicative of a statistically elevated chance of survival time greater than the median of the reference sample population and a lower level of expression of either PTEN or DLL3 in the test sample is indicative of a statistically elevated chance of survival time less than the median of the reference sample population.

49. A method of diagnosing the severity of a glioma tumor in a mammal, wherein the method comprises:
(a) contacting a test sample comprising tissue obtained from the mammal with:
(i) a first reagent which is an antibody, oligopeptide or small organic molecule that binds to a PTEN polypeptide, and
(ii) a second reagent which is an antibody, oligopeptide or small organic molecule that binds to a DLL3 polyeptide;
(b) measuring the amount of complex formation between the first and second reagents with the PTEN and DLL3 polypeptides in the test sample, respectively,
wherein the formation of a high amount of both PTEN and DLL3 complex formation is indicative of a mild tumor and the formation of a low amount of either PTEN or DLL3 complex formation is indicative of a severe tumor.

50. The method of claim 49, wherein the first and/or second reagant are/is detectably labeled.

51. The method of claim 50, wherein the first and/or second reagent are/is attached to a solid support.

52. A use of: (a) a PN-, Prolif- or Mes- GDM polypeptide, or (b) a nucleic acid sequence encoding (a), in the preparation of a medicament useful for (i) the therapeutic treatment or (ii) diagnostic detection of a glioma tumor.

53. The use of claim 52, wherein the GDM polypeptide is an antibody, a GDM binding antibody fragment, a GDM binding oligopeptide, a GDM small molecule antagonist, or a GDM antisense oligonucleotide.

54. The use of either claim 52 or 53, wherein the antibody is a monoclonal antibody, antigen-binding antibody fragment, chimeric antibody, humanized antibody or single-chain antibody.

55. A method for therapeutically treating a mammal having a glioma tumor, wherein the method comprises contacting with effective amounts of a neural differentiation agent; in combination with one or more of the following: (1) an Akt antagonist; (2) an anti-mitotic agent and (3) an anti-angiogenic agent; and wherein the result is the reduced size or growth of the tumor.
